(19) United States
(12) Patent Application Publication
(10) Pub. No.: US 2003/0219739 A1
(43) Pub. Date: Nov. 27, 2003

(54) Novel nucleic acid and polypeptide molecules

(76) Inventors: David J. Glass, Cortlandt Manor, NY (US); Sue C. Bodine, West Harrison, NY (US)

Correspondence Address: Laura J. Fischer, Regeneron Pharmaceuticals, Inc., 777 Old Saw Mill River Road, Tarrytown, NY, 10591, US

(21) Appl. No.: 10/060,634

(22) Filed: Jan. 30, 2002

Related U.S. Application Data
(60) Provisional application No. 60/264,926, filed on Jan. 30, 2001.
(60) Provisional application No. 60/311,697, filed on Aug. 10, 2001.
(60) Provisional application No. 60/338,742, filed on Oct. 22, 2001.

(51) Int. Cl. C12Q 1/68; A01K 67/00; C07H 21/04; C12P 21/02; C12N 5/06; C07K 14/47; A01K 67/027

(52) U.S. Cl. 435/6; 435/69.1; 435/320.1; 435/325; 530/350; 536/23.2; 800/9

(57) ABSTRACT

The present invention provides for nucleic acid sequences that encode novel mammalian intracellular signaling polypeptides, designated MURF1, MURF3, or MA-61. The invention also provides assay systems that may be used to detect and/or measure agents that bind the MURF1 or MAFBXgene product. The present invention also provides for diagnostic and therapeutic methods based on the interaction between MURF1 or MAFBXand agents that initiate signal transduction or inhibition of ubiqutination through binding to MURF1 or MA-61, inhibiting the mRNA expression of MURF1, MURF3, or MA-61, or inhibiting the MURF, MURF3, or MAFBXpathway.

Throughout this application, various publications are referenced. The disclosures of these publications in their entireties are hereby incorporated by reference into this application. This application claims priority to provisional applications U.S. Application Nos. 60/264,926 filed Jan. 30, 2001, 60/311,697 filed Aug. 10, 2001, and 60/338,742 filed Oct. 22, 2001.

### INTRODUCTION

This invention relates to novel human nucleotide sequences. Two of these, herein designated MURF1 and MA-61, encode novel substrate-targeting subunits of ubiquitin ligases and are modulated by conditions or agents that either induce, prevent or reverse muscle atrophy. An additional sequence that is highly homologous to MuRF-1 encodes a molecule herein designated MuRF-3 whose substrate is Syncoilin. Induction of atrophy causes an increase in mRNA expression of these genes; reversal or prevention of atrophy decreases or blocks expression of these genes. The MURF1 and MAFBXcDNA sequences, and additional experiments described herein, demonstrate that the MURF1 and MAFBXprotein molecules are involved in ubiquitination, a specific pathway of initiating protein breakdown in the cell. The invention encompasses the nucleic acid molecules which encode MURF1, MURF-3 and/or MA-61, transgenic mice, knock-out mice, host cell expression systems and proteins encoded by the nucleotides of the present invention. The invention further relates to the use of these nucleic acids in screening assays to identify potential therapeutic agents which affect these genes themselves and the proteins they encode, ubiquitination, muscle atrophy and associated diseases, disorders and conditions. In addition, the invention further encompasses therapeutic protocols and pharmaceutical compositions designed to target the ubiquitin pathway and the substrates thereof for the treatment of associated diseases. The molecules disclosed herein function to modulate muscle atrophy or induce muscle hypertrophy.

### BACKGROUND OF THE INVENTION

A decrease in muscle mass, or atrophy, is associated with various physiological and pathological states. For example, muscle atrophy can result from denervation due to nerve trauma; degenerative, metabolic or inflammatory neuropathy, e.g. Guillian-Barré syndrome; peripheral neuropathy; or nerve damage caused by environmental toxins or drugs. Muscle atrophy may also result from denervation due to a motor neuropathy including, for example, adult motor neuron disease, such as Amyotrophic Lateral Sclerosis (ALS or Lou Gehrig's disease); infantile and juvenile spinal muscular atrophies; and autoimmune motor neuropathy with multifocal conductor block. Muscle atrophy may also result from chronic disease resulting from, for example, paralysis due to stroke or spinal cord injury; skeletal immobilization due to trauma, such as, for example, fracture, sprain or dislocation; or prolonged bed rest (R. T. Jagoe, A. L. Goldberg, Curr. Opin. Clin. Nutr. Metab. Care 4,183 (2001). Metabolic stress or nutritional insufficiency, which may also result in muscle atrophy, include inter alia the cachexia of cancer, AIDS, and other chronic illnesses, fasting or rhabdomyolysis, and endocrine disorders such as disorders of the thyroid gland and diabetes. Muscle atrophy may also be due to a muscular dystrophy syndrome such as Duchenne, Becker, myotonic, fascioscapulohumeral, Emery-Dreifuss, oculopharyngeal, scapulohumeral, limb girdle, and congenital types, as well as the dystrophy known as Hereditary Distal Myopathy. Muscle atrophy may also be due to a congenital myopathy, such as benign congenital hypotonia, central core disease, nemalene myopathy, and myotubular (centronuclear) myopathy. Muscle atrophy also occurs during the aging process.

Muscle atrophy in various pathological states is associated with enhanced proteolysis and decreased synthesis of muscle proteins. Muscle cells contain lysosomal proteases and cytosolic proteases. The cytosolic proteases include Ca²⁺-activated neutral proteases (calpains) and an ATP-dependent ubiquitin-proteasome proteolytic system. The lysosomal and cytosolic systems are capable of degrading muscle proteins in vitro, but less is known about their roles in the proteolysis of muscle proteins in vivo. Some studies have reported that proteosome inhibitors reduce proteolysis in atrophying rat skeletal muscle (e.g. Tawa et al. (1997) J. Clin. Invest100:197), leading to suggestions that the ubiquitin-proteasome pathway has a role in the enhanced proteolysis. However, the precise mechanisms of proteolysis in atrophying muscle remain poorly characterized. A better understanding of proteolysis would allow the design of strategies and agents for the prevention and treatment of atrophy.

Protein degradation is a common mechanism used by cells to control protein abundance. However, rather than simply degrading all proteins, ubiquitination seems to be very specific in terms of protein target selection. The formation of such ubiquitin-protein conjugates involves a protein complex consisting of three components: a ubiquitin activating enzyme (E1), a ubiquitin conjugating enzyme (E2), and a substrate specificity determining component (E3) (Skowyra, et al, 1997, Cell 91:209-219). There are several distinct molecular strategies that regulate which protein targets become ubiquitinated. A recently discovered mechanism is referred to as the SCF E3 ubiquitin ligase complex (see FIG. 1 for a schematic representation of the complex). The SCF protein complex comprises several distinct protein subunits, including a protein which has a domain referred to as an “F-box.” In the presence of a phosphorylated substrate, the SCF complex binds to the substrate, and ubiquitinates it, using an E2 ubiquitin transferase which is also part of the SCF complex (Patton, et al, 1998, Genes & Development 12:692-705). The result is the specific proteolytic degradation of the substrate. F-box proteins comprise a large family that can be divided into three subfamilies: 1) Fbws, which are characterized by multiple Trp-Asp repeats (WD-40 repeats); 2) Fbls, which are characterized by leucine-rich repeat; and 3) Fbxs, which lack known protein interaction domains (see Winston, et al, 1999, Current Biology 9:1180-1182 for a discussion of the currently known mammalian F-box protein family members). F-box proteins usually contain an additional substrate-binding domain that interacts with specific protein substrates and a 42-48 amino acid motif termed the F-box (Winston, 1999). See FIG. 2 for a comparison of hMAFBXwith other F-box-containing proteins.

Another mechanism for ligation of ubiquitin to specific substrates involves proteins which contain a “ring-domain.” Ring-domain proteins can either act as independent monomeric ubiquitin ligases, or they can function as part of an SCF complex. As with F-box proteins, ring-domain proteins usually contain a second domain which binds specific substrates. The ring-domain recruits the ubiquitin ligase. The net result is the ubiquitination of the substrate, resulting in proteolysis.

Another protein complex involved in the maintenance of normal muscle tissue is the dystrophin protein complex, which is thought to play an integral role in the link between the extracellular matrix of the muscle cell and the actin cytoskeleton. A key component of the dystrophin protein complex is a-dystrobrevin, a dystrophin-associated protein whose absence results in neuromuscular junction defects and muscular dystrophy. Recently a novel a-dystrobrevin-binding partner called Syncoilin has been identified. (Newey, et al, JBC Papers in Press, Oct. 25, 2000). Syncoilin is a member of the intermediate filament family. It is highly expressed in skeletal and cardiac muscle, and is concentrated at the neuromuscular junction.

In accordance with the present invention, novel protein molecules termed MURF1 (formerly called MUSCLE ATROPHY-16 or MA-16), MURF3, and MUSCLE ATROPHY-61 (MA-61), have been discovered. MAFBXis a novel F-box protein (see FIG. 3 for a schematic representation) that is specifically expressed in skeletal muscle and heart, and, to a lesser degree, certain areas of the brain. The level of expression of MAFBXmRNA increases significantly during skeletal muscle atrophy. MURF1 is a novel ring domain protein (see FIG. 4 for a schematic representation) that is specifically expressed in skeletal muscle and heart. The level of expression of MURF1 mRNA increases significantly during skeletal muscle atrophy. Therefore, it has been discovered in accordance with the present invention that mRNA expression of MURF1 or MAFBXprovide unique markers for muscle atrophy. MURF3 is a novel ring domain protein, whose substrate is Syncoilin which is involved in the dystrophin protein complex. Because this complex is involved in the maintenance of normal muscle tissue, MURF-3 may also be useful in the prevention of atrophy, as well as other diseases and complications of the musculature. The present discovery allows for the identification of agents for the treatment and prevention of atrophy as well as identification of a pathway useful for targeting agents for the treatment and prevention of atrophy. The present invention provides general insight into normal muscle functioning, particularly with regards to the SCF protein complex and the dystrophin complex.

### SUMMARY OF THE INVENTION

The present invention provides for the protein and nucleic acid sequences of novel mammalian intracellular signaling molecules, termed MURF1, MURF 3, and MUSCLE ATROPHY-61 (MA-61), and the therapeutic protocols and compositions utilizing such molecules in the treatment of muscle atrophy and other related conditions. The present invention relates to screening assays to identify substrates of these molecules and to the identification of agents which modulate or target these molecules, ubiquitination or the ubiquitin pathway, or the dystrophin complex. These screening assays may be used to identify potential therapeutic agents for the treatment of muscle atrophy and related disorders.

The present invention provides for the protein or polypeptide that comprises the F-box motif of MAFBXor the ring domain of MURF1 and MURF3 and the nucleic acids which encode such motifs and/or domains.

The invention also describes a co-association between MURF3 nucleic acids and the Syncoilin gene. This interaction provides insight into the functioning of normal muscle cells and in particular the relationship between the dystrophin protein complex, the intermediate filament superfamily, and the ubiquitination protein complex.

The invention additionally describes a novel protein-protein interaction domain of MA-61. This domain was determined by comparing the MAFBXprotein to a previously discovered F-box-containing protein, Fbx25. These two proteins contain an area of homology distinct from the F-box domain. Applicant calls this domain the Fbx25 homology domain. See FIGS. 5A-5B for the comparison of MAFBXwith Fbx25.

The invention further provides for vectors comprising an isolated nucleic acid molecule of MURF1, MURF3, or MAFBXor the F-box motif of MAFBXor the ring domain of MURF1 or MURF3, which can be used to express MURF1, MURF3 or MAFBXpeptides, or the F-box motif of MA-61, or the ring domain of MURF1 or MURF3 nucleic acids, or MURF1, MURF3, or MAFBXproteins in bacteria, yeast, insect or mammalian cells.

Thus the present invention encompasses the following nucleic acid sequences, host cells expressing such nucleic acid sequences and the expression products of such nucleotide sequences: (a) nucleotide sequences that encode MURF1, MURF3, or MA-61, including both the human and rat homologues, and their gene products; (b) nucleotide sequences that encode the portions of the novel substrate targeting subunits of the MURF1, MURF3, and MAFBXmolecules, including the F-box motif of MA-61, the ring domain of MURF1 or MURF3, the portion of the MURF3 molecule that co-associates with the Syncoilin gene, and the Fbx25 homology domain of MA-61; (c) nucleotide sequences that encode mutants of the novel molecules MURF1, MURF3, and MAFBXin which all or part of the domain is deleted or altered, and the polypeptide products specified by such nucleotide sequences; (d) nucleotide sequence domains that encode fusion proteins containing the novel ubiquitin pathway molecules or one of the domains fused to another polypeptide, and those encoding novel dystrophin complex proteins or one of those domains fused to another polypeptide, (e) nucleotide sequences that hybridize with any of the above enumerated nucleotide sequences under stringent conditions, (stringent conditions may include, for example, hybridizing in a buffer comprising 30% formamide in 5×SSPE (0.18 M NaCl, 0.01 M NaPO₄, pH 7.7, 0.001 M EDTA) buffer at a temperature of 42° C. and remaining bound when subject to washing at 42° C. with 0.2×SSPE; preferably hybridizing in a buffer comprising 50% formamide in 5×SSPE buffer at a temperature of 42° C. and remaining bound when subject to washing at 42° C. with 0.2×SSPE buffer at 42° C.; or preferably hybridizing in a buffer comprising 20% SDS, 10% BSA, 1M NaPO₄, 0.5M EDTA, pH 8 at a temperature of 60° C. and remaining bound when subject to washing at 65° C. with 2×SSC, 0.1% SDS); and (f) nucleotide sequences that are 65% homologous to the above enumerated nucleotide sequences within block of sequence at least 100 base pair in length.

The present invention further provides for use of the MURF1, MURF3, or MAFBXnucleic acids or proteins, the F-box motif of MA-61, the ring domain of MURF1 or MURF3, the portion of the MURF3 molecule that co-associates with the Syncoilin gene, and the Fbx25 homology domain of MA-61, in screening for drugs or agents that interact with or modulate the ubiquitin pathway, the activity or expression of MURF1, MURF3, or MAFBXnucleic acids or proteins, muscle atrophy, and/or the dystrophin complex. Therefore the present invention provides for the use of MURF1, MURF3, and MAFBXnucleic acids or proteins and/or particular domains thereof to follow or modulate interactions of particular drugs, agents, or molecules in the cell, particularly the muscle cell, but also certain neuronal cells, since MAFBXexpression is also detected in regions of the brain. In particular embodiments, the F-box motif of MAFBXor the ring domain of MURF1 or MURF 3 is utilized to screen molecules or agents for interaction with or modulation of the activity or expression of the MURF1, MURF3, or MAFBXmolecules. In other embodiments, MURF1, MURF3, and MAFBXnucleic acids or proteins are used as markers during assay experiments to find drugs which block or prevent muscle atrophy.

The present invention also provides for the use of MURF1, MURF3, or MAFBXnucleic acids or proteins to decrease ubiquitination and/or muscle atrophy by modulating MURF1, MURF3, or MAFBXprotein or peptide expression or activity, or by effecting MURF1, MURF3, or MAFBXprotein interactions in the cell so as to inhibit ubiquitination.

The invention further encompasses all agonists and antagonists of the novel MURF1, MURF3, and MAFBXmolecules and their subunits, including small molecules, large molecules, mutants that compete with the native MURF1, MURF3, and MAFBXbinding proteins, and antibodies, as well as nucleotide sequences that can be used to inhibit MURF1, MURF3, and MAFBXprotein and peptide expression, including antisense and ribozyme molecules and gene regulatory or replacement constructs, or to enhance MURF1, MURF3, and MAFBXprotein or peptide expression, including expression constructs that place the MURF1, MURF3, or MAFBXgene under the control of a strong promoter sequence, and transgenic animals that express a MURF1, MURF3, or MAFBXtransgene or knock-out animals that do not express the MURF1, MURF3, or MAFBXmolecule.

The invention also provides for (a) nucleic acid probe(s) capable of hybridizing with a sequence included within the sequences of human (h)MURF1, rodent (r)MURF1, (h) MURF 3, (r)MURF 3, (h)MA-61, or (r)MAFBXDNA, useful for the detection of MURF1, MURF3, or MAFBXmRNA—expressing tissue in humans and rodents.

The invention further encompasses screening methods to identify derivatives and analogues of the binding subunits of MURF1, MURF3, and MAFBXwhich modulate the activity of the molecules as potential therapeutics for the prevention of muscle atrophy and related diseases and disorders. The invention provides for methods of screening for proteins that interact with the MURF1, MURF3, and MA-61, or derivatives, fragments, or domains thereof, such as the F-box motif of MA-61, the ring domain of MURF1 and MURF3, the portion of the MURF3 molecule that co-associates with the Syncoilin gene, and the Fbx25 homology domain of MA-61. In accordance with the invention, the screening methods may utilize known assays to identify protein-protein interactions including phage display assays, immunoprecipitation with an antibody that binds to the protein followed by size fractionation analysis, Western analysis, gel electrophoresis, the yeast-two hybrid assay system or variations thereof.

The invention further provides for antibodies, including monoclonal and polyclonal antibodies, directed against MURF1 protein, MURF3 protein, or MAFBXprotein, or the F-box motif of MAFBXprotein, or the ring domain of MURF1 or MURF 3 protein, or a fragment or derivative thereof.

The present invention also has diagnostic and therapeutic utilities. Such methods may utilize the gene sequences and/or the gene product sequences for diagnostic or genetic testing. In particular embodiments of the invention, methods of detecting the expression of MURF1, MURF3, or MAFBXmRNA or methods of detecting MURF1, MURF3, or MAFBXproteins described herein may be used in the diagnosis of skeletal muscle atrophy in association with a variety of illnesses, syndromes or disorders, cardiac or skeletal, including those affecting the neuromuscular junction. Mutations in molecules modulating or targeting the ubiquitin pathway may be detected and a subject may be evaluated for risk of developing a muscle atrophy related disease or disorder.

In other embodiments, manipulation of MURF1, MURF3, or MAFBXmRNA expression, or other agents which interact with or modulate the activity or expression of these genes or gene-products, may be employed in the treatment of illnesses, syndromes or disorders associated with muscle atrophy and dystrophy, for example, skeletal or cardiac muscle disorders. Further, the measurement or analysis of MURF1, MURF3, or MAFBXnucleic acids or proteins levels or activity could be used in other embodiments to determine whether pharmacological agents perturb the atrophy process; an increase in expression would correlate to an increase in protein breakdown, whereas a decrease or blockage of expression would correlate to effective decrease or blockade of muscle protein breakdown. In further embodiments, the F-box motif of MAFBXor the ring domain of MURF1 or MURF3 may be manipulated for the treatment of illnesses, syndromes or disorders associated with muscle atrophy and dystrophy, for example, skeletal or cardiac muscle disorders.

The invention further comprises a method of inhibiting atrophy in muscle cells comprising contacting the cells with an inhibitor of MURF1, MURF3, or MAFBXproteins or nucleic acids, an inhibitor of a MURF1, MURF3, or MAFBXpathway, or an inhibitor of ubiquitination. The invention further comprises a method of inhibiting atrophy in muscle cells comprising contacting the cells with an inhibitor of muscle atrophy, resulting in a decrease in expression of MURF1, MURF3, or MAFBXnucleic acids or proteins or activity of MURF1, MURF3, or MAFBXpeptides or proteins. In this embodiment, expression of MURF1, MURF3, or MAFBXnucleic acids or proteins or activity of MURF1, MURF3, or MAFBXpeptides or proteins would be used as a marker to verify the efficacy of the test compound in inhibiting muscle atrophy or the diseases associated therewith.

The invention further provides for a method for screening for agents useful in the treatment of a disease or disorder associated with muscle atrophy comprising contacting a cell expressing MURF1, MURF3 or MAFBXhaving the amino acid sequence of FIGS. 7, 9, 11, 13, 17, 19, and 22, respectively, or a fragment thereof, and its substrate, with a compound and detecting a change in the activity of either MURF1, MURF3, or MAFBXgene products. Such change in activity may be manifest by a change in the interaction of MURF1, MURF3, or MAFBXgene products with one or more proteins, such as one of their substrates or a component of the ubiquitin pathway, or by a change in the ubiquitination or degradation of the substrate.

The invention further provides for a method for screening for agents useful in the treatment of a disease or disorder associated with muscle atrophy comprising producing MURF1, MURF3, or MAFBXprotein, and using either of these proteins in in vitro ubiquitin ligase assays. Agents would be screened for their effectiveness in inhibiting ubiquity ligation in vitro.

The invention also provides for a method of treating a disease or disorder in an animal associated with muscle atrophy comprising administering to the animal a compound that modulates the MURF1, MURF3, or MAFBXpathway, ubiquitination, or the synthesis, expression or activity of the MURF1, MURF3, or MAFBXgene or gene product so that symptoms of such disease or disorder are alleviated.

The invention provides for a method of diagnosing a disease or disorder associated with muscle atrophy comprising measuring MURF1, MURF3, or MAFBXgene expression in a patient or patient sample. For example, the invention comprises a method for detecting muscle atrophy in a mammal comprising a) administering to the mammal a composition which comprises a molecule capable of detecting MURF1, MURF3, or MAFBXnucleic acid or polypeptide coupled to an imaging agent; b) allowing the composition to accumulate in the muscle; and c) detecting the accumulated composition so as to detect the presence of MURF1, MURF3, or MA-16 as an indication of muscle atrophy. Such molecules capable of binding or attaching to MURF1, MURF3, or MAFBXmolecules may be, for example, chemicals, nucleic acids, polypeptides, or peptides. In addition, such diagnostics may measure gene expression by directly quantifying the amount of transcript or the amount of expression product. For example, the levels MURF1, MURF3, or MA-61, as well as the proteins encoded there for, may be measured. Such measurements may be made through the use of standard techniques known in the art including but not limited to PCR, Taqman PCR, Northern analysis, Western analysis, or immunohistochemsitry.

The invention further comprises the methods described supra wherein the muscle cells are obtained from a transgenic organism or are within a transgenic organism, wherein the transgenic organism includes, but is not limited to, a mouse, rat, rabbit, sheep, cow or primate.

The invention further comprises a method of inhibiting atrophy in an animal having an atrophy-inducing condition comprising treating the mammal with an effective amount of an inhibitor of MURF1, MURF3, or MAFBXproteins or nucleic acids or treating the cells with an inhibitor of the MURF1, MURF3, or MAFBXpathway. The invention additionally comprises a method of screening compounds useful for the treatment of muscle atrophy and related diseases and disorders comprising contacting a muscle cell expressing MURF1 with a compound and detecting a change in the MURF1, MURF3 OR MAFBX protein activity. The change may measured by PCR, Taqman PCR, phage display systems, gel electrophoresis, yeast-two hybrid assay, Northern or Western analysis, immunohistochemistry, a conventional scintillation camera, a gamma camera, a rectilinear scanner, a PET scanner, a SPECT scanner, a MRI scanner, a NMR scanner, or an X-ray machine. The change in the MURF1, MURF3 OR MAFBX protein activity may also be detected by detecting a change in the interaction of the MURF1, MURF3 OR MAFBX with one or more proteins. This method may be used where the muscle cell is of skeletal origin, is a cultured cell., is obtained from or is within a transgenic organism such as form example a mouse, rat, rabbit, sheep, cow or primate. The change in protein expression may be demonstrated by a change in amount of protein of one or more of the proteins in the ubiquitin pathway.

The invention further comprises a method of inhibiting atrophy in an animal wherein the animal is treated prior to exposure to or onset of the atrophy-inducing condition. Such atrophy-inducing conditions may include immobilization, denervation, starvation, nutritional deficiency, metabolic stress, diabetes, aging, muscular dystrophy, or myopathy. In a preferred embodiment the atrophy inducing condition is immobilization, aging or bed rest. In a preferred embodiment, the atrophy inducing condition is cancer or AIDS.

The invention further comprises a method of causing muscle hypertrophy in skeletal muscle cells comprising treating the cells with an inhibitor of MURF1, MURF3, or MAFBXproteins or nucleic acids or treating the cells with an inhibitor of the MURF1, MURF3, or MAFBXpathway.

In embodiments of the invention that utilize a compound detection system, any detector known in the art, for example, PCR, Taqman PCR, Northern or Western alaysis, immunohistochemistry, a conventional scintillation camera, a gamma camera, a rectilinear scanner, a PET scanner, a SPECT scanner, a MRI scanner, a NMR scanner, and an X-ray machine. In addition, any imaging agent know in the art may be employed, for example, a radionucleotide or a chelate.

The molecules capable of detecting MURF1, MURF3, or MAFBXmay be nucleic acids and mRNA or a synthetic oligonucleotide or a synthetic polypeptide.

In a further embodiment of the invention, patients that suffer from an excess of MURF1, MURF3, or MAFBXmay be treated by administering an effective amount of anti-sense RNA, anti-sense oligodeoxyribonucleotides, or RNAi, corresponding to a MURF1, MURF3, or MAFBXgene coding region, thereby decreasing expression of MURF1, MURF3, and/or MA-61.

### BRIEF DESCRIPTION OF THE FIGURES

FIG. 1: Schematic of MAFBXprotein's association with components of the SCF complex.

FIG. 2: Sequence comparison demonstrating F-box domain of MA-61.

FIG. 3: Schematic of the human MAFBXprotein structural domains.

FIG. 4: Schematic of the human MURF1 protein structural domains.

FIGS. 5A-5B: Sequence comparison between MAFBXand Fbx25 showing broad homology.

FIG. 6: Nucleotide sequence of rat MURF1.

FIG. 7: Deduced amino acid sequence of rat MURF1.

FIGS. 8-8C: Nucleotide sequence of human MURF1.

FIG. 9: Deduced amino acid sequence of human MURF1.

FIG. 10: Nucleotide sequence of rat MAFBX.

FIG. 11: Deduced amino acid sequence of rat MAFBX.

FIG. 12: Nucleotide sequence of human MAFBXclone K8.

FIG. 13: Deduced amino acid sequence of human MAFBXclone K8.

FIG. 14: Sequence comparison demonstrating ring domain of MURF1.

FIG. 15: Schematic of MURF1 protein's association with components of the ubiquitin ligase complex.

FIG. 16: Nucleotide sequence of rat MURF1 VRV splice form.

FIG. 17: Deduced amino acid sequence of rat MURF1 VRV splice form.

FIG. 18: Nucleotide sequence of human MAFBXclone D18.

FIG. 19: Deduced amino acid sequence of human MAFBXclone D18.

FIG. 20: Sequence alignment of rMURF1 with hMURF3.

FIG. 21: Nucleotide sequence of human MURF3 clone C8.

FIG. 22: Deduced amino acid sequence of human MURF3 clone C8.

FIG. 23: The differential display analysis of genes associated with atrophy.

FIG. 24: Northern blots showing the effect of atrophy on expression of muscle creatine kinase (MCK), myoD, myogenin and Myf5.

FIGS. 25:A-25B (FIG. 25A) An immunoblot using antibody raised against full-length rat MuRF1. (FIG. 25B) Northern analysis of MuRF2 and MuRF3

FIG. 26: Sequence alignment of rat and human MAFbx protein, and human Fbx25.

FIGS. 27A-27B: (FIGS. 27A-27BA) Schematic showing the portion of the MAFbx gene to be replaces with the LacZ/PGK neo. (FIGS. 27A-27BB) Schematic showing the portion of the MuRF1 gene to be replaces with the LacZ/PGK neo.

FIGS. 28A-28D (FIGS. 28A-28DA) A time course of rat medial gastrocnemius muscle mass loss was examined in three in vivo models: Denervation, Immobilization and Hindlimb Suspension.

(FIGS. 28A-28DB) Northern blots showing the effect of atrophy on MuRF1 and MAFbx transcripts.

(FIGS. 28A-28DC) Northern blots showing the effect of dexamethasone (DEX) and Interleukin-1 (IL-1) on expression of MuRF1 and MAFbx.

(FIGS. 28A-28DD) Tissue specific expression of MuRF1 and MAFbx.

FIGS. 29A-29D: (FIGS. 29A-29DA) Co-precipitation: MAFbx, Cullin, Skp-1

(FIGS. 29A-29DB) Atrophy induced by over-expression of MAFbx. (FIGS. 29A-29DC) An immunoblot (I.B.) of lysates confirmed the presence of Myc-epitope tagged MAFbx protein in the myotubes infected with the MAFbx virus.

(FIGS. 29A-29DD) Detection of ³²P-labelled high molecular weight ubiquitin conjugates.

FIGS. 30A-30D: (FIGS. 30A-30DA) Confirmation of absence of targeted allele: MAFbx

(FIGS. 30A-30DB) Confirmation of absence of targeted allele: MAFbx

(FIGS. 30A-30DC) Confirmation of absence of targeted allele: MuRF1

(FIGS. 30A-30DD) Confirmation of absence of targeted allele: MuRF1

FIGS. 31A-31C: (FIGS. 31A-31CA) B-gal staining of (MAFbx +/− and MuRF1 +/− tissue in mice.

(FIGS. 31A-31CB) Muscle mass after denervation, as compared to wild type (+/+) mice.

(FIGS. 31A-31CC) Muscle fiber size and variability in muscles from MAFbx deficient mice after denervation.

FIG. 32: Sequence alignment demonstrating that MAFbx protein is the same protein as MA61, and the different names demonstrate a change in nomenclature.

FIG. 33: Sequence alignment demonstrating that MuRF1 protein is the same protein as MA16, and the different names demonstrate a change in nomenclature.

FIG. 34: Sequence alignment of rMA16 with hMURF1.

### DETAILED DESCRIPTION OF THE INVENTION

The invention is based on the Applicant's discovery and characterization of the molecules MURF1, MURF 3, and MA-61. MURF 1 AND MAFBXare expressed in both rat and human adult heart and adult skeletal muscle and their expression is increased under varying conditions of skeletal muscle atrophy. The present invention provides for proteins and nucleic acids of novel human intracellular signaling molecules termed human (h)MURF 1, human (h)MURF 3, and HUMAN MUSCLE ATROPHY-61 (hMA-61) and proteins and nucleic acids of novel rat intracellular signaling molecules termed RAT MURF1, RAT MURF 3, and RAT MUSCLE ATROPHY-61 (rMA-61). Throughout this description, reference to MURF1, MURF 3, or MAFBXproteins and nucleic acids includes, but is not limited to, the specific embodiments of hMURF1, hMURF 3, hMA-61, rMURF1, rMURF 3 or rMAFBXproteins and nucleic acids as described herein. The MURF1 and MURF 3 molecules contain a ring domain and MAFBXcontains an F-box motif. Both of these domains of the molecules facilitate interaction between the molecules, their substrate, and the ubiquitin ligase system.

The present invention relates to novel proteins involved in the ubiquitin pathway and the substrates thereof. The invention provides for novel nucleic acids and polypeptides that are involved in disorders of muscle growth, functioning and proliferation. These include MURF1, MURF 3, or MAFBXproteins or nucleic acids, or domains thereof, having such activity, for example, such as the F-box motif of MA-61, the ring domain of MURF1 or MURF 3, the portion of the MURF3 molecule that co-associates with the Syncoilin gene, and the Fbx25 homology domain of MA-61.

The invention includes MURF1, MURF3, and MAFBXnucleic acids, MURF1, MURF3 and MAFBXpolypeptides, derivatives and analogs thereof, as well as deletion mutants or various isoforms of the MURF1, MURF3, or MAFBXproteins or nucleic acids. They may be provided as fusion products, for example, with non-MURF1, MURF3, or MAFBXpolypeptides and nucleic acids. In addition, the MURF1, MURF3, and MAFBXnucleic acids and peptides may be associated with a host expression system.

The invention further provides for the use of the nucleotides encoding MURF1, MURF3, and MA-61, the proteins, peptides, antibodies to MURF1, MURF3, and MA-61, agonists and antagonists thereof. The invention relates to screening assays designed to identify the substrates of MURF1, MURF3, and MAFBXand/or molecules, which modulate the activity of the novel molecules MURF1, MURF3, and MAFBXindependently or in relation to the substrates thereof. In addition, the invention relates to the use of screening assays used to identify potential therapeutic agents which inhibit, block or ameliorate muscle atrophy and related diseases and disorders.

Genes

The invention provides for the nucleic acid molecules, which encode MURF1, MURF3, or MA-61. The invention includes the nucleic acid sequences encoding polypeptides or peptides which correspond to MURF1, MURF3 and MAFBXgene products, including the functional domains of MURF1, MURF3 and MA-61, such as for example the F-box motif of MA-61, the ring domain of MURF1 or MURF3, the portion of the MURF3 molecule that co-associates with the Syncoilin gene, and the Fbx25 homology domain of MA-61, or derivatives, fragments, or domains thereof, mutated, truncated or deletion forms thereof, and host cell expression systems incorporating or producing any of the aforementioned.

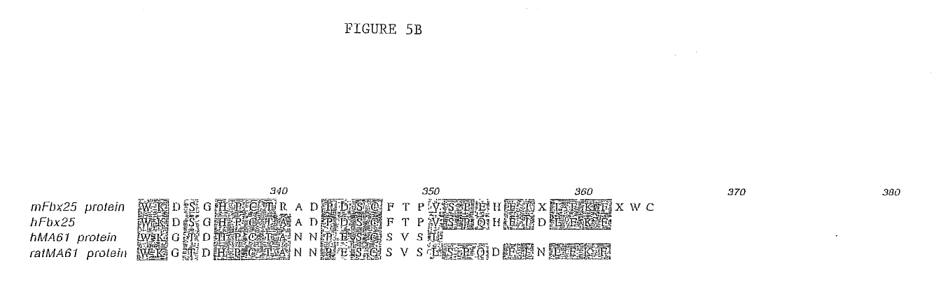

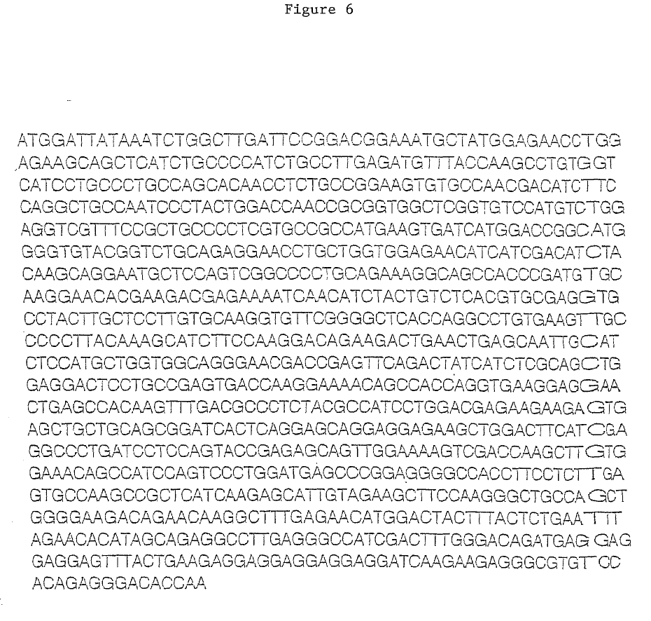

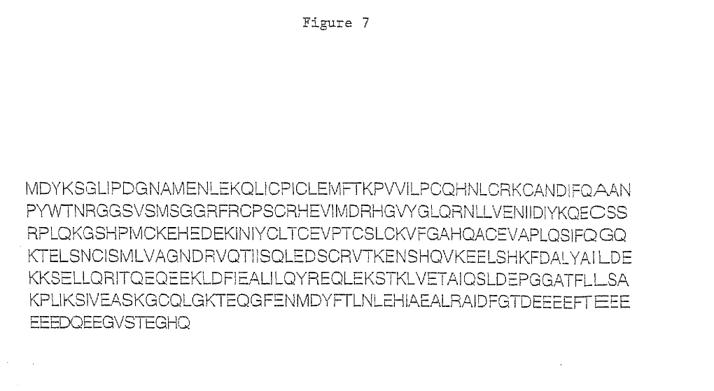

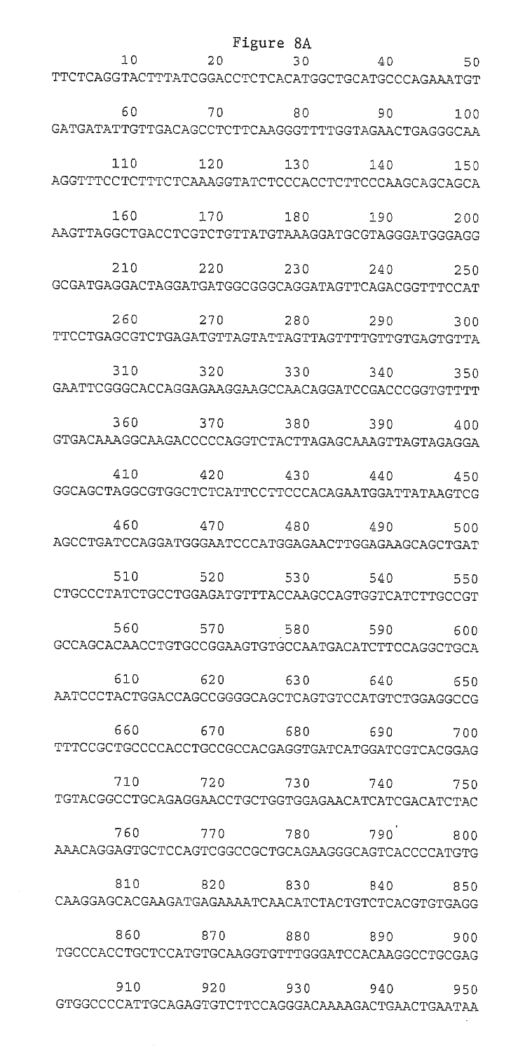

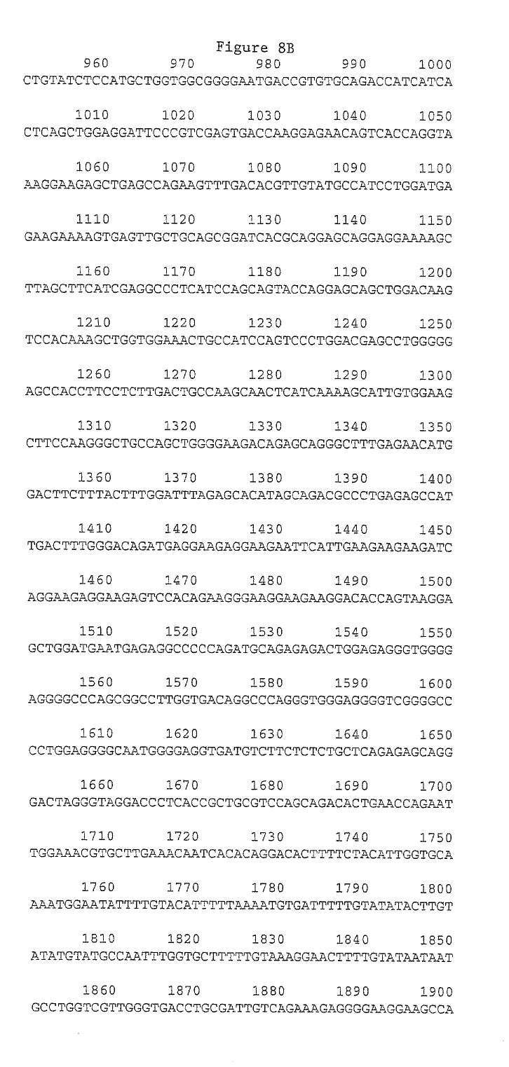

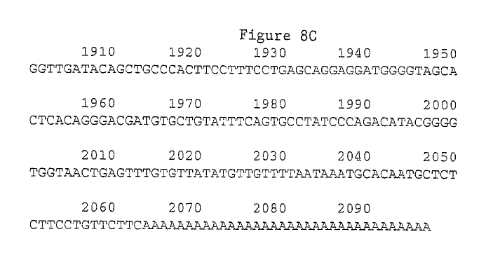

The invention includes the nucleic acid molecules containing the DNA sequences in FIGS. 6, 8(a-c), 10, 12,16, 18, and 21; any DNA sequence that encodes a polypeptide containing the amino acid sequence of FIGS. 7, 9, 11, 13,17, and 19; any nucleotide sequence that hybridizes to the complement of the nucleotide sequences that encode the amino acid sequence of FIGS. 6, 8(a-c), 10, 12,16, 18, and 2 under stringent or highly stringent conditions, and/or any nucleotide sequence that hybridizes to the complement of the nucleotide sequence that encodes the amino acid sequence of FIGS. 7, 9, 11, 13,17, 19, and 22 under less stringent conditions.

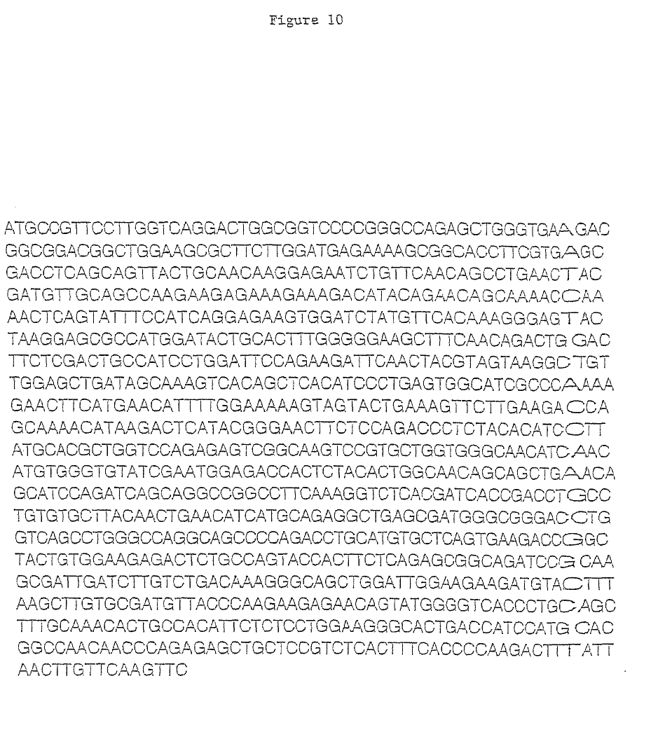

In a specific embodiment, the nucleotide sequences of the present invention encompass any nucleotide sequence derived from a mammalian genome which hybridizes under stringent conditions to FIGS. 10, 12, and 18 and encodes a gene product which contains either an F-box motif and is at least 47 nucleotides in length.

The invention includes nucleic acid molecules and proteins derived from mammalian sources. The nucleic acid sequences may include genomic DNA, cDNA, or a synthetic DNA. When referring to a nucleic acid that encodes a particular amino acid sequence, it should be understood that the nucleic acid may be a cDNA sequence from which an mRNA species is transcribed that is processed to encode a particular amino acid sequence.

The invention also includes vectors and host cells that contain any of the disclosed sequences and/or their complements, which may be linked to regulatory elements. Such regulatory elements may include but are not limited to promoters, enhancers, operators and other elements known to those skilled in the art to drive or regulate expression, for example CMV, SV40, MCK, HSA, and adeno promoters, the lac system, the trp system, the TRC system, promoters and operators of phage A.

The invention further includes fragments of any of the nucleic acid sequences disclosed herein and the gene sequences encoding MURF1, MURF3, and MAFBXgene products that have greater than about 50% amino acid identity with the disclosed sequences.

In specific embodiments, the invention provides for nucleotide fragments of the nucleic sequences encoding MURF1, MURF3, and MAFBX(FIGS. 6, 8(a-c), 10, 12,16, 18, and 21). Such fragments consist of at least 8 nucleotides (i.e. hybridization portion) of an MURF1, MURF3, or MAFBXgene sequence; in other embodiments, the nucleic acids consists of at least 25 continuous nucleotides, 50 nucleotides, 100 nucleotides, 150 nucleotides, 150 nucleotides, or 200 nucleotides of an MURF1, MURF3, or MAFBXsequence. In another embodiment the nucleic acids are smaller than 47 nucleotides in length. The invention also relates to nucleic acids hybridizable or complementary to the foregoing sequences. All sequences may be single or double stranded. In addition, the nucleotide sequences of the invention may include nucleotide sequences that encode polypeptides having at least 30%, 35%, 40%, 45%, 50%, 55%, 60%, 65%, 70%, 75%, 80%, 85%, 90%, 95%, 98%, or higher amino acid sequence identity to the polypeptides encoded by the MURF1, MURF3, or MAFBXsequences of FIGS. 7, 9, 11, 13,17, and 19.

One embodiment of the invention is a recombinant nucleic acid encoding MURF1, MURF3, or MAFBXpolypeptide which corresponds to the amino acid sequence as set forth herein in FIGS. 7, 9, 11, 13,17, and 1 or a fragment thereof having MURF1, MURF3, or MA-61-specific activity or expression level.

Still another embodiment is an isolated nucleic acid comprising a nucleotide sequence as set forth herein in FIGS. 6, 8 (a-c), 10, 12,16, 18, and 21 or a fragment thereof having at least 18 consecutive bases and which can specifically hybridize with the complement of a nucleic acid having the sequence of native MURF1 or MAFBX.

Further, the sequence of the disclosed MURF1, MURF3, or MAFBX nucleic acids may be optimized for selected expression systems (Holler, et al., (1993) Gene 136:323-328; Martin, et al., (1995) Gene 154:150-166) or used to generate degenerate oligonucleotide primers and probes for use in the isolation of natural MURF1, MURF3, or MAFBX encoding nucleic acid sequences (“GCG” software, Genetics Computer Group, Inc., Madison, Wis.). MURF1, MURF3, or MAFBX encoding nucleic acids may be part of expression vectors and may be incorporated into recombinant host cells, e.g., for expression and screening, for transgenic animals, or for functional studies such as the efficacy of candidate drugs for diseases associated with MURF1 or MA-61-mediated cellular activity or MURF1, MURF3, or MAFBX mRNA and/or protein expression. Expression systems are selected and/or tailored to effect MURF1, MURF3, or MAFBXpolypeptide structural and functional variants through alternative post-translational processing.

The claimed MURF1, MURF3, or MAFBXnucleic acids may be isolated or pure, and/or are non-natural. A “pure” nucleic acid constitutes at least about 90%, and preferably at least about 99% by weight of the total nucleic acid in a given sample. A “non-natural” nucleic acid is one that has been manipulated to such an extent that it may not be considered a product of nature. One example of a non-natural nucleic acid is one produced through recombinant techniques known in the art. The subject nucleic acids may be synthesized, produced by recombinant technology, or purified from cells. Nucleic acids comprising the nucleotide sequence disclosed herein and fragments thereof, may contain such sequences or fragments at a terminus, immediately flanked by a sequence other than that to which it is joined on a natural chromosome, or flanked by a native flanking region fewer than 10 kb, preferably fewer than 2 kb, which is immediately flanked by a sequence other than that to which it is joined on a natural chromosome. While the nucleic acids are usually the RNA or DNA sequences, it is often advantageous to use nucleic acids comprising other bases or nucleotide analogs to provide, example, modified stability.

The invention provides a wide variety of applications for MURF1, MURF3, or MAFBXnucleic acids including but not limited to identifying and studying molecules, agents and drugs that modulate muscle atrophy, ubiquitination, or the expression or activity of MURF1, MURF3, and MAFBXnucleic acids or polypeptides themselves; as markers of muscle atrophy or ubiquitination; as markers for the prevention or reduction of muscle atrophy or ubiquitination; identifying and studying molecules, agents and drugs that modulate muscle dystrophy; as markers of muscle dystrophy; as markers for the prevention or reduction of muscle dystrophy; as translatable transcripts, hybridization probes, PCR primers, or diagnostic nucleic acids, imaging agents; detecting the presence of MURF1, MURF3, or MAFBXgenes and gene transcripts; and detecting or amplifying nucleic acids encoding additional MURF1, MURF3, or MAFBXhomologs and structural analogs.

Novel agents that bind to or modulate the expression of MURF1, MURF3, or MAFBXmRNA described herein may prevent muscle atrophy in cells expressing MURF1, MURF3, or MAFBXmRNA. Novel agents that bind to or modulate the activity of MURF1, MURF3, or MA-61-mediated ubiquitination described herein may prevent muscle atrophy in cells containing either the MURF1, MURF3, or MAFBXproteins. Drugs or agents which inhibit the expression of MAFBXmRNA, or the activity of MAFBXproteins, or inhibit the MA61 pathway, are predicted to decrease specific SCF E3 ubiquitin ligase-mediated ubiquitination of protein targets. Drugs or agents which inhibit the expression of MURF1, MURF3, mRNA, or the activity of MURF1 or MURF3 proteins, or inhibit the MURF1 or MuRF3 pathway, are predicted to decrease specific ring-domain-mediated ubiquitination of protein targets. Rugs or agents which inhibit the expression of MA61 mRNA or the activity of MAFbx proteins are predicted to decrease F-box mediated ubiquitination of protein targets. Dominant negative, inhibitory forms of MURF1, MURF3, or MAFBXcDNA or genomic DNA may be used in gene therapy to block skeletal muscle atrophy. Dominant negative inhibitory forms of MURF1, MURF3, or MAFBXcDNA or genomic DNA, in which either the F-box domain or the Fbx25 homology domain of MA-61, or the ring domain of MURF1 or MURF3 are expressed alone, may also be used in gene therapy to block skeletal muscle atrophy.

The invention additionally encompasses antibodies, antagonists, agonists, compounds, or nucleotide constructs that inhibit expression of the MURF1, MURF3, and MAFBXgenes (including for example transcription factor inhibitors, antisense and ribozyme molecules, and gene or regulatory sequence replacement constructs) or that promote expression of dominant-negative forms of MURF1, MURF3, or MAFBX(including for example expression constructs in which the coding sequences are operatively linked with expression control elements).

The invention provides for the detection of nucleic acids encoding MURF1, MURF3, and MA-61. This may be done through the use of nucleic acid hybridization probes and replication/amplification primers having a MURF1, MURF3, or MAFBXcDNA-specific sequence and sufficient to effect specific hybridization with FIGS. 6, 8(a-c), 10, 12,16, 18, and 21. Demonstrating specific hybridization generally requires stringent conditions, for example, hybridizing in a buffer comprising 30% formamide in 5×SSPE (0.18 M NaCl, 0.01 M NaPO₄, pH 7.7, 0.001 M EDTA) buffer at a temperature of 42° C. and remaining bound when subject to washing at 42° C. with 0.2×SSPE; preferably hybridizing in a buffer comprising 50% formamide in 5×SSPE buffer at a temperature of 42° C. and remaining bound when subject to washing at 42° C. with 0.2×SSPE buffer at 42° C., or most preferably hybridizing in a buffer comprising 20% SDS, 10% BSA, 1M NaPO₄, 0.5M EDTA, pH 8 at a temperature of 60° C. and remaining bound when subject to washing at 65° C. with 2×SSC, 0.1% SDS. MURF1 or MAFBXcDNA homologs can also be distinguished from one another using alignment algorithms, such as BLASTX (Altschul, et al., (1990) Basic Local Alignment Search Tool, J. Mol. Biol. 215:403-410).

Also encompassed is the use of the disclosed sequences to identify and isolate gene sequences present at the same genetic or physical location as the sequences herein disclosed, and such sequences can, for example, be obtained through standard sequencing and bacterial artificial chromosome (BAC) technologies. Also encompassed is the use of the disclosed sequences to clone gene homologues in human or other species. To do so, the disclosed sequences may be labeled and used to screen a cDNA or genomic library. The level of stringency required will depend on the source of the DNA used. Thus low stringency conditions may be appropriate in certain circumstances, and such techniques are well know in the art. (See e.g. Sambrook, et al., 1989, Molecular Cloning, A Laboratory Manual, Second Edition, Cold Spring Harbor Press, N.Y.) In addition, a MURF1, MURF3, or MAFBXhomologue may be isolated with PCR by using two degenerate oligonucleotide primer pools designed using the sequences disclosed herein. The identified fragment may then be further used to isolate a full length clone by various techniques known in the art, including the screening of a cDNA or genomic library. In addition, PCR may be used to directly identify full length cDNA sequences (see e.g. Sambrook et al, supra). The disclosed sequences may also be used to identify mutant MURF1, MURF3, and MAFBXalleles. Mutant alleles are used to generate allele-specific oligonucleotide (ASO) probes for high-throughput clinical diagnoses. MURF1, MURF3, and MAFBXalleles may be identified by a number of techniques know in the art including but not limited to single strand conformation polymorphism (SSCP) mutation detection techniques, Southern blotting, and/or PCR amplification techniques.

MURF1, MURF3, or MAFBXnucleic acids are also used to modulate cellular expression or intracellular concentration or availability of MURF1, MURF3, or MAFBXpolypeptides. MURF1, MURF3, or MAFBXinhibitory nucleic acids are typically antisense—single stranded sequences comprising complements of the disclosed MURF1, MURF3, or MAFBXcoding sequences. Antisense modulation of the expression of a given MURF1, MURF3, or MAFBXpolypeptide may employ antisense nucleic acids operably linked to gene regulatory sequences. Cells are transfected with a vector comprising a MURF1, MURF3, or MAFBXsequence with a promoter sequence oriented such that transcription of the gene yields an antisense transcript capable of binding to endogenous MURF1, MURF3, or MAFBXencoding mRNA. Transcription of the antisense nucleic acid may be constitutive or inducible and the vector may provide for stable extrachromosomal maintenance or integration. Alternatively, single-stranded antisense nucleic acids that bind to genomic DNA or mRNA encoding a given MURF1, MURF3, or MAFBXpolypeptide may be administered to the target cell at a concentration that results in a substantial reduction in expression of the targeted polypeptide. An enhancement in MURF1, MURF3, or MAFBXexpression or activity is effected by introducing into the targeted cell type MURF1, MURF3, or MAFBXnucleic acids which increase the functional expression of the corresponding gene products. Such nucleic acids may be MURF1, MURF3, or MAFBXexpression vectors, vectors which upregulate the functional expression of an endogenous allele, or replacement vectors for targeted correction of mutant alleles. Techniques for introducing the nucleic acids into viable cells are known in the art and include, but are not limited to, retroviral-based transfection or viral coat protein-liposome mediated transfection.

Proteins and Peptides

The invention provides for polypeptides or peptides which correspond to MURF1, MURF3, and MAFBXgene products, including the functional domains of MURF1, MURF3, and MA-61, such as for example the F-box motif of MA-61, the ring domain of MURF1 or MURF3, the portion of the MURF3 molecule that co-associates with the Syncoilin gene, and the Fbx25 homology domain of MA-61, or derivatives, is fragments, or domains thereof, mutated, truncated or deletion forms thereof, fusion proteins thereof, and host cell expression systems incorporating or producing any of the aforementioned.

One embodiment of the invention is an isolated MURF1, MURF3 or MAFBXpolypeptide comprising the amino acid sequence as set forth herein in FIGS. 7, 9, 17, 11, 13, 19, and 22, or a fragment thereof having MURF1, MURF3 or MA-61-specific activity or expression levels.

The sequences of the disclosed MURF1, MURF3, or MAFBXpolypeptide sequences are deduced from the MURF1, MURF3, or MAFBXnucleic acids. The claimed MURF1, MURF3, or MAFBXpolypeptides may be isolated or pure, and/or are non-natural. An “isolated” polypeptide is one that is no longer accompanied by some of the material with which it is associated in its natural state, and that preferably constitutes at least about 0.5%, and more preferably at least about 5% by weight of the total polypeptide in a given sample. A “pure” polypeptide constitutes at least about 90%, and preferably at least about 99% by weight of the total polypeptide in a given sample. The subject polypeptides may be synthesized, produced by recombinant technology, or purified from cells. A “non-natural” polypeptide is one that has been manipulated to such an extent that it may no longer be considered a product of nature. One example of a non-natural polypeptide is one produced through recombinant techniques known in the art. A wide variety of molecular and biochemical methods are available for biochemical synthesis, molecular expression and purification of the subject compositions (see e.g., Molecular Cloning, A Laboratory Manual, Sambrook, et al., Cold Spring Harbor Laboratory, Cold Spring Harbor, N.Y.; Current Protocols in Molecular Biology (Eds. Ausubel, et al., Greene Publ. Assoc., Wiley-Interscience, NY).

The invention also provides for the use of polypeptides or peptides which correspond to functional domains of MURF1, MURF3, and MA-61, such as for example the F-box motif of MA-61, the ring domain of MURF1 or MURF3, the portion of the MURF3 molecule that co-associates with the Syncoilin gene, and the Fbx25 homology domain of MA-61, or derivatives, fragments, or domains thereof, mutated, truncated or deletion forms thereof, fusion proteins thereof, and host cell expression systems incorporating or producing any of the aforementioned to screen or agents that interact with or modify any of these molecules, muscle atrophy and related disorders and diseases. The screening of molecules may be accomplished by any number of methods known in the art including but are not limited to immunoprecipitation, size fractionization, Western blot, and gel electrophoresis. Preferably the method of screening is a yeast two-hybrid system, or any variation thereof. The invention encompasses both in vitro and in vivo tests, which may screen small molecules, large molecules, compounds, recombinant proteins, peptides, nucleic acids and antibodies.

A number of applications for MURF1, MURF3 or MAFBXpolypeptides, or peptide fragments, are suggested from their properties. They may be useful for identifying and studying molecules, agents and drugs that modulate muscle atrophy, muscle dystrophy, ubiquitination, or the expression or activity of MURF1, MURF3 and MAFBXthemselves. They may be useful as markers of muscle atrophy, muscle dystrophy, or ubiquitination, and as markers for the prevention or reduction of muscle atrophy, muscle dystrophy, or ubiquitination. They may be used for the generation of antibodies as well.

In addition, these disclosed polypeptides and nucleic acids may be useful in inhibiting muscle atrophy, muscle dystrophy, the MURF1, MURF3, and MAFBXpathway, or ubiquitination. In addition, they may be useful in treating conditions associated with muscle atrophy, muscle dystrophy, or increased ubiquitination. MURF1, MURF3 or MAFBXpolypeptides may be useful in the study, treatment or diagnosis of conditions similar to those which are treated using growth factors, cytokines and/or hormones. Functionally equivalent MURF1, MURF3 and MAFBXgene products may contain deletions, additions, and/or substitutions. Such changes may result in no functional change in the gene product, or the gene product may be engineered to product alterations in the gene product. Such gene products may be produced by recombinant technology through techniques known in the art, such as in vitro recombinant DNA techniques, synthetic techniques, and in vivo genetic recombination (see e.g. Sambrook, et al., supra). In addition, RNA which encodes such gene products may be synthesized chemical using techniques know in the art (see, e.g. “Oligonucleotide Synthesis”, 1984 Gait, ed., IRL Press, Oxford.)

Antibodies

The present invention also provides for antibodies to the MURF1, MURF3 or MAFBXpolypeptides described herein which are useful for detection of the polypeptides in, for example, diagnostic applications. For preparation of monoclonal antibodies directed toward MURF1, MURF3 or MAFBXpolypeptides, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in “Monoclonal Antibodies and Cancer Therapy”, Alan R. Liss, Inc. pp. 77-96) and the like are within the scope of the present invention.

The monoclonal antibodies for diagnostic or therapeutic use may be human monoclonal antibodies or chimeric human-mouse (or other species) monoclonal antibodies. Human monoclonal antibodies may be made by any of numerous techniques known in the art (e.g., Teng et al., 1983, Proc. Natl. Acad. Sci. U.S.A. 80:7308-7312; Kozbor et al., 1983, Immunology Today 4:72-79; Olsson et al., 1982, Meth. Enzymol. 92:3-16). Chimeric antibody molecules may be prepared containing a mouse antigen-binding domain with human constant regions (Morrison et al., 1984, Proc. Natl. Acad. Sci. U.S.A. 81:6851, Takeda et al., 1985, Nature 314:452).

Various procedures known in the art may be used for the production of polyclonal antibodies to the MURF1, MURF3 or MAFBXpolypeptides described herein. For the production of antibody, various host animals can be immunized by injection with the MURF1, MURF3, or MAFBXpolypeptides, or fragments or derivatives thereof, including but not limited to rabbits, mice and rats. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, polypeptides, oil emulsions, keyhole limpet hemocyanins, dinitrophenol, and potentially useful human adjuvants such as BCG (Bacille Calmette-Guerin) and Corynebacterium parvum.

A molecular clone of an antibody to a selected MURF1, MURF3, or MAFBXpolypeptide epitope can be prepared by known techniques. Recombinant DNA methodology (see e.g., Maniatis et al., 1982, Molecular Cloning, A Laboratory Manual, Cold Spring Harbor Laboratory, Cold Spring Harbor, N.Y.) may be used to construct nucleic acid sequences which encode a monoclonal antibody molecule, or antigen binding region thereof.

The present invention provides for antibody molecules as well as fragments of such antibody molecules. Antibody fragments which contain the idiotype of the molecule can be generated by known techniques. For example, such fragments include, but are not limited to, the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent. Antibody molecules may be purified by known techniques including, but not limited to, immunoabsorption or immunoaffinity chromatography, chromatographic methods such as HPLC (high performance liquid chromatography), or a combination thereof.

The invention also provides for single chain Fvs. A single chain Fv (scFv) is a truncated Fab having only the V region of a heavy chain linked by a stretch of synthetic peptide to a V region of a light chain. See, for example, U.S. Pat. Nos. 5,565,332; 5,733,743; 5,837,242; 5,858,657; and 5,871,907 assigned to Cambridge Antibody Technology Limited incorporated by reference herein.

Assays

The subject MURF1, MURF3 and MAFBXnucleic acids, polypeptides, and antibodies which bind MURF1, MURF3, and MAFBXpolypeptides find a wide variety of uses including but not limited to use as immunogens; targets in screening assays; and bioactive reagents for modulating, preventing, detecting or measuring muscle atrophy or ubiquitination. The molecules listed supra may be introduced, expressed, or repressed in specific populations of cells by any convenient way, including but not limited to, microinjection, promoter-specific expression of recombinant protein or targeted delivery via lipid vesicles.

One aspect of this invention provides methods for assaying and screening for substrates, and fragments, derivatives and analogs thereof, of MURF1, MURF3 and MAFBXgenes and gene products and to identify agents that interact with MURF1, MURF3, and MAFBXgenes and gene products. The invention also provides screening assays to identify compounds that modulate or inhibit the interaction of MURF1, MURF3 and MAFBXgenes and gene products with their substrates and/or subunits of the ubiquitin ligase complex. The screening assays of the present invention also encompass high-throughput screening assays to identify modulators of MURF1, MURF3, and MAFBXgene and gene product expression and activity. Such assays may identify agonists or antagonists of the MURF1, MURF3 or MAFBXgene products.

The invention provides screening methods for identification of agents that bind to or directly interact with MURF1, MURF3, and MAFBXgenes and gene products. Such screening methodologies are well known in the art (see, e.g. PCT International Publication No. WO 96/34099, published Oct. 31, 1996). The agents include both endogenous and exogenous cellular components. These assays may be performed in vitro, or in intact cells in culture or in animal models.

In a preferred embodiment, a yeast two hybrid assay system is used to determine substrates, and fragments, derivatives and analogs thereof, of MURF1, MURF3, and MAFBXgenes and to identify agents that interact with MURF1, MURF3 and MAFBXgene products (Fields and Song, 1989, Nature 340:245-246 and U.S. Pat. No. 5,283,173). The system is based on the detection of expression of a reporter gene, the transcription of which is dependent on the reconstitution of a transcriptional regulator by the interaction of two proteins, each fused to one half of the transcriptional regulator. MURF1, MURF3, and MAFBXproteins or derivatives thereof and the proteins to be tested are expressed as fusion proteins to a DNA binding domain, and to a transcriptional regulatory domain.

The invention provides MURF1, MURF3 or MA-61-specific binding agents, methods of identifying and making such agents, and their use in diagnosis, therapy and pharmaceutical development. MURF1, MURF3, or MA-61-specific binding agents include MURF1, MURF3 or MA-61-specific antibodies and also includes other binding agents identified with assays such as one-, two- and three-hybrid screens, and non-natural binding agents identified in screens of chemical libraries such as described below (see, e.g., Harlow and Lane (1988) Antibodies, A Laboratory Manual, Cold Spring Harbor Laboratory, Cold Spring Harbor, N.Y., for a discussion of manufacturing and using antibodies). Agents of particular interest modulate MURF1, MURF3 or MAFBXmRNA or polypeptide function, activity or expression.

The invention provides efficient methods of identifying agents, compounds or lead compounds for agents active at the level of MURF1, MURF3 or MAFBXmodulatable cellular function or mRNA or polypeptide expression. Generally, these screening methods involve assaying for compounds which modulate the interaction of MURF1, MURF3 or MAFBXpolypeptide or nucleic acid with a natural MURF1, MURF3 or MAFBXbinding target or assaying for compounds which modulate the expression of MURF1, MURF3 or MAFBXmRNA or polypeptide. A wide variety of assays for binding agents or agents that modulate expression are provided including, but not limited to, protein-protein binding assays, immunoassays, or cell based assays. Preferred methods are amenable to automated, cost-effective, high throughput screening of chemical libraries for lead compounds.

In vitro binding assays employ a mixture of components including a MURF1, MURF3, or MAFBXpolypeptide, which may be part of a fusion product with another peptide or polypeptide, e.g. a tag for detection or anchoring. The assay mixtures comprise a natural MURF1, MURF3, or MAFBXbinding target. While native binding targets may be used, it is frequently preferred to use portions thereof as long as the portion provides binding affinity and avidity to the subject MURF1, MURF3 or MAFBXconveniently measurable in the assay. The assay mixture also comprises a candidate pharmacological agent. Candidate agents encompass numerous chemical classes, though typically they are organic compounds, preferably small organic compounds, and are obtained from a wide variety of sources including libraries of synthetic or natural compounds. A variety of other reagents such as salts, buffers, neutral proteins, (e.g., albumin,) detergents, protease inhibitors, nuclease inhibitors, or antimicrobial agents may also be included. The mixture components can be added in any order that provides for the requisite bindings and incubations may be performed at any temperature which facilitates optimal binding. The mixture is incubated under conditions whereby, but for the presence of the candidate pharmacological agent, the MURF1, MURF3 or MAFBXpolypeptide specifically binds the binding target, portion or analog with a reference binding affinity. Incubation periods are chosen for optimal binding but are also minimized to facilitate rapid, high throughput screening.

After incubation, the agent-based binding between the MURF1. MURF3 or MAFBXpolypeptide and one or more binding targets is detected by any convenient way. For cell-free binding type assays, a separation step is often used to separate bound from unbound components. Separation may be effected by any number of methods that include, but are not limited to, precipitation or immobilization followed by washing by, e.g., membrane filtration or gel chromatography. For cell-free binding assays, one of the components usually comprises or is coupled to a label. The label may provide for direct detection as radioactivity, luminescence, optical or electron density, or indirect detection such as an epitope tag or an enzyme. A variety of methods may be used to detect the label depending on the nature of the label and other assay components, including but not limited to, through optical or electron density, radiative emissions, nonradiative energy transfers, or indirectly detected with, as a nonlimiting example, antibody conjugates. A difference in the binding affinity of the MURF1, MURF3 or MAFBXpolypeptide to the target in the absence of the agent as compared with the binding affinity in the presence of the agent indicates that the agent modulates the binding of the MURF1, MURF3 or MAFBXpolypeptide to the corresponding binding target. A difference, as used herein, is statistically significant and preferably represents at least a 50%, more preferably at least a 90% difference.

The invention further provides for a method for screening for agents useful in the treatment of a disease or disorder associated with muscle atrophy comprising contacting a cell expressing MURF1, MURF3 or MAFBXhaving the amino acid sequence of FIGS. 7. 9. 17, 11, 13, 19, and 22, respectively, or a fragment thereof, and its substrate, with a compound and detecting a change in the activity of either MURF1, MURF3 or MAFBXgene products. Such change in activity may be manifest by a change in the interaction of MURF1, MURF3 or MAFBXgene products with one or more proteins, such as one of their substrates or a component of the ubiquitin pathway, or by a change in the ubiquitination or degradation of the substrate.

MURF1, MURF3 or MA-61-specific activity, function or expression may be determined by convenient in vitro cell based or in vivo assays. In vitro or cell based assays include but are not limited to binding assays and cell culture assays and ubiquitination assays In vivo assays include but are not limited to immune response, gene therapy and transgenic animals and animals undergoing atrophy. Binding assays encompass any assay where the specific molecular interaction of MURF1, MURF3 or MAFBXpolypeptide with a binding target is evaluated or where the mRNA or protein expression level or activity of MURF1, MURF3, or MAFBXis evaluated or the binding or ubiquitination of a substrate is evaluated. The binding target may be, for example, a phosphorylated protein, a specific immune polypeptide such as an antibody, or a MURF1, MURF3 or MA-61-nucleic acid-specific binding agent, such as, for example, and anti-sense oligonucleotide. Potential binding targets for MURF1, MURF3 and MAFBXnucleic acids and polypeptides include other known members of the SCF E3 ubiquitin ligase complex and the dystrophin protein complex. For example, it is known that other F-box containing proteins bind to a protein called Cullin-1, or a family member of the Cullin family, such as Cullin -2, Cullin-3, Cullin-4a, Cullin-4b or Cullin-5 (Lisztwan J, Marti A, Sutterluty H, Gstaiger M, and Wirbelauer C, Krek W, 1998 EMBO 17(2):368-83; Lyapina S A, Correll C C, Kipreos E T, Deshaies R J., 1998 Proc Natl Acad Sci USA 95(13):7451-6.) Therefore, one potential assay would be to see if a test compound could disrupt binding of MAFBXto a Cullin family member. Also, F-box proteins which are part of SCF E3 ubiquitin ligase complexes are known to bind Skp-1, or Skp-1 family members (Skowyra, et al, 1997, Cell 91:209-219). Therefore, a potential assay would be to determine if a test compound could disrupt binding of MAFBXto Skp-1 or a Skp-1 family member. Further, F-box proteins which are part of SCF E3 ubiquitin ligase complex bind phosphorylated substrates, which are then ubiquitinated. (Skowyra, et al, 1997, Cell 91:209-219). So, in a featured embodiment of this invention, a potential assay would be to determine if a test compound could disrupt binding of MAFBXprotein to a phosphorylated substrate, or to determine if a test compound could decrease MA-61-mediated ubiquitination of a phosphorylated substrate.

The finding that MURF3 protein associates with a member of the dystrophin complex suggests that inhibition of MURF3 protein or nucleic acids could stabilize the complex, thus helping to treat muscular dystrophy, and other conditions in which the dystrophin complex is subjected to ubiquitin-mediated degradation. Thus another embodiment of this invention is the use of MURF1, MURF3 or MA-61or other molecules involved in their pathways, and especially inhibitors thereof, in the inhibition of the MURF1, MURF3, or MAFBXpathway or treatment of muscular dystrophy and symptoms, conditions and diseases associated with defects in the neuromuscular junction.

The MURF1, MURF3 or MAFBXcDNAs, or antibodies which recognize MURF1, MURF3 or MAFBXpolypeptides, may be useful as diagnostic tools, such as through the use of oligonucleotides as primers in a PCR test to amplify those sequences having similarities to the oligonucleotide primer, and to see how much MURF1, MURF3 or MAFBXmRNA is present in a particular tissue or sample under normal and non-normal, for example, atrophying conditions, or determination of up-regulation of MURF1, MURF3 or MAFBXproteins, by immunostaining with antibodies, or by an ELISA test with antibodies. The isolation of MURF1, MURF3 or MAFBXprovides the key to studying their properties and designing assays for agents that interact with or alter the expression or activity of these molecules, or their pathway. The isolation of MURF1, MURF3 or MAFBXalso provides the key to developing treatments for conditions in which MURF1, MURF3 or MAFBXexpression or activity is disrupted.

The invention also provides for a method of diagnosing a disease or disorder associated with muscle atrophy comprising measuring MURF1, MURF3, or MAFBXgene expression in a patient sample. For example, the invention comprises a method for detecting muscle atrophy in a mammal comprising a) administering to the mammal a composition which comprises a molecule capable of detecting MURF1, MURF3 or MAFBXnucleic acid or polypeptide coupled to an imaging agent; b) allowing the composition to accumulate in the muscle; and c) detecting the accumulated composition so as to image the muscle atrophy. In addition, MURF1, MURF3, and MAFBXcould be detected using mRNA or protein obtained from a subject and using standard methodology such as PCRT, Northern analysis, Western analysis, ELISA, or immunostaining.

Suitable imaging agents that can be coupled to MURF1, MURF3 or MAFBXnucleic acid or polypeptide for use in detection include, but are not limited to, agents useful in magnetic resonance imaging (MRI) such as gadolinium chelates (see for example Ladd, D L, et al., 1999, Bioconjug Chem 10:361-370), covalently linked nonionic, macrocyclic, multimeric lanthanide chelates (see for example Ranganathan, RS, et al., 1998, Invet Radiol 33:779-797), and monoclonal antibody-coated magnetite particles (see To, S Y, et al., 1992, J Clin Laser Med Surg 10:159-169). For reviews relating to basic principles of MRI see Kirsch, J E, 1991, Top Magn Reson Imaging 3:1-18 and Wallis, F and Gilbert, F J, 1999, J R Coll Surg Edinb 44:117-125. Radionucleotides are also suitable imaging agents for use in nuclear medicine techniques such as positron emission tomography (PET), single positron emission computed tomography (SPECT), and computerized axial tomography (CAT) scans. By way of non-limiting example, such agents include technetium 99 m, gallium 67 citrate, iodine 123 and indium 111 (see Coleman, R E, 1991, Cancer 67:1261-1270). Other radionucleotides suitable as imaging agents include ¹²³I and ¹¹¹In-DTPA (see Kaltsas, G A, et al., 1998, Clin Endocrinol (Oxf) 49:685-689), radiolabeled antibodies (see Goldenberg, D M and Nabi, H A, 1999, Semin Nucl Med 29:41-48 and Steffens, M G, et al., 1999, J Nucl Med 40:829-836). For reviews relating to basic principles of radionuclear medicine techniques, see Schiepers, C. And Hoh, C K, 1998, Eur Radiol 8:1481-1494 and Ferrand, S K, et al., 1999, Surg Oncol Clin N Am 8:185-204. Any imaging agent may be utilized, including, for example, a radionucleotide or a chelate.

The disclosed methods may be applicable in vivo or in vitro, and the cells may include, for example, cultured muscle cells, myoblasts, C2C12 cells, differentiated myoblasts, or myotubes.

The invention also provides for a method of treating a disease or disorder in an animal associated with muscle atrophy comprising administering to the animal a compound that modulates the synthesis, expression or activity of the MURF1, MURF3 or MAFBXgene or gene product so that symptoms of such disease or disorder are alleviated.

(For a detailed explanation of other assays and methodologies for use of the invention herein described, see also PCT International Publication No. WO 00/12679, published Mar. 9, 2000, which is incorporated by reference herein in its entirety).

The invention also relates to host cells and animals genetically engineered to express MURF1, MURF3 or MAFBXpolypeptides or peptides which correspond to functional domains of MURF1, MURF3 and MA-61, such as for example the F-box motif of MA-61, the ring domain of MURF1 and MURF 3, the portion of the MURF3 molecule that co-associates with the Syncoilin gene, and the Fbx25 homology domain of MA-61, or derivatives, fragments, or domains thereof, mutated, truncated or deletion forms thereof, fusion proteins thereof, and host cell expression systems incorporating or producing any of the aforementioned, as well as host cells and animals genetically engineered to inhibit or “knock-out” expression of the same. Animals of any species, including but not limited to mice, rats, rabbits, guinea pigs, pigs, goats, sheep, and non-human primates, may be used to generate transgenic animals and their progeny, wherein “transgenic” means expressing gene sequences from another source, for example another species, as well as over-expressing endogenous MURF1, MURF3 or MAFBXsequences, or non-expression of an endogenous gene sequence (“knock out”). Any technique know in the art may be used to introduce an MURF1 or MAFBXtransgene into an animal to produce a founder line of transgenic animals, including pronuclear injection (Hoppe and Wagner, 1989, U.S. Pat. No. 4,873,191); retroviral mediated gene transfer into germ lines (Van der Puttenn, et al., 1985, Proc. Natl. Acad. Sci., USA 82, 6148-6152); gene targeting in embryonic stem cells (Thompson, et al., 1989, Cell 56, 313-321); electroporation or embryos (Lo, 1983, Mol. Cell Biol. 3, 1803-1814); and sperm mediated gene transfer (Lavitrano et al., 1989, Cell 57, 717-723). In addition, any technique may be used to produce transgenic animal clones containing a MURF1, MURF3 or MAFBXtransgene, for example nuclear transfer into enucleated oocytes of nuclei from cultured embryonic, fetal or adult cells induced to quiescence (Campbell, et al, 1996, Nature 380, 64-66; Wilmut, et al., Nature 385, 810-813). The invention provides for animals that carry the transgene in all of their cells as well as only some of their cells, for example, a particular cell type.

Before the present nucleic acids, polypeptides and methods for making and using the invention are described, it is to be understood that the invention is not to be limited only to the particular molecules or methods described. The molecules and method may vary, and the terminology used herein is for the purpose of describing particular embodiments. The terminology and definitions are not intended to be limiting since the scope of protection will ultimately depend upon the claims.

### EXAMPLES

### Example 1

Animal Model for Atrophy.

Skeletal muscle adapts to decreases in activity and load by undergoing atrophy, a process which involves a loss of total muscle mass and a consequent decrease in the size of individual muscle fibers. R. T. Jagoe, A. L. Goldberg, Curr. Opin. Clin. Nutr. Metab. Care 4, 183 (2001). Muscle atrophy occurs as a consequence of denervation, injury, joint immobilization, unweighting or bed-rest, glucocorticoid treatment, inflammatory diseases such as sepsis, cancer and old age (C. Rommel et al., Nature Cell Biology 3, 1009 (2001).).

To test for muscle atrophy, the ankle joint of rodents (mice or rats) are immobilized at 90 degrees of flexion. This procedure induces atrophy of the muscles with action at the ankle joint (e.g. soleus, medial and lateral gastronemius, tibilias anterior) to varying degrees. A reproducible amount of atrophy can be measured in hindlimb muscles over a 14-day period.

The immobilization procedure may involve either casting (mice) or pinning the ankle joint (rats). Rodents are anesthetized with ketamine/xylazine and the right ankle joint is immobilized. In rats, a 0.5 cm incision is made along the axis of the foot, over the heel region. A threaded screw (1.2×8 mm) is then inserted through the calcareous and talis, into the shaft of the tibia. The wound is closed with skin glue. In mice, the ankle joint is fixed at 90 degrees with a light weight casting material (VET-LITE) around the joint. The material is soaked in water and then wrapped around the limb. When the material dries it is hard, but light in weight.

At seven and 14 days following the immobilization, animals are anesthetized and killed by cervical dislocation. The tibialis anterior (TA), medial gastrocnemius (MG), and soleus (Sol) muscles are removed from the right (immobilized) and left (intact) hindlimbs, weighed, and frozen at a fixed length in liquid nitrogen cooled isopentane. A cohort of control animals which are the same weight and age as the experimental animals are also killed and the muscles removed, weighed and frozen. The amount of atrophy is assessed by comparing the weight of the muscles from the immobilized limb with the weight of the muscles from the control animals. Further assessment of atrophy will be done by measuring muscle fiber size and muscle tension output.

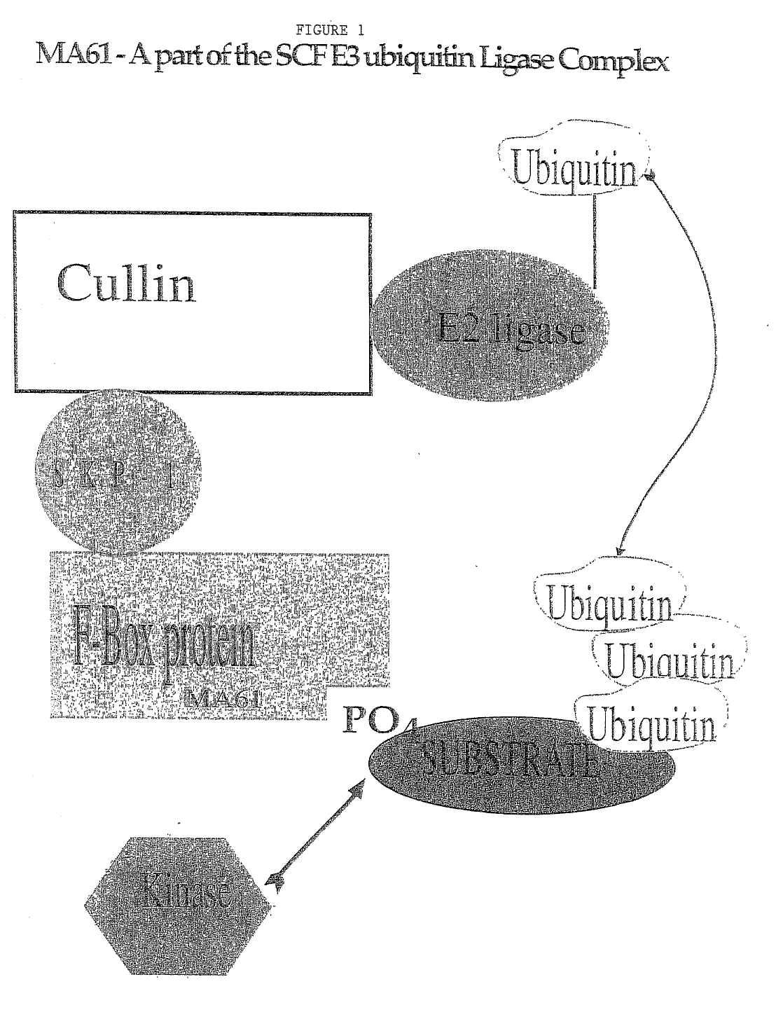

Denervation, immobilization (by joint fixation), and unweighting (by suspending the hindlimbs) in rats all result in similar rates of loss in mass of the medial gastrocnemius muscle (FIG. 1A), a result which is at least consistent with the idea that there are common mechanisms leading to atrophy. To determine if universal markers of atrophy exist, we initially compared gene expression in immobilization and denervation with a set of muscle-specific genes selected from the literature as changing during atrophy. Again, we saw surprising similarity in gene expression patterns between these two models (FIG. 1B, compare panel on left to center panel). However, when an unweighting model (hind-limb suspension) was analyzed none of the selected genes was similarly regulated to immobilization or denervation, indicating that these genes are not “universal” markers for the atrophy process (FIG. 1B). To identify potential universal markers of atrophy, we first attempted to identify genes regulated in one particular model (immobilization), and then determined which of these, if any, were similarly regulated in multiple other models (FIG. 1C).

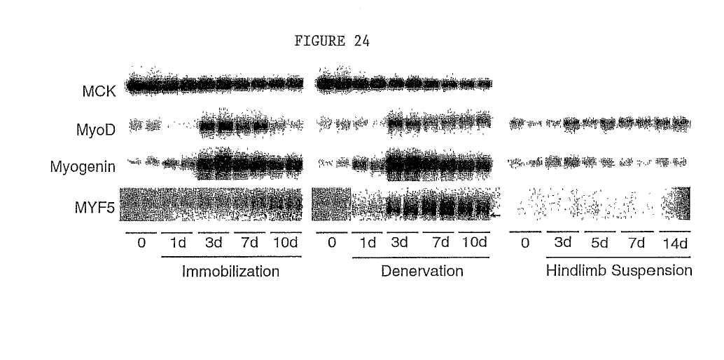

We performed Northern blots with RNA from the muscle of rats involved in three atrophy models: immobilization, denervation, and hindlimb-suspension. The Northern blots show the effect of atrophy on expression of muscle creatine kinase (MCK), myoD, myogenin and Myf5. Muscle was obtained from rats undergoing a time course (0, 1, 3, 7, and either 10 or 14 days, as indicated). For each lane, total RNA was pooled from three rat medial gastrocnemius muscles (MG). (FIG. 24).

We also performed an immunoblot of MuRF1 which demonstrates that MuRF1 protein is upregulated after ankle joint immobilization-induced atrophy (Imm). In FIG. 25A, Lane 1 is a control of recombinant rat MuRF1 (Accession number AY059627) expressed in COS cells. A lysate was made from these cells, so that the expected size of MuRF1 could be established. For lanes 2-7, protein lysates were pooled from three gastrocnemius muscles, taken from untreated rats (CON), rats at day one (Imm1) and day three (Imm3) after immobilization. An immunoblot is shown using an antibody raised against full-length rat MuRF1. Mammalian expression vectors coding for GST, GST-MAFbx, or GST-MAFbxDFb (an F-box deletion of MAFbx amino acids 216-263) were transiently transfected into Cos7 cells and the cells lysed 48 hours later in cold phosphate-buffered saline containing 1% NP40, 1 mM EDTA, 1 mM PMSF, 10 mg/ml aprotinin, 10 mg/ml leupeptin, 1 mM sodium orthovanadate, 25 mM beta-glycerophosphate, 100 nM okadaic acid, 20 nM microcystin LR, and 5 mM N-ethylmaleimide. Thirty microliters of glutathione-agarose beads (Amersham Pharmacia) was added to the clarified lysates (500 mg) and rotated for 3 hr at 4° C. Beads were washed three times by centrifugation with lysis buffer, boiled in reducing SDS sample buffer, and subjected to SDS-PAGE/immunoblot analysis with anti-Skp1 (Transduction Labs) and anti-Cullin 1 (Zymed). Muscle lysates (1 mg) were immunoprecipitated and immunoblotted with antisera raised against GST-MuRF1 which had been preabsorbed with immobilized GST.

Northern probes for mouse myoD spanned bp 571-938 of coding sequence; mouse myogenin spanned bp 423-861 of coding sequence mouse Myf5 spanned 406-745 of coding sequence. Northern probes for rat MuRF1 were made by PCR, spanning bp 24-612 of coding sequence. For mouse MuRF2, the probe was made using the 5′ PCR oligo: GAACACAGGAGGAGAAACTGGAACATGTC and the 3′ PCR oligo: CCCGAAATGGCAGTATTTCTGCAG, spanning the fifth exon of mouse MuRF2. For mouse MuRF3, the probe spanned bp 867-1101 of coding sequence. For rat MAFbx, the probe was made by PCR, and spanned bp 21-563 of coding sequence. For human MAFbx, the probe spanned bp 205-585. The Northern of mRNA from the MAFbx +/+, +/−, and −/− mice was probed with coding sequence spanning bp 660-840. To control for the amount of total RNA loaded, the agarose gels were stained with ethidium bromide and photographed, to assess ribosomal RNA bands. The Southern confirming the loss of the MAFbx allele on the 5′ end was performed with a mouse MAFbx genomic probe, spanning a 1.1 kb SacII fragment upstream of the ATG, and downstream of the indicated EcoRI site. The Northern of mRNA from the MuRF1 +/+, +/−, and −/− mice was probed with coding sequence spanning bp 1-500 of rat MuRF1 (accession AY059627). The Southern confirming the loss of the MuRF1 allele on the 5′ end was performed with a mouse MuRF1 genomic probe, spanning a 0.5 kb BglII fragment upstream of the ATG, and downstream of the indicated EcoRI site.

### Example 2

Cloning of the Rat MURF1 Gene, a Muscle-Specific Ring-Domain Gene

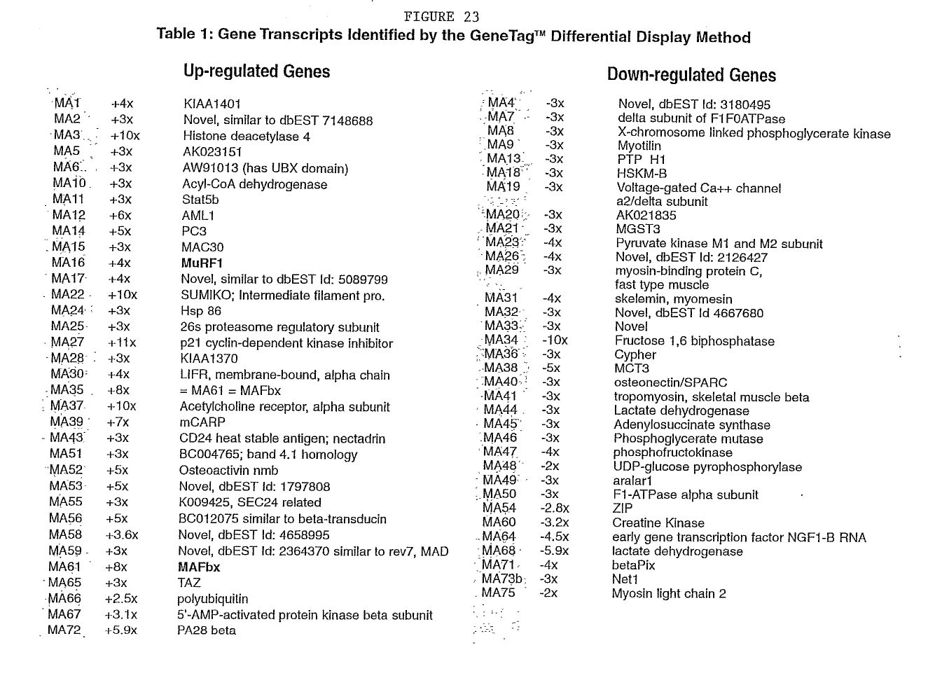

This experiment was performed in the interest of determining which genes are differentially expressed during conditions of skeletal muscle atrophy. The differential display analysis resulted in 74 transcripts, which were labeled MA1-MA74 (“MA” for Muscle Atrophy). Bioinformatic analysis on the original transcripts and on subsequent RACEd cDNA allowed for determinations in 61 of the transcripts. Transcript analysis was performed using the Genetag™ method (L. Y. Wong et al., Biotechniques 28, 776 (2000).) (FIG. 23)

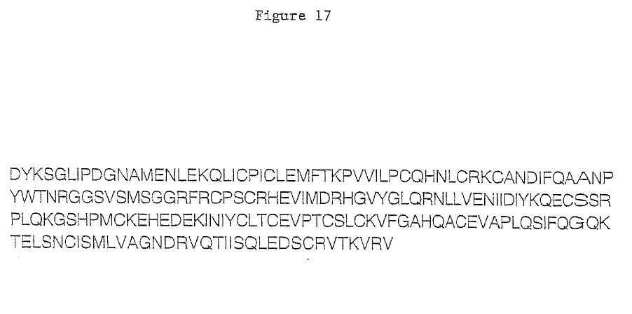

Rats were subjected to an atrophy-inducing model, as outlined in Example 1 supra. Three days after surgery, muscle tissue was harvested from the surgically treated animals. As a control, muscle tissue was also harvested from untreated animals. Messenger RNA was isolated from the atrophied muscle tissue and from the control muscle tissue, and put into a differential display assay. One of the gene transcripts found to be up-regulated during atrophy encompassed a 3′, untranslated part of the MURF1 transcript. This 3′ fragment was used to produce a DNA probe, which was used to clone a full-length gene comprising the coding sequence of MURF1. Also identified was an smaller, alternate splice form termed the rMURF1 VRV splice form. This alternate form differ from the full length form at the 3′ end, with the full length form being 152 amino acids longer. The alternate splice form has at its carboxy terminus the amino acid sequence “VRV” which is a PDZ-interacting domain (Torres R, Firestein B L, Dong H, Staudinger J, Olson E N, Huganir R L, Bredt D S, Gale N W, Yancopoulos G D (1998) Neuron:1453-63). The presence of a PDZ-interacting domain predicts that the protein is able to participate in protein-protein interactions. In contrast, the full length form has other protein interacting domains, for example, an acidic domain containing the amino acid sequence “DEEEEFTEEEEEEDQEE”. the presence of this domain predicts that this form is also able to interact with other proteins. The nucleotide and deduced amino acid sequences for full length rMURF1 are appended below in FIG. 6 and FIG. 7, respectively. The nucleotide and deduced amino acid sequences for the rMURF1 VRV splice form are appended below in Figure and FIG. 17 respectively.

### Example 3

Cloning of the Human MURF3 Gene, a Muscle-Specific Ring-Domain Gene

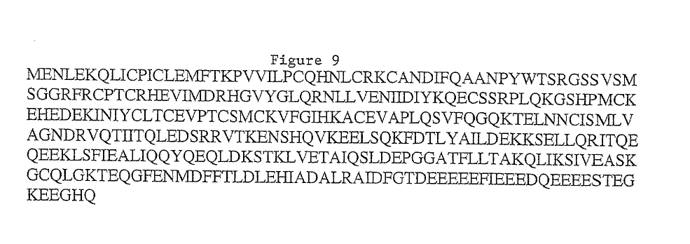

The rat MURF1 coding sequence was used to isolate human MURF3, by standard molecular biology techniques. This coding sequence has been previously deposited with American Type Culture Collection (ATCC®), as Human MA16 C8 in Stratagene T3/T7 vector, Patent Deposit Designation #PTA-1049, on Dec. 10, 1999. The nucleotide and deduced amino acid sequences for hMURF3 are appended below in FIGS. 8A-8C and FIG. 9, respectively. Human MuRF 1 was used to hybridize to rat MURF1, by standard techniques.

### Example 4

Cloning of Rat MA-61, a Muscle-Specific F-Box Gene

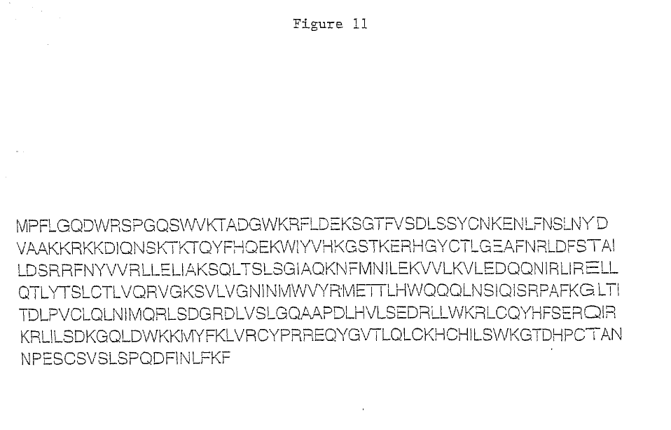

This experiment was performed in the interest of determining which genes are differentially expressed during conditions of skeletal muscle atrophy. To find such genes, rats were subjected to an atrophy-inducing model, as outlined in Example 1 supra. Three days after surgery, muscle tissue was harvested from the surgically treated animals. As a control, muscle tissue was also harvested from untreated animals. Messenger RNA was isolated from the atrophied and from the control muscle tissue, and put into a differential display assay. One of the gene transcripts found to be up-regulated during atrophy encompassed a 3′, untranslated part of the MAFBXtranscript. This 3′ fragment was used to produce a DNA probe, which was used to clone a full-length gene comprising the coding sequence of MA-61, by standard molecular biology techniques. The nucleotide and deduced amino acid sequences for rMAFBXare appended below in FIG. 10 and FIG. 11, respectively.

### Example 5

Cloning of the Human MAFBXgene, a Muscle-Specific F-Box Gene

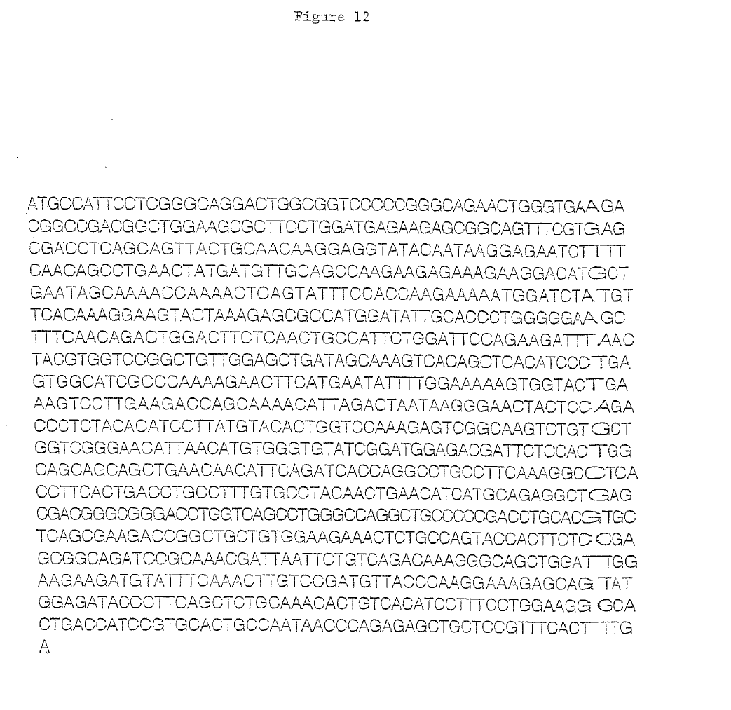

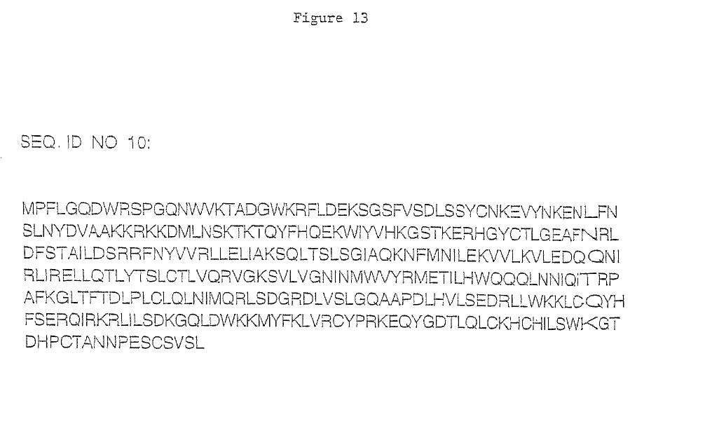

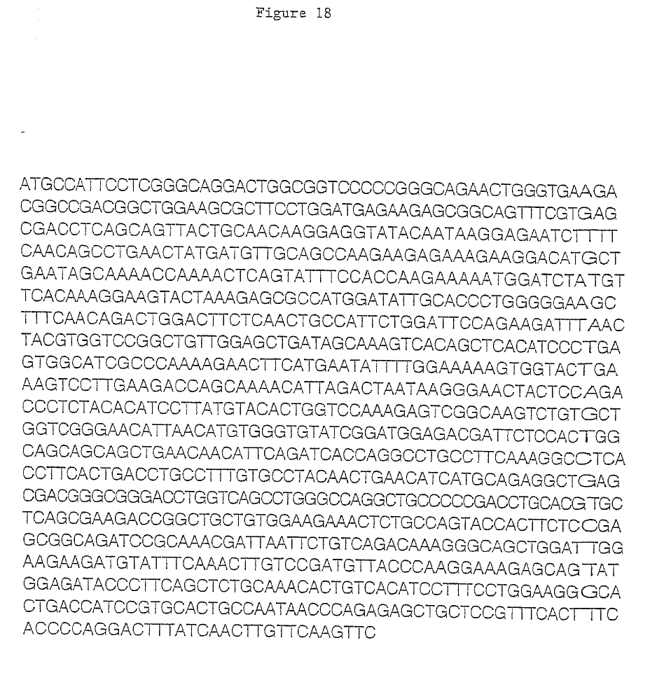

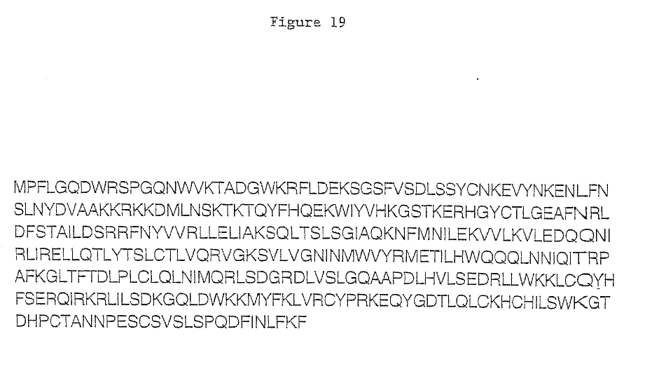

The rat MAFBXcoding sequence was used to isolate the human homolog of MAFBXD18, by standard molecular biology techniques. Two alternate forms of this gene were identified, termed hMAFBXD18 and hMAFBXK8. The D18 form of the gene encodes a protein which is 11 amino acids longer at the carboxy terminus than the K8 form. The significance of having two forms of this gene is unknown. However, it is often the case that alternate splice forms serve to modulate protein-protein interactions. These coding sequence has been previously deposited with American Type Culture Collection (ATCC®) as Human MAFBXK8 in Stratagene T3/T7 vector, Patent Deposit Designation #PTA-1048 and Human MAFBXD18 in Stratagene T3/T7 vector, Patent Deposit Designation #PTA-1050. The nucleotide and deduced amino acid sequences for hMAFBXK8 are appended below in FIG. 12 and FIG. 13, respectively. The nucleotide and deduced amino acid sequences for hMAFBXD18 are appended below in FIG. 18, and FIG. 19, respectively.

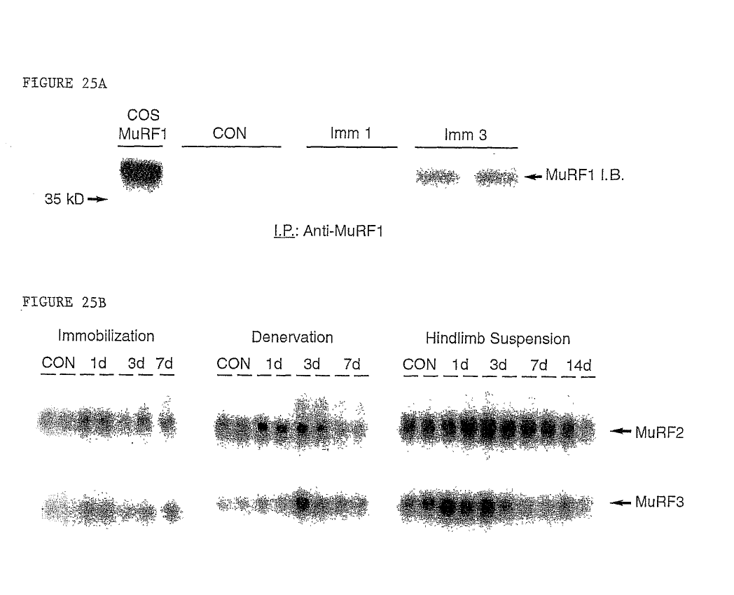

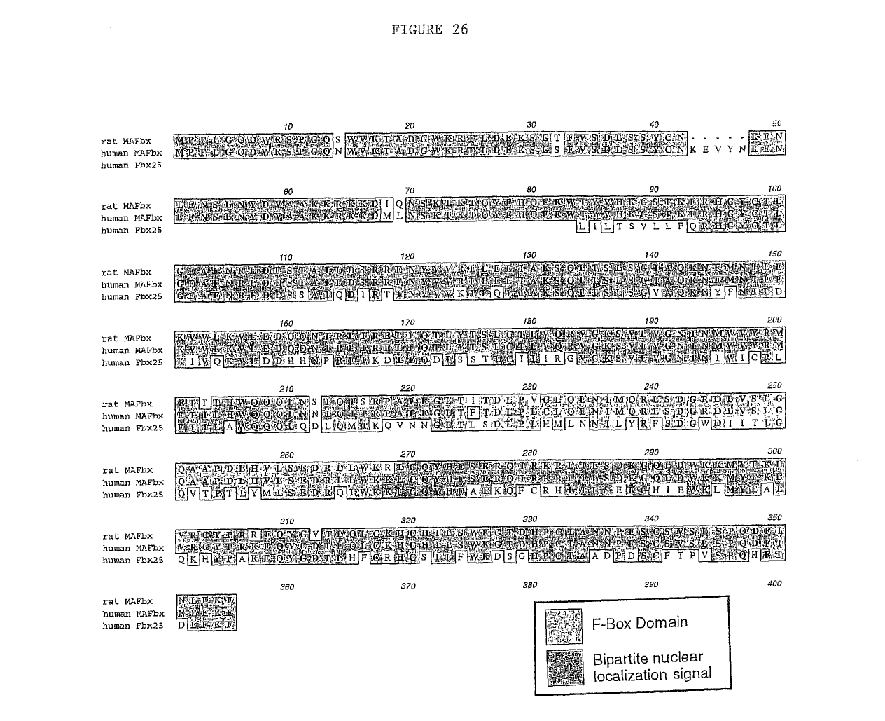

The sequences of rat and human MAFbx protein, and human Fbx25 were aligned (C. Cenciarelli et al., Curr. Biol. 9, 1177 (1999). The published partial Fbx25 sequence begins with the indicated Leucine (L) at amino acid 85 of MAFbx. The region surrounding the F-box is indicated, as is a bipartite nuclear localization signal. (FIG. 26) Accession numbers for rat and human MAFbx are AY059628 and AY059629, respectively.

### Example 6

Demonstration that MURF1 and MAFBXare Universal Markers for Muscle Atrophy.

After it was confirmed by Northern blot analysis that MURF1 and MAFBXare both up-regulated during immobilization-induced muscle atrophy, other models of muscle atrophy were examined. Muscle can undergo atrophy under a variety of stresses, including: denervation, in which the nerve to the muscle is severed; hind-limb suspension, in which the limb is physically suspended, to decrease muscle load; treatment with the glucocorticoid drug Dexamethasone. Northern analysis of mRNA obtained from muscle tissue subjected to each of these atrophying conditions demonstrated that MURF1 and MAFBXare up-regulated in every model of atrophy examined. Thus, MURF1 and MAFBXtranscriptional up-regulation can serve as clinical markers for muscle atrophy.

We first compared mRNA from rat skeletal muscle (medial gastrocnemius) which had been immobilized for three days to mRNA from control muscle, via the GeneTag™ differential display approach. We chose to analyze a relatively early time point (3 days), as opposed to a longer time point such as 14 days, in order to identify genes that may function as potential triggers, as well as markers, of the atrophy process. Only genes whose expression changed three-fold or higher were accepted as being differentially regulated. Acceptable transcripts were then assayed for “universality” by Northern analysis using panels of mRNA prepared from muscle subjected to denervation, immobilization or unweighting for periods of 1 to 14 days.

As a follow-up, mRNA from muscle which atrophied following systemic treatment with glucocorticoids or IL-1 was also analyzed. Finally, panels of mRNA prepared from muscle undergoing hypertrophy were examined to see if those genes regulated during atrophy were regulated in the opposite direction during hypertrophy.

One of the disadvantages of the differential display technique as performed was that the resultant cDNA obtained was often restricted to 3′ untranslated sequences, and of an average length of 75 base pairs. Thus it was often necessary to perform subsequent PCR-based 3′ and 5′ RACE analysis in order to obtain sufficient sequence to make gene identifications. The differential display analysis resulted in 74 transcripts, which were labeled MA1-MA74 (“MA” for Muscle Atrophy). Bioinformatic analysis on the original transcripts and on subsequent RACEd cDNA allowed for determinations in 61 of the transcripts (FIG. 23).

Several major classes of genes were regulated following joint immobilization-induced muscle atrophy. Genes involved in “energy-use pathways” constituted the largest class of down-regulated genes and included: lactate dehydrogenase, phosphofructokinase, and fructose 1,6 biphosphatase. Down-regulation of these pathways indicates that energy pathways can be regulated transcriptionally, as has been shown in the case of endurance exercise (K. Baar, E. Blough, B. Dineen, K. Esser, Exerc Sport Sci Rev 27, 333-379 (1999). The largest class of up-regulated genes were those associated with ubiquitylation and the proteasome pathway including: the 26s proteasome regulatory subunit p31, polyubiquitin, the proteasome activator subunit pa28 beta, and two novel ubiquitin ligases which will be discussed below. Although it has been previously shown that ATP-dependent protein degradation, via the addition of ubiquitin to target proteins and their subsequent proteolysis by the proteasome, is increased during muscle atrophy (R. Medina, S. S. Wing, A. Haas, A. L. Goldberg, Biomed Biochim Acta 50, 347-356 (1991); S. Temparis et al., Cancer Res 54, 5568-73 (1994); R. Medina, S. S. Wing, A. L. Goldberg, Biochem J 307, 631-637 (1995), it was not clear which if any of the genes involved in ubiquitylation might constitute markers for the atrophy process, or whether any of these genes were actually required, or even sufficient, to induce atrophy.

While the majority of genes perturbed during immobilization were similarly regulated during denervation, most of these genes were unaltered in the unweighting model (data not shown), despite the fact that similar rates of atrophy were seen in these models between one and seven days(FIG. 1A).

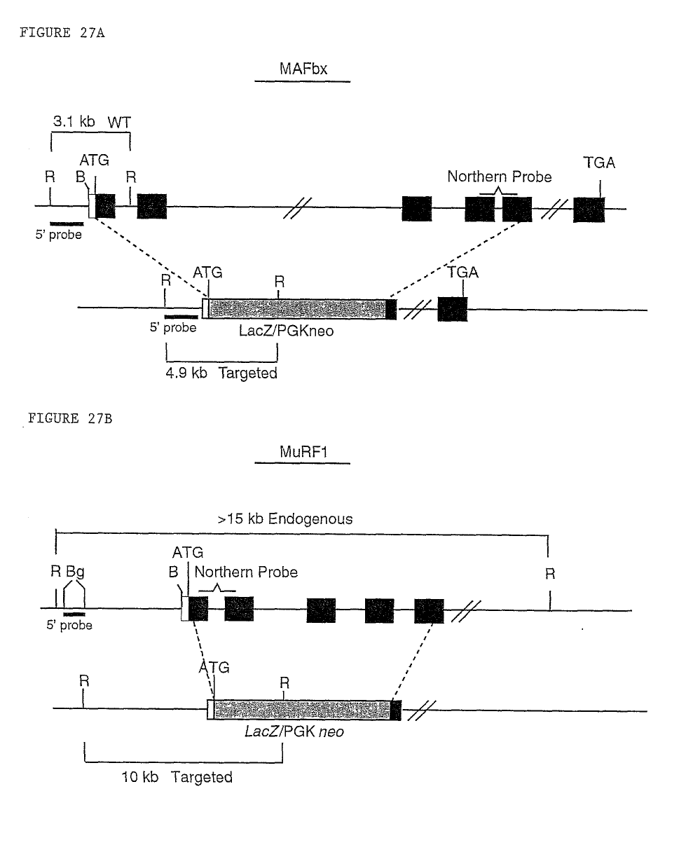

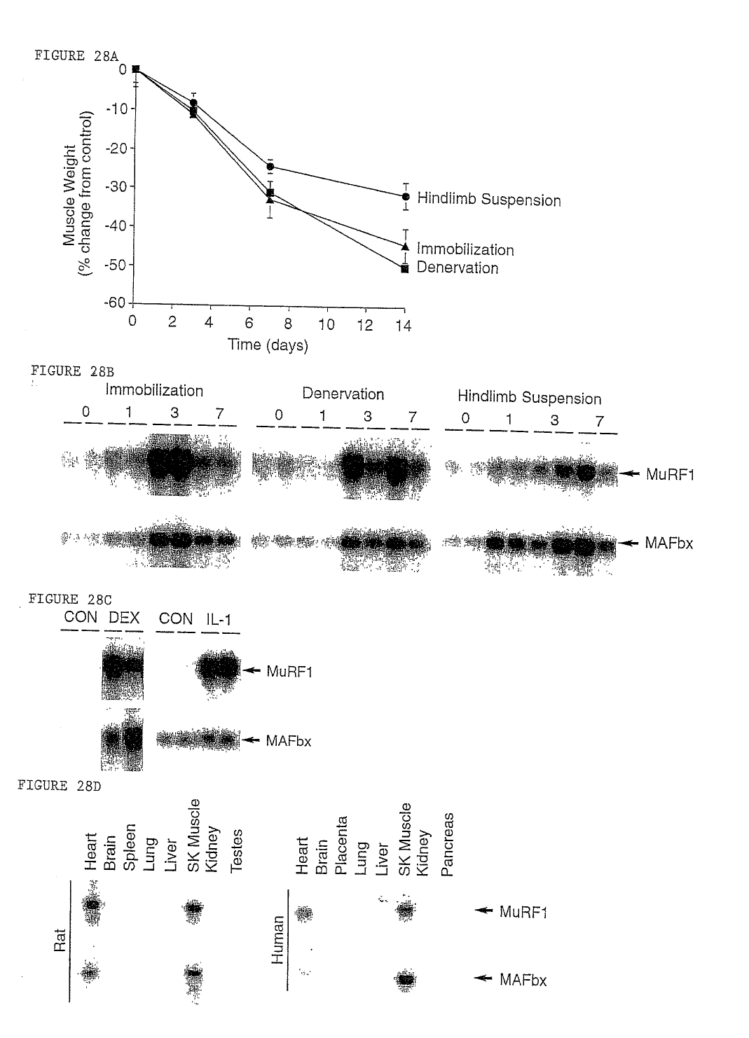

A time course of rat medial gastrocnemius muscle mass loss was examined in three in vivo models: Denervation, Immobilization and Hindlimb Suspension. Female Sprague Dawley rats weighing 250-275 gm were used in all models. For the denervation procedure: the right sciatic nerve was cut in the mid-thigh region, leading to denervation of the lower limb muscles. For the immobilization procedure: the right ankle joint was fixed at 90° of flexion by inserting a screw (1.2×8 mm) through the calcaneous and talis, into the shaft of the tibia. For the Hindlimb Suspension procedure: the hind limbs were unloaded by suspending the rats by their tails using a tail-traction bandage as described (D. B. Thomason, R. E. Herrick, D. Surdyka, K. M. Baldwin, J. Appl. Physiol. 63, 130 (1987). On the indicated days, rats were killed and hind limb muscles were removed, weighed and frozen. Weight-matched untreated rats served as controls. Data are means±s.e.m., n=10 rats. (FIGS. 28A-28DA).

Northern blots were also performed showing the effect of atrophy on MuRF1 and MAFbx transcripts. Medial gastrocnemius muscle was obtained from rats undergoing a time course (0, 1, 3, and 7 days) of three atrophy models: Ankle-Joint Immobilization, Denervation, and Hindlimb-Suspension. (FIGS. 28A-28D B)

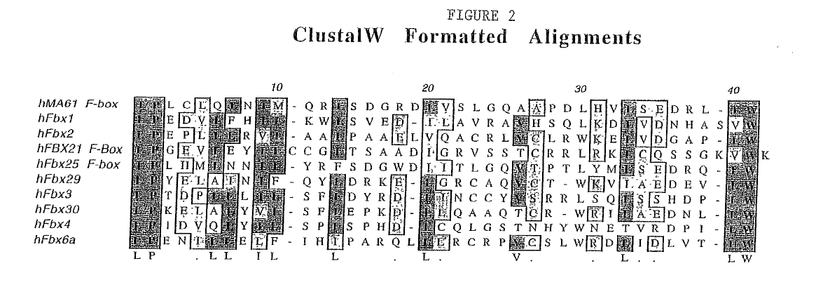

These findings indicate that denervation and immobilization are easily distinguishable transcriptionally from unweighting, perhaps because unweighting is unique in that there is relatively normal neural activation and joint movement in the suspended limbs. However, we did identify two genes that were up-regulated in all three models of atrophy; MA16, later identified as MuRF1 (for muscle-specific ring finger protein), and MA61, (subsequently called MAFbx, for Muscle Atrophy F-box protein) (FIG. 2A).

MuRF1 and MAFbx expression were analyzed in two additional models of skeletal muscle atrophy: treatment with the cachectic cytokine, interleukin-1 (IL-1) (R. N. Cooney, S. R. Kimball, T. C. Vary, Shock 7, 1-16 (1997)) and treatment with the glucocorticoid, dexamethasone(A. L. Goldberg, J Biol Chem 244, 3223-9 (1969).). While the first three models induced muscle atrophy by altering the neural activity and/or external load a muscle experiences to various degrees, these additional models induce atrophy without directly affecting those parameters. Northern blots were performed showing the effect of dexamethasone (DEX) and Interleukin-1 (IL-1) on expression of MuRF1 and MAFbx. Medial gastrocnemius muscle was obtained from untreated rats (CON), and from rats treated with DEX, delivered orally at a concentration of 6 μg/ml for nine days, and from rats treated with IL-1, delivered subcutaneously daily at a dose of 0.1 mg/kg for three days. FIGS. 28A-28D(c). Both cachectic agents caused an up-regulation of MuRF1 and MAFbx, with dexamethasone resulting in a greater than ten-fold increase in expression of MuRF1 and MAFbx (FIG. 2B).

Identification of a gene whose expression was up-regulated during atrophy and down-regulated during hypertrophy would greatly strengthen the claim that this gene was a marker for the atrophy phenotype, and provide correlative evidence that the gene of interest may function as a direct mediator of the atrophy process. We therefore examined MuRF1 and MAFbx expression in two models of skeletal muscle hypertrophy: hind-limb reloading following a 14-day unweighting period (D. B. Thomason, R. E. Herrick, D. Surdyka, K. M. Baldwin, J Appl Physiol 63, 130-7. (1987).), and compensatory hypertrophy in which the gastrocnemius and soleus muscles are removed, leaving the plantaris muscle to compensate for the loss of these synergistic muscles (G. R. Adams, F. Haddad, J Appl Physiol 81, 2509-16. (1996); R. R. Roy et al., J Appl Physiol 83, 280-90. (1997). In both of these models, MuRF1 and MAFbx expression decreased, demonstrating that these genes are not only positively correlated with atrophy, but are also negatively correlated with hypertrophy (FIG. 2C). Furthermore, Northern analysis on both rat and human “tissue blots” identified MuRF1 and MAFbx as being muscle-specific, in both heart and skeletal muscle (FIG. 2D), consistent with their serving specific roles in these tissues.

Total RNA obtained from rat and human tissues (Clontech) was hybridized with probes for the indicated genes. (FIGS. 28A-28DD)

### Example 7

Demonstration that MURF1 Can Function in a Ubiquitin Ligase Complex.

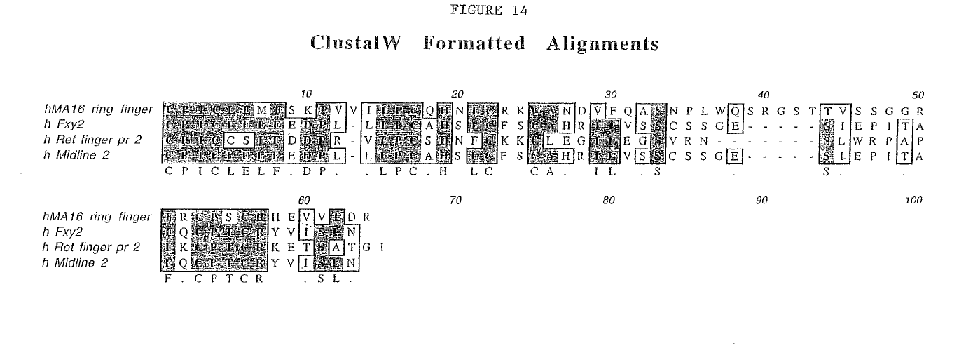

Recently, it has been shown that genes containing ring domains can function as “monomeric ubiquitin ligases”. Under certain conditions, these proteins simultaneously bind a substrate and a ubiquitin ligase, causing ubiquitination and proteosome-mediated degradation of the substrate. In the process, the ring domain protein itself becomes ubiquitinated. A vector encoding the rat MURF1 gene was transfected into COS cells, along with a vector encoding an HA-epitope-tagged form of ubiquitin. Protein lysates were harvested from the COS cells. MURF1 was immune-precipitated from the lysate using an antibody raised against an MURF1 peptide. The immune-precipitated protein was subjected to Western blot analysis, utilizing an antibody to the HA-tag. It was seen that MURF1 is highly ubiquitinated. Further, as a control, a vector encoding a mutant form of MURF1, in which the ring domain portion of the gene was deleted, was co-transfected into COS with tagged ubiquitin. In this case, no ubiquitination was evident. These results are consistent with the hypothesis that MURF1 functions as part of a ubiquitin complex, and that the ring-domain is necessary for ubiquitination, as seen in other ring domain proteins. FIG. 14 is a comparison of hMURF1 with other ring finger proteins.

MuRF1 was previously cloned by virtue of its interaction in a yeast two-hybrid experiment with a construct encoding a 30 kD domain of the large (300 kD) sarcomeric protein titin (T. Centner et al., J Mol Biol 306, 717-726 (2001)). While the presence of a “Ring finger domain (K. L. Borden, P. S. Freemont, Curr Opin Struct Biol 6, 396-401 (1996); P. S. Freemont, Ann NY Acad Sci 684, 74-192 (1993).)” in MuRF1 was previously noted, no further analysis was done to see if MuRF1 might function as a ubiquitin ligase. We noted that MuRF1 contains all the canonical structural features of ring-domain-containing monomeric ubiquitin ligases (P. S. Freemont, Curr Biol 10, R84-87 (2000); C. A. Joaeiro, A. M. Wiessman, Cell 102, 549-552 (2000).), and further reasoned that a ubiquitin ligase that could target muscle proteins for degradation would be a strong candidate for mediating muscle atrophy.

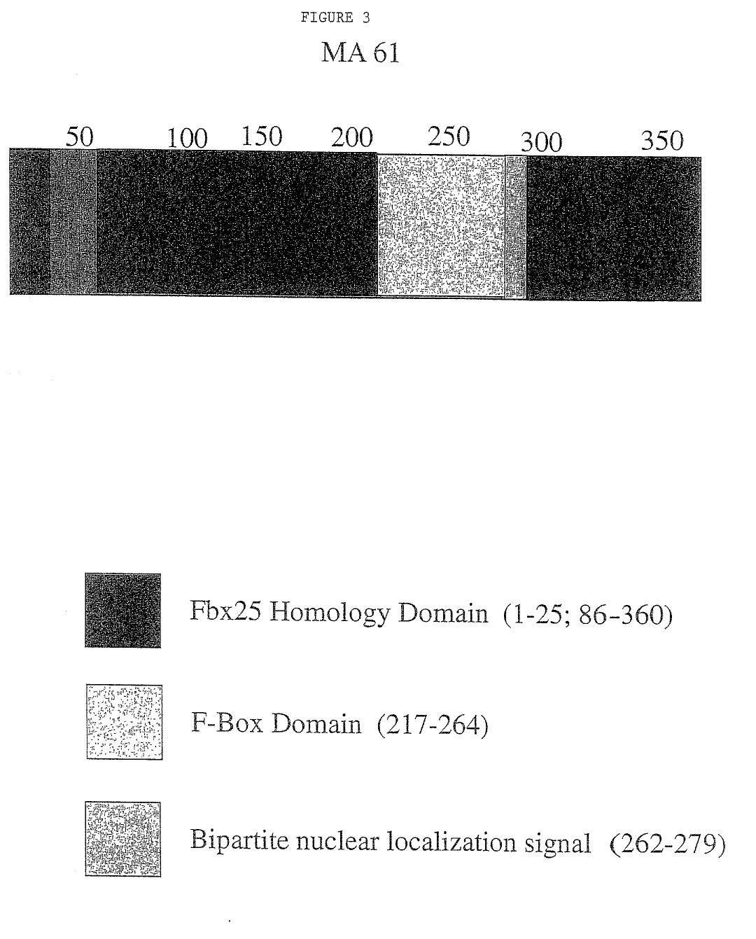

To initiate characterization of the MuRF1 protein and its potential ubiquitin ligase activity, we first demonstrated that MuRF1 protein levels, in addition to mRNA expression levels, increased during atrophy by immuno-blotting muscle lysates obtained from animals subjected to immobilization with an antibody which recognized MuRF1 (FIG. 3A). Next, recombinant MuRF1 protein was produced, and tested for ubiquitin ligase activity in an in vitro assay using radio-labeled ubiquitin as a substrate. MuRF1 was shown to be a potent ubiquitin ligase (FIG. 3B) in that no ubiquitin ligase activity was detected in the absence of MuRF1 (FIG. 3B) and other ring-finger ubiquitin ligases tested in this assay were less potent than MuRF1, as determined by the amount of radio-labeled ubiquitin self-conjugates per ug of protein.

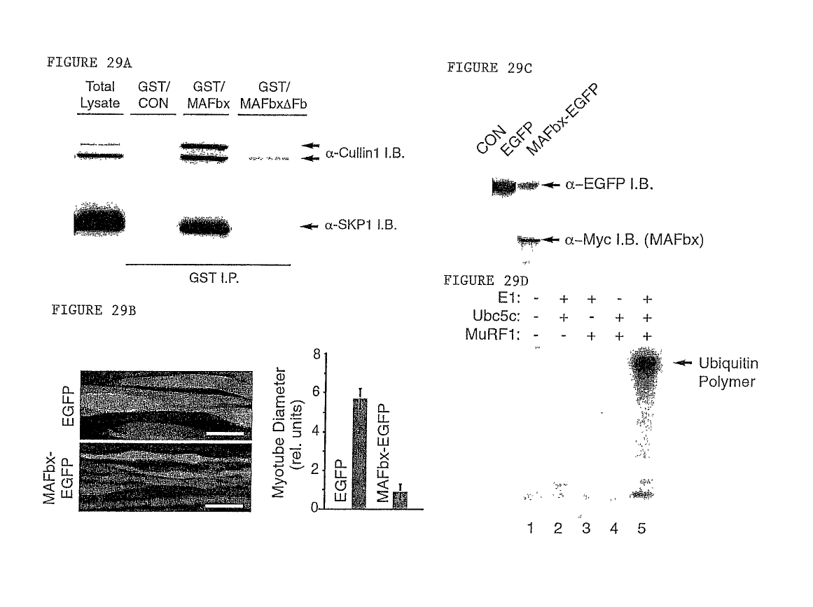

MuRF1 protein has ubiquitin ligase activity. Purified Glutathione-Sepharose-bound-MuRF1 protein (GST-MuRF1) was added to a ubiquitin ligase reaction as described (A. Chen et al., J. Biol. Chem. 275, 15432 (2000). Briefly, recombinant GST-MuRF1 (100 ng) was incubated with ³²P-ubiquitin (3 mg) in the presence of ATP, E1, and recombinant Ubc5c (FIGS. 29A-29D(D), lane 5). In lanes 1-4, indicated components were omitted. Aliquots of the reaction were analyzed by 12.5% SDS-PAGE to detect ³²P-labelled high molecular weight ubiquitin conjugates. The “ubiquitin polymer” may include ubiquitinated Ubc5c and MuRF1. FIGS. 29A-29DD.

### Example 8

Demonstration that MAFBXcan Function in an “SCF” Ubiquitin Ligase Complex.

Recently, it has been shown that genes containing F-box domains can function as part of a ubiquitin ligase complex called an “SCF” complex, where S stands for the gene product SKP1, C stands for a gene product called Cullin, and “F” stands for an F-box protein. To determine whether MAFBXis part of an SCF complex, MAFBXwas studied to determine if it binds to either SKP1 or Cullin, by doing a co-immune precipitation assay Vectors encoding GST (GST/CON), GST-MAFbx, or GST-MAFbxDFb (an F-box deletion of MAFbx, aa 216-263) were transiently transfected into Cos7 cells. Both Cullin1 and SKP1 could be co-purified, using glutathione-agarose beads, from lysates of cells transfected with GST-MAFbx (See FIGS. 29A-29D(A), Lane 3). Deletion of the F-box markedly reduced the amount of Cullin1 and Skp1 which co-precipitated (See FIGS. 29A-29D(A), Lane 4).

Over-expression of MAFbx causes atrophy. C2C12 myotubes, either uninfected (CON), or infected with an adenovirus expressing EGFP, or an adenovirus expressing both a Myc-epitope tagged rat MAFbx gene, and EGFP (MAFbx-EGFP). At day 4 after differentiation, fluorescent myotubes were photographed and myotube diameters were measured (right). The adenoviruses were generated as described (T.-C. He et al., Proc. Natl. Acad. Sci. USA 95, 2509 (1998).) . Calibration=50 mm. FIGS. 29A-29D(B)

Since the EGFP and MAFbx-EGFP viruses contained the EGFP gene, an anti-EGFP immunoblot (I.B.) allowed for a relative determination of infection levels. An immunoblot (I.B.) of lysates confirmed the presence of Myc-epitope tagged MAFbx protein in the myotubes infected with the MAFbx virus. FIGS. 29A-29DC.

These results are consistent with the hypothesis that MAFBXfunctions as part of an SCF ubiquitin ligase complex, and that the F-box-domain is necessary for association, as seen with other members of this complex.

### Example 9

Demonstration that a Substrate of MURF3 is the Syncoilin Gene.

To determine potential substrates for MURF3, a “yeast two-hybrid” experiment was performed. This is a standard method to detect proteins which co-associate with the protein of interest. In this experiment, a vector encoding the gene of interest is con-transfected, and fused to a yeast LexA domain, with a library encoding cDNA fused to GAL4-domain. If a cDNA in the library associates with the test gene, then the LexA and GAL4-domains are brought together, resulting in the production of a critical yeast protein, allowing the yeast to live in a particular medium. Using this method, we determined that a substrate for MURF3 is a recently-cloned gene called Syncoilin.

### Example 10

Clenbuterol Treatment, which Blocks Atrophy, Blocks Up-Regulation of MURF1 and MA-61.

To further establish whether MURF1 and MAFBXmay be markers for the muscle atrophy process, and potential targets to block atrophy, a drug called Clenbuterol was used to inhibit muscle atrophy, to see if this inhibition correlated with a decrease in the up-regulation of MURF1 and MA-61. Clenbuterol, a beta-adrenergic agonist, has been established as an inhibitor of muscle atrophy (see for example: Sneddon A A, Delday M I, Maltin C A, (2000). Amelioration of denervation-induced atrophy by clenbuterol is associated with increased PKC-alpha activity (Am J Physiol Endocrinol Metab 2000 July;279(1):E188-95).

Rat limb muscles were immobilized, as described in Example 1 supra. At the same time that the rats were immobilized, they were treated with Clenbuterol (3 mg/kg, s.c). Control immobilized animals were left untreated. Messenger RNA from control and clenbuterol-treated animals' muscle tissue was examined for MURF1 and MAFBXexpression by standard techniques (Northern hybridization using MURF1 and MAFBXprobes). It was found that treatment with clenbuterol, which significantly blocked atrophy, also blocked the up-regulation of MURF1 and MA-61.

### Example 11

Analysis of MuRF2 and MuRF3.

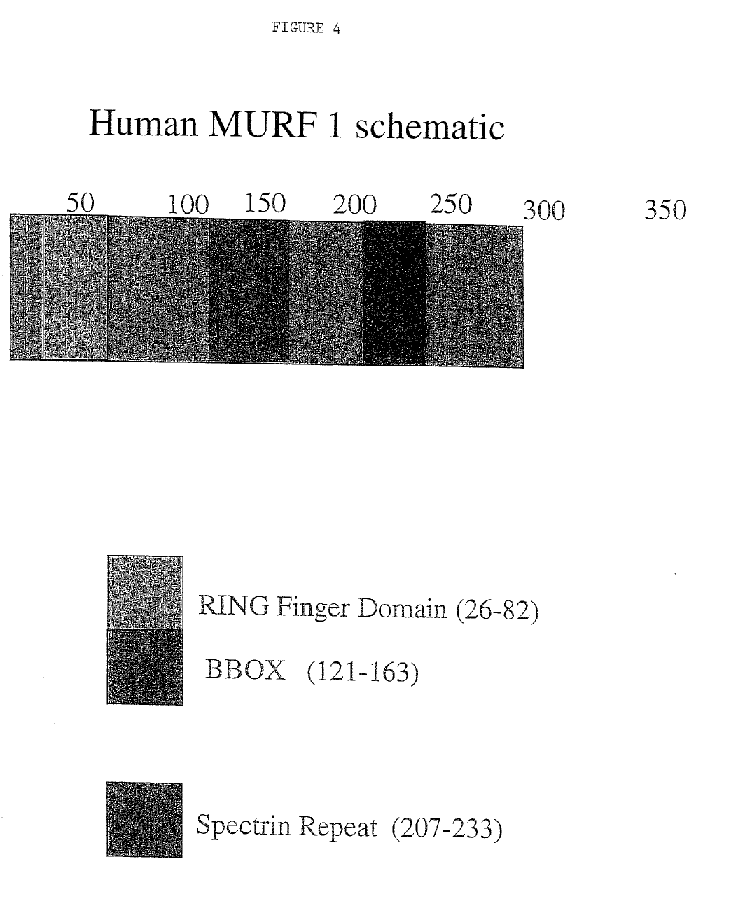

Two genes closely related to MuRF1 have been cloned, and named MuRF2 and MuRF3 ,(T. Centner et al., J Mol Biol 306, 717-726 (2001), J. A. Spencer, S. Eliazer, R. L. Ilaria, J. A. Richardson, E. N. Olsen, J. Cell Biol. 150, 771-784 (2000)). Northern analysis demonstrated that MuRF2 and MuRF3 expression were not consistently up-regulated during skeletal muscle atrophy (FIG. 4C), despite being muscle specific and highly homologous to MuRF1 (T. Centner et al., J Mol Biol 306, 717-726 (2001).). Muscle was obtained from rats undergoing a time course (0, 1, 3, and 7 days) of three atrophy models: immobilization, denervation, and hindlimb-suspension. For each lane, total RNA was pooled from three rat medial gastrocnemius muscles (MG). Northern hybridizations were performed with probes for the indicated genes. Northern probes for mouse myoD spanned bp 571-938 of coding sequence; mouse myogenin spanned bp 423-861 of coding sequence mouse Myf5 spanned 406-745 of coding sequence. Northern probes for rat MuRF1 were made by PCR, spanning bp 24-612 of coding sequence. For mouse MuRF2, the probe was made using the 5′ PCR oligo: GAACACAGGAGGAGAAACTGGAACATGTC and the 3′ PCR oligo: CCCGAAATGGCAGTATTTCTGCAG, spanning the fifth exon of mouse MuRF2. For mouse MuRF3, the probe spanned bp 867-1101 of coding sequence. To control for the amount of total RNA loaded, the agarose gels were stained with ethidium bromide and photographed, to assess ribosomal RNA bands. It is unknown whether MuRF2 or MuRF3 function as ubiquitin ligases.

### Example 12

Ubiquitination Increases During Muscle Atrophy.

As demonstrated supra, MURF1 is part of a ring domain ubiquitin ligase, and MAFBXis part of an “SCF” ubiquitin ligase complex. To show that ubiquitination is involved in the process of muscle atrophy, a Western blot was performed on protein obtained from control muscle tissue and from muscle tissue undergoing denervation or immobilization-induced atrophy. In both atrophy conditions, it was seen that the level of ubiquitination increases during atrophy. This point has also been established in the literature (see for example: Solomon V, Baracos V, Sarraf P, Goldberg A L. (1998)) Rates of ubiquitin conjugation increase with atrophy, largely through activation of the N-end rule pathway. (Proc Natl Acad Sci USA. 1998 October 13;95(21):12602-7).

### Example 13

MAFBXis a Member of the SCF E3 Ubiquitin Ligase Family, as Demonstrated by Yeast Two-Hybrid Association Between MAFBXand Skp1.

We cloned full-length rat and human cDNAs for this gene. Open reading frames of rat and human MAFbx cDNA sequence predict proteins which are 90% identical (FIG. 4A). The protein sequences are notable for the presence of an “F-box” domain, which is of interest since F-box domains have been identified in proteins which are members of a particular E3 ubiquitin ligase called an “SCF ubiquitin-ligase complex” (D. Skowyra, K. L. Craig, M. Tyers, S. J. Elledge, J. W. Harper, Cell 91, 209-19 (1997); J. Lisztwan et al., EMBO J 17, 368-83 (1998).). The SCF complex is thus named because it involves stable interactions between the following proteins: Skip1 (Skp1), Cullin1 (Cul1), and one of many “F-box”-containing proteins (Fbps). More than thirty-eight different Fbps have been identified in humans (J. T. Winston, D. M. Koepp, C. Zhu, S. J. Elledge, J. W. Harper, Curr Biol 9, 1180-2 (1999); C. Cenciarelli et al., Curr Biol 9, 1177-9 (1999)). The closest relative to MAFbx is Fbx25, a gene previously cloned in a large search for F-box containing proteins. Interestingly, whereas MAFbx expression is limited to skeletal muscle and heart, Fbx25 is expressed in most other tissues, but not in skeletal muscle (data not shown). We demonstrated that MAFbx is in fact an SCF-type E3 ubiquitin ligase in two ways. First, yeast-two hybrid cloning using full-length MAFbx as a “bait” resulted in 94 independent clones of Skp1, out of a total of 94 clones obtained in the interaction experiment (data not shown). Second, immune-precipitation of MAFbx from mammalian cells transfected with MAFbx resulted in the co-precipitation of both Skp1 and Cul1 (FIG. 4B). This co-precipitation was dependent on the presence of the F-box domain in MAFbx (FIG. 4B, compare lanes 3 and 4). The F-box motif has been shown to be necessary for interaction between Fbps and Skp1 (E. T. Kipreos, M. Pagano, Genome Biol. 1 (2000).)

### Example 14

MURF1 Functions as a Ubiquitin Ligase.

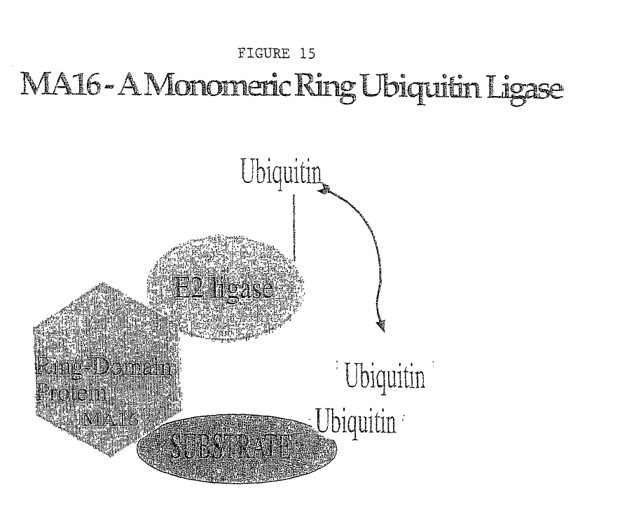

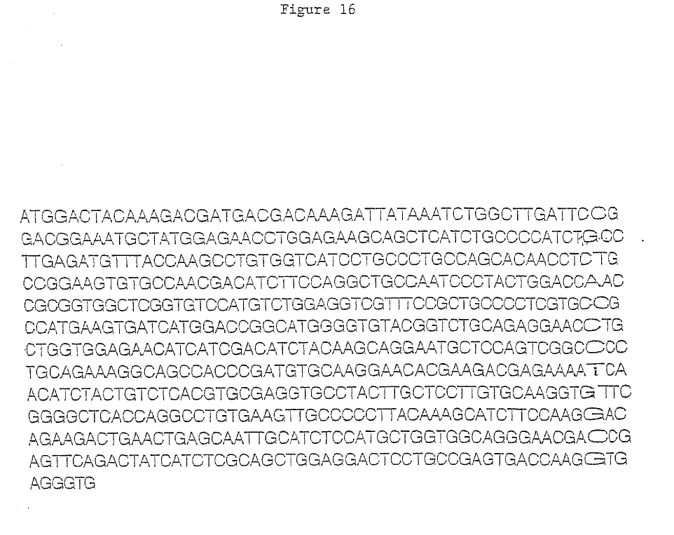

To determine whether MURF1 functions as a ubiquitin ligase, recombinant MURF1 protein was produced in E.Coli bacteria, using standard techniques. This recombinant protein was purified, and used in an in vitro ubiquitin ligase assay, as described in Chen et al., 2000, J Biol Chem, 275, pg 15432-15439. It was found that MURF1 was highly active; this activity is dependent on both E1 and UBC5c, as an E2 (E1 and E2 components are necessary for ring domain protein-mediated ubiquitin ligation). A negative control protein failed to work. Other ring domain-containing proteins, as positive controls, also functioned in the assay, but were less efficient, as measured by ubiquitin conjugation. See FIG. 15 for a schematic representation of how MURF1 functions as a ubiquitin ligase.

### Example 16

Knock-Out Animals

MAFBXknock-Out Animals Show a Decrease in Muscle Atrophy

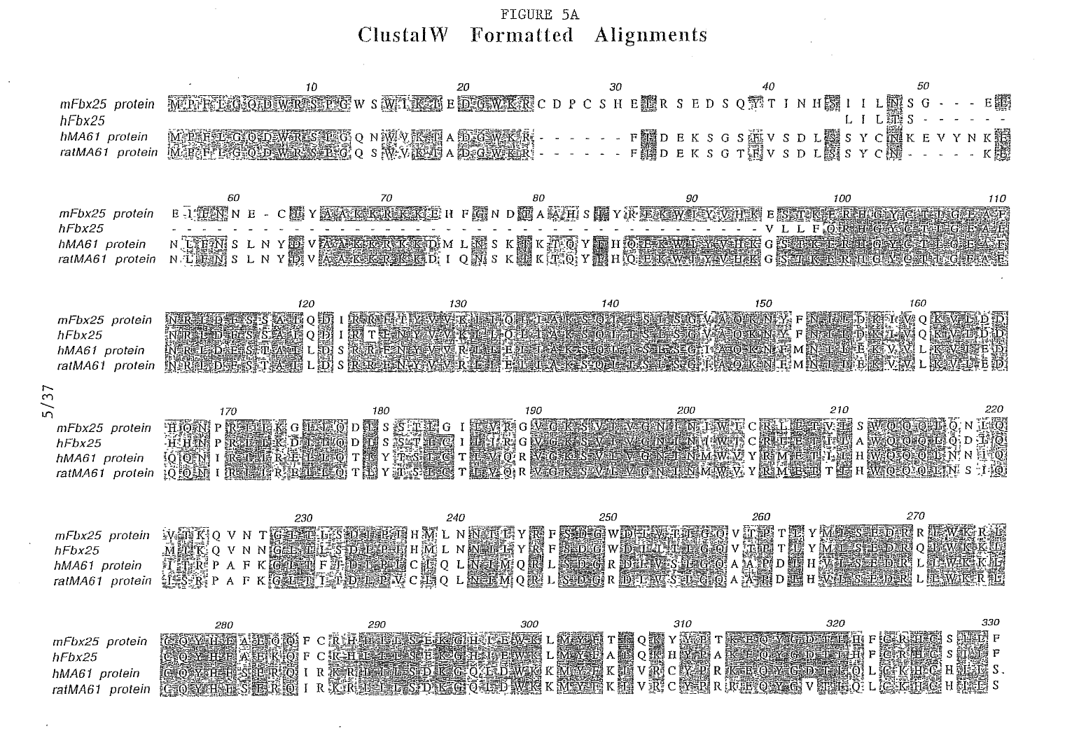

To further elucidate the function of MAFbx we genetically engineered a MAFbx null allele in mice, in which genomic DNA spanning the ATG through the exon encoding the F-box region was replaced by a LacZ/neomycin cassette, (FIG. 5A) allowing us to simultaneously disrupt MAFbx function and perform b-galactosidase (b-gal) staining to determine MAFbx expression patterns. Analysis of the MAFbx locus demonstrated the expected perturbation in MAFbx +/− and −/− animals (FIG. 5B). Further, MAFbx −/− animals were null for MAFbx mRNA (FIG. 5C). MAFbx −/− mice were viable, fertile and appeared normal. Mice deficient in MAFbx had normal growth curves relative to wild type litter mates, and skeletal muscles and heart had normal weights and morphology (data not shown).

Given the absence of an obvious phenotype, we decided to challenge the mice in an atrophy model to determine the role, if any, of MAFbx in producing skeletal muscle loss. Muscle atrophy was induced by cutting the sciatic nerve, resulting in denervation and disuse of the tibialis anterior and gastrocnemius muscles. Denervation resulted in up-regulation of the MAFbx gene locus in all muscle fibers, as demonstrated by b-gal staining in the tibialis anterior of MAFbx +/− mice (FIG. 6A). Significant muscle atrophy occurred in the tibialis anterior and gastrocnemius muscles of wild type, MAFbx +/+, mice at 7 and 14 days following denervation (FIG. 6B). Mice deficient in MAFbx (MAFbx −/−) had significantly less atrophy than MAFbx +/+ mice at both 7 and 14 days (FIG. 6B). In fact, MAFbx −/− mice exhibited no additional muscle loss between 7 and 14 days, whereas MAFbx +/+ continued to lose mass. The preservation of muscle mass at 14 days was also reflected in a preservation of mean fiber size and fiber size variability; MAFbx −/− mice had significantly larger fibers than the MAFbx +/+ mice, and maintained the same fiber size variability as seen in the undenervated limb (FIG. 6C). These data provide strong evidence that MAFbx is a required regulator of muscle atrophy, and that it may play an important role in the degradation of muscle proteins.

MuRF-1 Knock-Out Animals Show a Decrease in Muscle Atrophy

To further elucidate the function of MuRF1 we genetically engineered a MuRF1 null allele in mice, in which genomic DNA spanning the ATG through the exon encoding the F-box region was replaced by a LacZ/neomycin cassette, (FIG. 5A) allowing us to simultaneously disrupt MuRF1 function and perform b-galactosidase (b-gal) staining to determine MuRF1 expression patterns. Analysis of the MuRF1 locus demonstrated the expected perturbation in MuRF1 +/− and −/− animals (FIG. 5B). Further, MuRF1 −/− animals were null for MuRF1 mRNA (FIG. 5C). MuRF1 −/− mice were viable, fertile and appeared normal. Mice deficient in MuRF1 had normal growth curves relative to wild type litter mates, and skeletal muscles and heart had normal weights and morphology (data not shown).

In this study we identified two genes that are muscle-specific and up-regulated during muscle atrophy induced by a variety of perturbations. Both MuRF1 and MAFbx encode distinct types of E3 ubiquitin ligases. The discovery of two ubiquitin ligases as markers for multiple models of skeletal muscle atrophy suggests that highly disparate perturbations, ranging from denervation to glucocorticoid treatment, activate common atrophy-inducing pathways. Further, the particular function of ubiquitin ligases, to target discrete substrates for proteolyis by the ATP-dependent proteasome, suggests that a particular protein degradation pathway is up-regulated during atrophy and mediated by MAFbx and MuRF1.

MuRF1 contains a ring finger domain and was shown to function as a ubiquitin ligase in vitro, thereby suggesting that it may function in skeletal muscle as a monomeric ring-finger ligase. While this study did not identify a substrate, a previous study identified MuRF1 as binding to the sarcomeric protein titin, raising the possibility that MuRF1 might function as a ubiquitin ligase for titin, an important organizer of the sarcomeric complex (T. Centner et al., J Mol Biol 306, 717-726 (2001).).

MAFbx is a member of the F-box containing SCF family. No substrates have been determined for MAFbx in these studies; however, expression of MAFbx in skeletal myotubes in vitro was sufficient to induce atrophy in these cells. Further, mice deficient in MAFbx exhibited significantly less atrophy than wild-type mice in a denervation model. This finding demonstrates that MAFbx is a critical regulator of the muscle atrophy process, most likely through the regulation of the degradation of crucial muscle proteins. Analysis of these MAFbx deficient mice in additional atrophy and hypertrophy models will further elucidate the role of MAFbx in muscle atrophy and protein turnover.

Future studies will focus on the identification of substrates for MAFbx and MuRF1, and the further examination of mice lacking either MAFbx or MURF1, MuRF relatives, as well as various combinations. Preliminary analysis of mice deficient in MuRF1 show them to be viable, and normal in appearance and growth characteristics (data not shown). The current studies identify MuRF1 and MAFbx as markers of skeletal muscle atrophy, and potential targets for therapeutic intervention to prevent the loss of skeletal muscle in clinical settings of atrophy. Since both MuRF1 and MAFbx are also specifically expressed in heart muscle, it will also be important to examine the roles of these ubiquitin ligases in heart remodeling and disease.

Targeting of the MAFbx and MuRF1 Loci.

Targeting of the MAFbx locus. To generate a gene targeting vector for homologous recombination in murine ES cells, a BAC genomic clone was obtained by screening a Genome Systems 129 Sv/J genomic library, using a probe specific for the first coding exon of the MAFbx gene. The BAC contained a genomic DNA insert of approximately 95 kb and encompassed the entire MAFbx gene—which is comprised of 9 coding exons (as in the rat and human orthologs). To disrupt the MAFbx gene, a LacZ/neomycin cassette was inserted precisely at the ATG initiation codon, to allow for LacZ gene expression to be driven by the MAFbx promoter. The insertion of LacZ simultaneously replaced approximately 35 kb of MAFbx genomic sequences, containing coding exons 1-7 and most of exon 8. The F-box is encoded by exons 7 and 8 in the mouse, rat and human MAFbx genes. The targeting vector was linearized by digestion with Not1 and electroporated into CJ7 ES cells (T. M. DeChiara et al., Cell 85, 501 (1996). ES cell clones that survived selection in G418 were screened to identify homologously recombined heterozygous ES cells. Three targeted clones were identified from 65 clones screened yielding a recombination frequency of 4.6%. Se FIGS. 27A-27BA.

Targeting of the MuRF1 locus. To generate a gene targeting vector for homologous recombination in murine ES cells, a BAC genomic clone was obtained by screening a Genome Systems 129 Sv/J genomic library, using a probe specific for the first coding exon of the MuRF1 gene. The BAC contained a genomic DNA insert of approximately 33 kb and included the first five exons of the MuRF1 gene. To disrupt the MuRF1 gene, a LacZ/neomycin cassette was inserted precisely at the ATG initiation codon, to allow for LacZ gene expression to be driven by the MuRF1 promoter. The insertion of LacZ simultaneously replaced approximately 8 kb of MuRF1 genomic sequences, containing coding exons 1-4 and most of exon 5. The RING finger is encoded by exons 1 and 2 in the mouse, rat and human MuRF1 genes. The targeting vector was linearized by digestion with Not1 and electroporated into CJ7 ES cells ((T. M. DeChiara et al., Cell 85, 501 (1996). ES cell clones that survived selection in G418 were screened to identify homologously recombined heterozygous ES cells. Three targeted clones were identified from 22 clones screened yielding a recombination frequency of 14%. See FIGS. 27A-27BB.

Confirmation of Absence of Targeted Allele: MAFbx

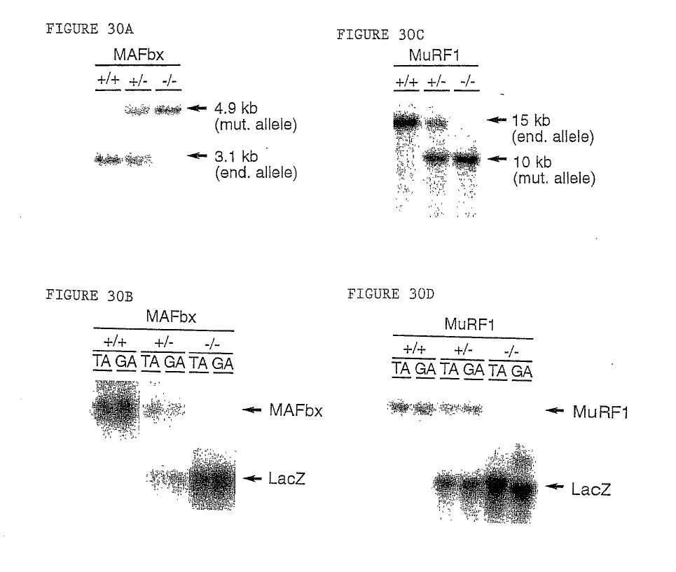

The targeting of the MAFbx gene was confirmed in ES cells, and in both heterozygous and homozygous MAFbx mutant mice, by digesting genomic tail DNA with EcoR1 and probing with a 5′ 1.1 kb SacII fragment to detect the endogenous (end. allele) 3.1 kb and targeted (mut. allele) 4.9 kb EcoR1 fragments. (FIGS. 30A-30DA).

The targeted mutation in the MAFbx gene was verified by probing mRNA from both tibialis anterior (TA) and gastrocnemius muscle (GA) prepared from MAFbx +/+, +/− and −/− mice with a MAFbx probe, spanning bp 660-840 of coding sequence (MAFbx; upper panel), as well as with a probe of the inserted LacZ gene (FIGS. 30A-30DB).

Confirmation of Absence of Targeted Allele: MuRF1

The targeting of the MuRF1 gene was confirmed in ES cells, and in both heterozygous and homozygous MuRF1 mutant mice, by digesting genomic tail DNA with EcoRI, and probing with a 5′ 0.5 kb BglII fragment to detect the endogenous (end. allele) 15 kb and targeted (mut. allele) 10 kb EcoR1 fragments. (FIGS. 30A-30D(C).

The targeted mutation in the MuRF1 gene was verified by probing mRNA from both tibialis anterior muscle (TA) and gastrocnemius muscle (GA) prepared from MuRF1 +/+, +/− and −/− mice with a probe spanning bp 1-500 of rat MuRF1 coding sequence (MuRF1, upper panel), as well as with a probe of the inserted LacZ gene (FIGS. 30A-30DD)

Confirmation that the MAFbx and MuRF1 Genes are Upregulated in Muscle Following Denervation.

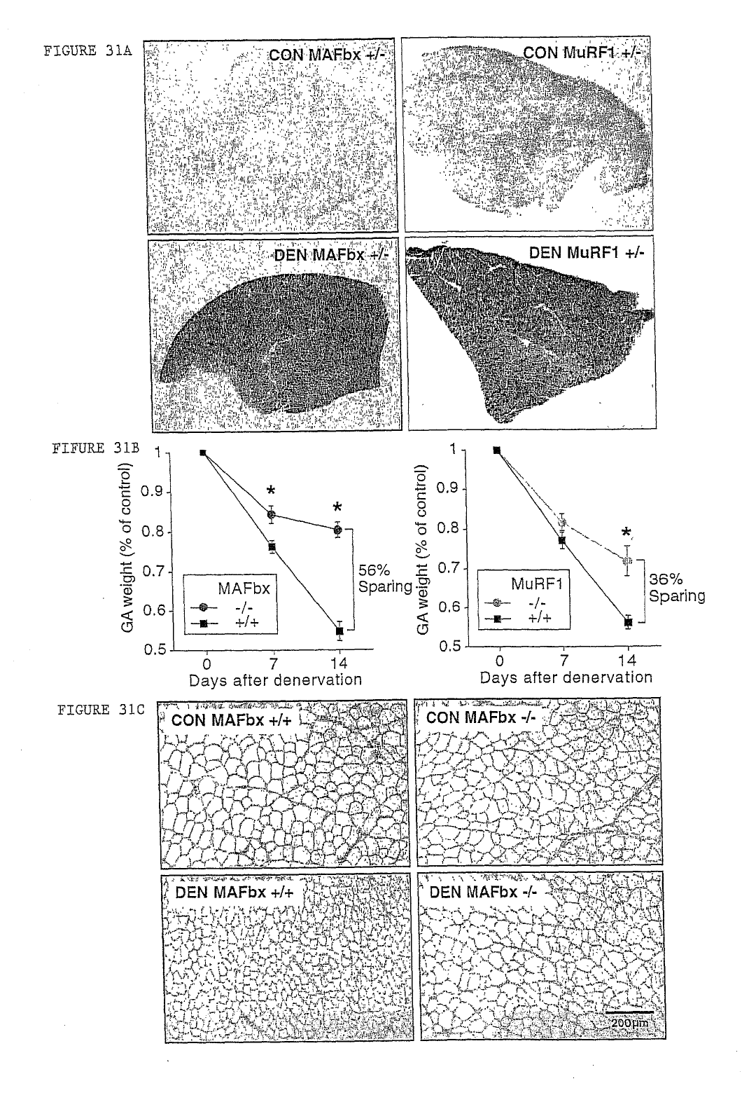

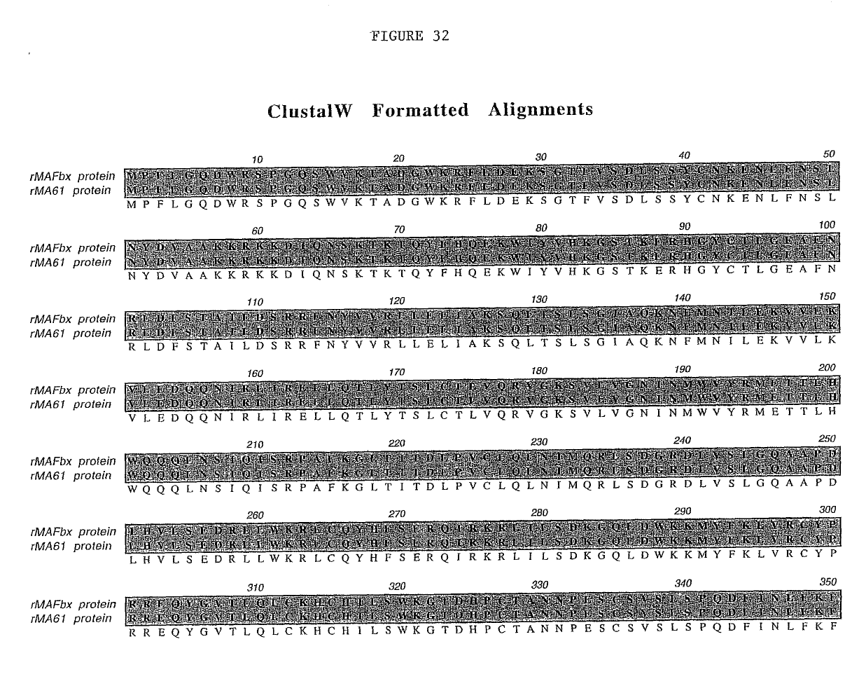

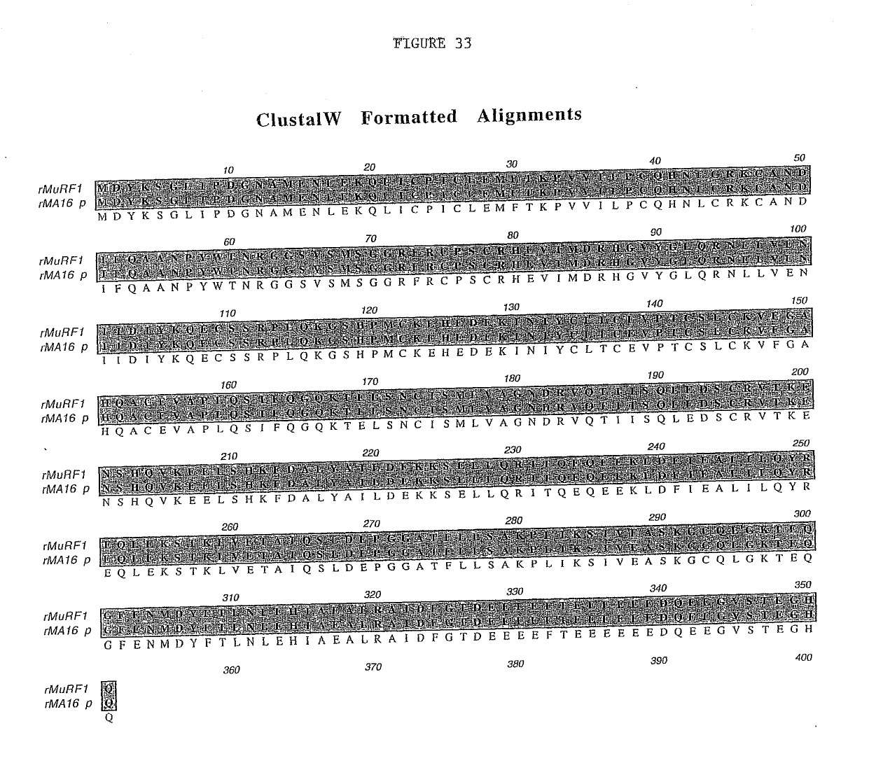

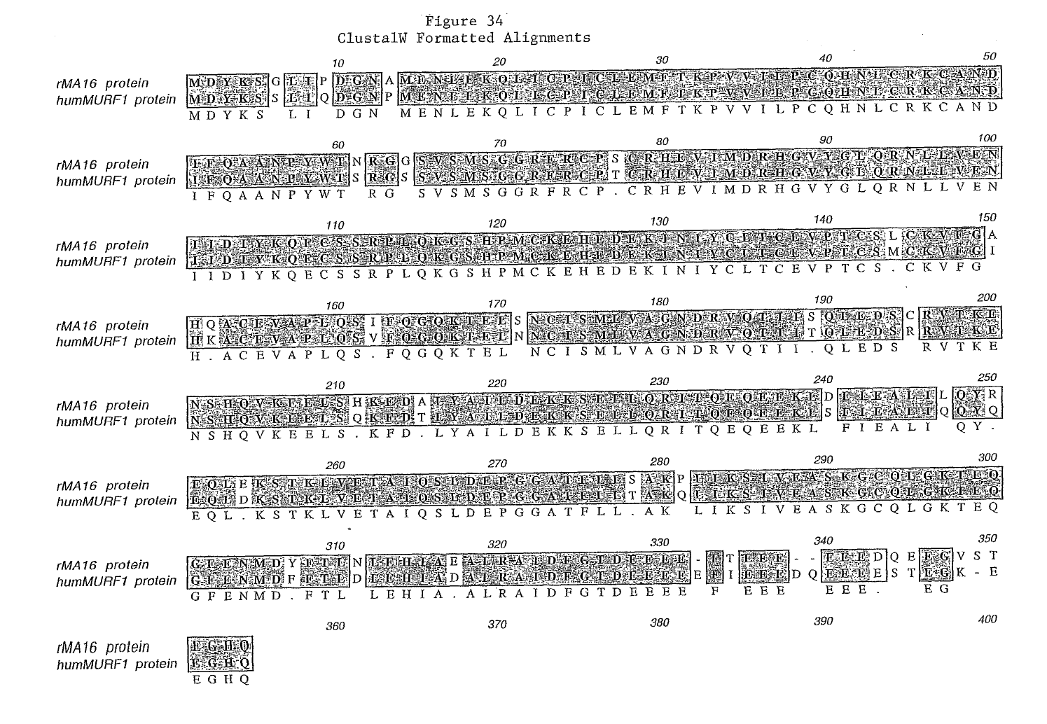

The regulation of the MAFbx and MuRF1 genes were examined using b-gal staining in MAFbx +/− and MuRF1 +/− mice. The right sciatic nerve was cut in heterozygous mice, resulting in denervation of the tibialis anterior (TA) muscle. Seven days later, the right and left tibialis anterior muscles were sectioned and stained for b-gal activity, in the same media, for equivalent times. In control muscle, there is a low level of MAFbx expression in some (primarily deep region), but not all, muscle fibers of the TA. In comparison, MuRF1 is expressed in all fibers at a slightly higher level than MAFbx. After denervation, both MAFbx and MuRF1 expression are upregulated in all muscle fibers. FIGS. 31A-31C(A).

Muscle mass from MAFbx and MuRF1 deficient was compared to wild type (+/+) mice, and it was found that the mice maintain muscle mass after denervation, as compared to wild type (+/+) mice. The right hindlimb muscles of adult mice (MAFbx +/+ and −/−) were denervated by cutting the right sciatic nerve. The left hindlimb of each animal served as its own control. At 7 and 14 days following denervation, the right and left gastrocnemius muscle complex (GA) was removed and weighed. Muscle weights (GA) are plotted as a percent of control, calculated as the right/left muscle weights Data are means±s.e.m., n =5-10 mice. FIGS. 30A-30D(B).

Muscle fiber size and variability were maintained in muscles from MAFbx deficient mice after denervation. Cross-sections taken from the tibialis anterior muscle were stained with an antibody against laminin (Sigma). In FIGS. 30A-30D(C), representative cross-sections are shown from the tibialis anterior: wild type (+/+), control left-side (upper left); wild type (+/+), 14-day denervated right side (lower left); homozygous (−/−), control left side (upper right); homozygous, 14-day denervated right side.

For a detailed description of the methodologies that may be employed in the creation of knockout animals, as discussed herein, see U.S. application Ser. No. 09/732,234 filed Dec. 7, 2000 which claims priority to U.S. Application Serial No. 60/244,665 filed Oct. 31, 2000, the contents of which is hereby incorporated by reference.

Through out this application, the terminology MURF1 and MURF3 are used, as is MAFbx. In our previously filed priority applications, the terminology MA-16 And MAFBXwere used. The change in terms represents a change in nomenclature and the molecules will be more accurately identified by their sequences.

### Deposit of Biological Material

The following clones were deposited with the American Type Culture Collection (ATCC®), 10801 University Boulevard, Manassas, Va. 20110-2209, on Dec. 10, 1999:

| | Patent Deposit |
|---|---|
| Clone | Designation |
| Human MA61K8 in Stratagene T3/T7 vector | PTA-1048 |
| Human MA16C8 in Stratagene T3/T7 vector | PTA-1049 |
| Human MA61D18 in Stratagene T3/T7 vector | PTA-1050 |

The present invention is not to be limited in scope by the specific embodiments described herein. Indeed, various modifications of the invention in addition to those described herein will become apparent to those skilled in the art from the foregoing description and accompanying figures.

We claim:

1. An isolated nucleic acid molecule comprising a nucleotide sequence which encodes a protein comprising the amino acid sequence as set forth in FIGS. 7, 9, and 17.

2. An isolated nucleic acid molecule which encodes MURF1, or a fragment thereof, having a sequence selected from the group consisting of
(a) the nucleotide sequence comprising the coding region of MURF1 as set forth in FIGS. 6, 8, or 17;
(b) a nucleotide sequence who complement hybridizes under stringent conditions to the nucleotide sequences of (a) and which encodes a molecule having the biological activity of MURF1; or
(c) a nucleotide sequence which, but for the degeneracy of the genetic code would hybridize to a complement of the nucleotide sequence of (a) or the complement of (b), and which encodes a molecule having the biological activity of MURF1.

3. An isolated nucleic acid molecule which is derived from a mammalian genome that:
a) hybridizes under stringent conditions to the nucleic acid molecule of FIGS. 6, 8, or 16; and
b) encodes a gene product which contains a ring domain

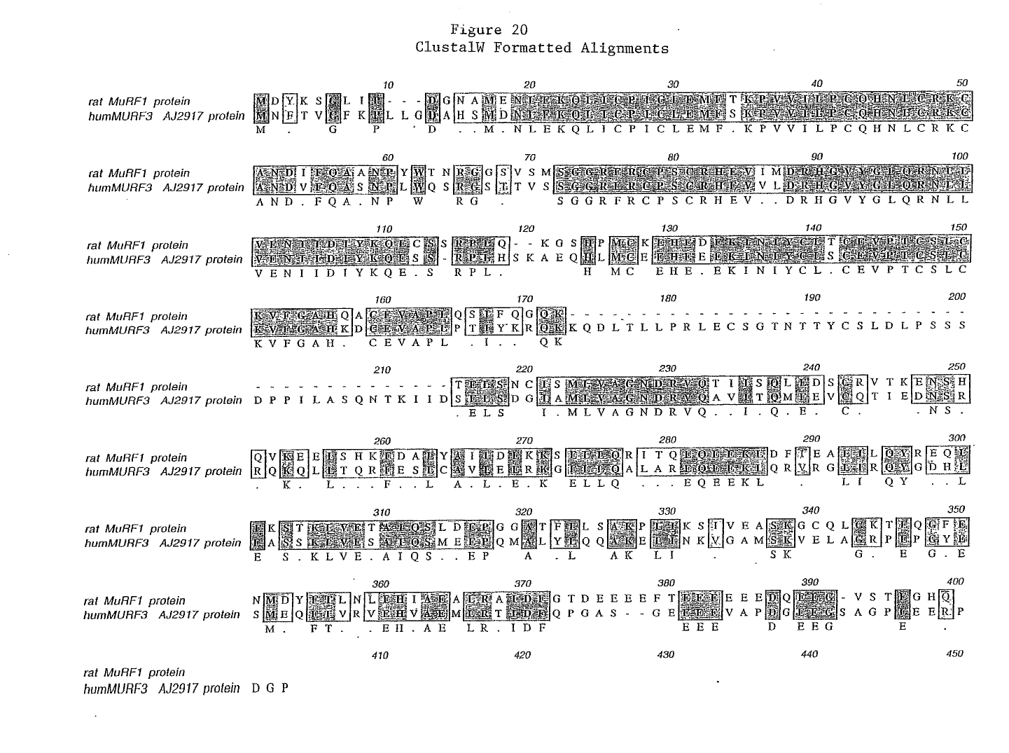

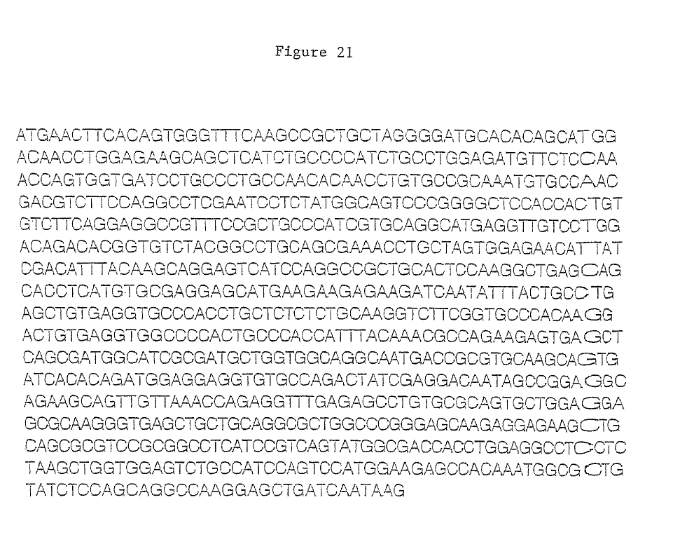

4. An isolated nucleic acid molecule which encodes MURF1, or a fragment thereof, having a sequence selected from the group consisting of
(a) the nucleotide sequence comprising the coding region of MURF1 as set forth in FIGS. 6, 8, or 16;
(b) a nucleotide sequence who complement hybridizes under stringent conditions to the nucleotide sequences of (a) and which encodes a molecule having the biological activity of MURF1; or
(c) a nucleotide sequence which, but for the degeneracy of the genetic code would hybridize to a complement of the nucleotide sequence of (a) or the complement of (b), and which encodes a molecule having the biological activity of MURF1; and
(d) does not encompass the nucleotide sequences which encodes MURF3 (FIG. 21)

5. An isolated polypeptide encoded by the nucleic acid molecule of claim 1,2,3, or 4.

6. A vector which comprises a nucleic acid molecule of claim 1, 2, 3, or 4.

7. A vector according to claim 6, wherein the nucleic acid molecule is operatively linked to an expression control sequence capable of directing its expression in a host cell.

8. A host-vector system for the production of MURF1 polypeptide which comprises a host cell transformed with the vector of claim 6.

9. A host-vector system according to claim 8 wherein the host cell is a bacterial, yeast, insect or mammalian cell.

10. A transgenic animal having cells which harbor a transgene comprising the nucleic acid of claims 1,2,3, or 4.

11. An animal inactivated in the loci comprising the nucleotide sequence of claims 1,2,3, or 4.

12. An antibody which binds the MURF1 polypeptide of claim 5.

13. A MURF1 antagonist for use in a method of inhibiting atrophy, inducing hypertrophy, decreasing ubiquitination, interfering with the ubiquitin pathway, or modulating MURF1 expression or activity.

14. An antagonist of the MURF1 pathway for use in a method of inhibiting atrophy, inducing hypertrophy, decreasing ubiquitination, interfering with the ubiquitin pathway, or modulating MURF1 expression or activity.

15. A method of screening compounds useful for the treatment of muscle atrophy or detecting atrophy and related diseases and disorders comprising contacting a muscle cell expressing MURF1 with a compound and detecting a change in the MURF1 protein activity or ubiquitination.

16. The method of claim 15 wherein the change is measured by PCR, Taqman PCR, phage display systems, gel electrophoresis, yeast-two hybrid assay, Northern or Western analysis, immunohistochemistry, a conventional scintillation camera, a gamma camera, a rectilinear scanner, a PET scanner, a SPECT scanner, a MRI scanner, a NMR scanner, or an X-ray machine.

17. The method of claim 15 where in the change in MURF1 protein activity is detected by detecting a change in the interaction of MURF1 with one or more proteins, by detecting a change in the interaction of the ring domain with another protein, or by detecting a change in the level of ubiquitination of one or more of the proteins in the ubiquitin pathway.

18. The method of claim 15 in which one of the one or more proteins is the substrate of MURF1.

19. The method of claim 15 wherein the muscle cell is of skeletal muscle origin.

20. The method of claim 15 wherein the muscle cells are cultured cells.

21. The method of claim 15 wherein the muscle cells are obtained from a transgenic organism.

22. The method of claim 21 wherein the transgenic organism includes, but is not limited to a mouse, rat, rabbit, sheep, cow or primate.

23. The method of claim 15 wherein the muscle cells are within a transgenic organism.

24. The method of claim 23 wherein the transgenic organism includes, but is not limited to a mouse, rat, rabbit, sheep, cow or primate.

25. The method of claim 15 wherein the MURF1 and the molecule capable of detecting MURF1 are nucleic acids.

26. The method of claim 15 wherein the MURF1 and the molecule capable of detecting MURF1 are polypeptides.

27. The method of claim 15 wherein the compound is a substrate for MURF1.

28. The method of claim 15 wherein the change in protein expression is demonstrated by a change in amount of protein of one or more of the proteins in the ubiquitin pathway.

29. A method of detecting muscle atrophy in an animal comprising measuring MURF1 in a patient sample.

30. A method of inhibiting atrophy or inducing hypertrophy by modulating MURF1 or a ring domain thereof.

31. A method of treating illnesses, syndromes or disorders associated with muscle atrophy comprising administering to an animal a compound that modulates the MURF1 pathway, ubiquitination, the expression or activity of MURF1 or the ring domain of MURF1, such that symptoms are alleviated.

32. The method of claim 31 such that the animal is a mammal.

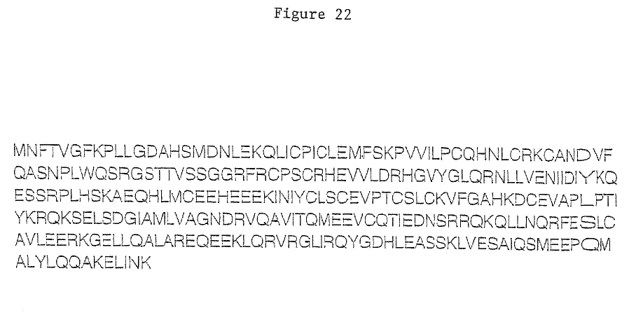

33. An isolated nucleic acid molecule comprising a nucleotide sequence which encodes a protein comprising the amino acid sequence as set forth in FIG. 22

34 An isolated nucleic acid molecule which encodes MURF3, or a fragment thereof, having a sequence selected from the group consisting of
(a) the nucleotide sequence comprising the coding region of MURF1 as set forth in FIG. 21;
(b) a nucleotide sequence who complement hybridizes under stringent conditions to the nucleotide sequences of (a) and which encodes a molecule having the biological activity of MURF3; or
(c) a nucleotide sequence which, but for the degeneracy of the genetic code would hybridize to a complement of the nucleotide sequence of (a) or the complement of (b), and which encodes a molecule having the biological activity of MURF3.

35. An isolated nucleic acid molecule which is derived from a mammalian genome that:
a) hybridizes under stringent conditions to the nucleic acid molecule of SFIG. 21; and
b) encodes a gene product which contains a ring domain

36. An isolated nucleic acid molecule which encodes MURF3, or a fragment thereof, having a sequence selected from the group consisting of
(a) the nucleotide sequence comprising the coding region of MURF1 as set forth in FIG. 21
(b) a nucleotide sequence who complement hybridizes under stringent conditions to the nucleotide sequences of (a) and which encodes a molecule having the biological activity of MURF3; or
(e) a nucleotide sequence which, but for the degeneracy of the genetic code would hybridize to a complement of the nucleotide sequence of (a) or the complement of (b), and which encodes a molecule having the biological activity of MURF3; and
(f) does not encompass the nucleotide sequences which encodes MURF1(FIGS. 6, 8, or 16).

37. An isolated polypeptide encoded by the nucleic acid molecule of claim 33, 34, 35, or 36.

38. A vector which comprises a nucleic acid molecule of claim 33,34, 35, or 36.

39. A vector according to claim 38, wherein the nucleic acid molecule is operatively linked to an expression control sequence capable of directing its expression in a host cell.

40. A host-vector system for the production of MURF3 polypeptide which comprises a host cell transformed with the vector of claim 38.

41. A host-vector system according to claim 40 wherein the host cell is a bacterial, yeast, insect or mammalian cell.

42. A transgenic animal having cells which harbor a transgene comprising the nucleic acid of claim 33,34, 35, or 36.

43. An animal inactivated in the loci comprising the nucleotide sequence of claim 33,34, 35, or 36.

44. An antibody which binds the MURF3 polypeptide of claim 37.

45. A MURF3 antagonist for use in a method of inhibiting atrophy, inducing hypertrophy, decreasing ubiquitination, interfering with the ubiquitin pathway, or modulating MURF3 expression or activity.

46. An antagonist of the MURF3 pathway for use in a method of inhibiting atrophy, inducing hypertrophy, decreasing ubiquitination, interfering with the ubiquitin pathway, or modulating MURF3 expression or activity.

47. A method of screening compounds useful for the treatment of muscle atrophy or detecting atrophy and related diseases and disorders comprising contacting a muscle cell expressing MURF3 with a compound and detecting a change in the MURF3 protein activity or ubiquitination.

48. The method of claim 47 wherein the change is measured by PCR, Taqman PCR, phage display systems, gel electrophoresis, yeast-two hybrid assay, Northern or Western analysis, immunohistochemistry, a conventional scintillation camera, a gamma camera, a rectilinear scanner, a PET scanner, a SPECT scanner, a MRI scanner, a NMR scanner, or an X-ray machine.

49. The method of claim 47 where in the change in the MURF3 protein activity is detected by detecting a change in the interaction of the MURF3 with one or more proteins, by detecting a change in the interaction of the ring domain with another protein, or by detecting a change in the level of ubiquitination of one or more of the proteins in the ubiquitin pathway.

50. The method of claim 47 in which one of the one or more proteins is the substrate of MURF3.

51. The method of claim 47 wherein the muscle cell is of skeletal muscle origin.

52. The method of claim 47 wherein the muscle cells are cultured cells.

53. The method of claim 47 wherein the muscle cells are obtained from a transgenic organism.

54. The method of claim 53 wherein the transgenic organism includes, but is not limited to a mouse, rat, rabbit, sheep, cow or primate.

55. The method of claim 47 wherein the muscle cells are within a transgenic organism.

56. The method of claim 55 wherein the transgenic organism includes, but is not limited to a mouse, rat, rabbit, sheep, cow or primate.

57. The method of claim 47 wherein the MURF3 and the molecule capable of detecting MURF3 are nucleic acids.

58. The method of claim 47 wherein the MURF3 and the molecule capable of detecting MURF3 are polypeptides.

59. The method of claim 47 wherein the compound is a substrate for MURF3.

60. The method of claim 47 wherein the change in protein expression is demonstrated by a change in amount of protein of one or more of the proteins in the ubiquitin pathway.

61. A method of detecting muscle atrophy in an animal comprising measuring MURF3 in a patient sample.

62. A method of inhibiting atrophy or inducing hypertrophy by modulating MURF3 or a ring domain thereof.

63. A method of treating illnesses, syndromes or disorders associated with muscle atrophy comprising administering to an animal a compound that modulates the MURF3 pathway, ubiquitination, the expression or activity of MURF3 such that symptoms are alleviated.

64. The method of claim 63 such that the animal is a mammal.

65. The method of claim 63 such that the mammal is a human.


SEQUENCE LISTING (48 sequences)

SEQ ID NO: 1 - DNA, 29 bp, mouse
gaacacaggaggagaaactggaacatgtc

SEQ ID NO: 2 - DNA, 24 bp, mouse
cccgaaatggcagtatttctgcag

SEQ ID NO: 3 - protein, 17 aa, rat
DEEEEFTEEEEEEDQEE

SEQ ID NO: 4 - DNA, 29 bp, mouse
gaacacaggaggagaaactggaacatgtc

SEQ ID NO: 5 - DNA, 24 bp, mouse
cccgaaatggcagtatttctgcag

SEQ ID NO: 6 - protein, 39 aa, Homo sapiens
LPLCLQLNLMQRLSDGRDLVSLGQAAPDLHVLSEDRLLW

SEQ ID NO: 7 - protein, 39 aa, Homo sapiens
LPEDVLFHILKWLSVEDILAVRAVHSQLKDLVDNHASVW

SEQ ID NO: 8 - protein, 39 aa, Homo sapiens
LPEPLLLRVLAALPAAELVQACRLVCLRWKELVDGAPLW

SEQ ID NO: 9 - protein, 42 aa, Homo sapiens
LPGEVLEYILCCGLTSAADIGRVSSTCRRLRKLCQSSGKVWK

SEQ ID NO: 10 - protein, 39 aa, Homo sapiens
LPLHMLNNILYRFSDGWDIITLGQVTPTLYMLSEDRQLW

SEQ ID NO: 11 - protein, 37 aa, Homo sapiens
LPYELAINIFQYLDRKELGRCAQVCTWKVIAEDEVLW

SEQ ID NO: 12 - protein, 38 aa, Homo sapiens
LPTDPLLLILSFLDYRDLINCCYVSRRLSQLSSHDPLW

SEQ ID NO: 13 - protein, 37 aa, Homo sapiens
LPKELALYVLSFLEPKDLLQAAQTCRWRILAEDNLLW

SEQ ID NO: 14 - protein, 36 aa, Homo sapiens
LPIDVQLYILSPLSPHDLCQLGSTNHYWNETVRDPI

SEQ ID NO: 15 - protein, 39 aa, Homo sapiens
LPENILLELFIHIPARQLLLRCRPVCSLWRDLIDLVTLW

SEQ ID NO: 16 - protein, 360 aa, mouse
  misc_feature (353)....(353): Xaa = any amino acid
MPFLGQDWRSPGWSWIKTEDGWKRCDPCSHELRSEDSQYTINHSIILNSGEEEIFNNECEYAAKKRKKEHFGNDTAAHSFYREKWIYVHKESTKERHGYCTLGEAFNRLDFSSALQDIRRFTYVVKLLQLIAKSQLTSLSGVAQKNYFNILDKIVQKVLDDHQNPRLLKGLLQDLSSTLGILVRGVGKSVLVGNINIWTCRLETVLSWQQQLQNLQVTKQVNTGLTLSDLPLHMLNNILYRFSDGWDLVTLGQVTPTLYMLSEDRRLWKRLCQYHFAEQQFCRHLILSEKGHLEWKLMYFTLQKYYPTKEQYGDTLHFCRHCSILFWKDSGHPCTRADPDSCFTPVSPEHEIXLFKFXWC

SEQ ID NO: 17 - protein, 272 aa, Homo sapiens
LILTSVLLFQRHGYCTLGEAFNRLDFSSAIQDIRTFNYVVKLLQLIAKSQLTSLSGVAQKNYFNILDKIVQKVLDDHHNPRLLKDLLQDLSSTLCILTRGVGKSVLVGNINIWTCRLETILAWQQQLQDLQMTKQVNNGLTLSDLPLHMLNNILYRFSDGWDILTLGQVTPTLYMLSEDRQLWKKLCQYHFAEKQFCRHLLLSEKGHLEWKLMYFALQKHYPAKEQYGDTLHFCRHCSILFWKDSGHPCTAADPDSCFTPVSPQHEIDLFKF

SEQ ID NO: 18 - protein, 344 aa, Homo sapiens
MPFLGQDWRSPGQNWYKTADGWKRFLDEKSGSFVSDLSSYCNKEVYNKENLFNSLNYDVAAKKRKKDMLNSKTKTQYLHQEKWTYVHKGSTKERHGYCTLGEAFNRLDESTAILDSRRENYVVRLLELTAKSQLTSLSGIAQKNFMNLLEKVVLKVLEDQQNIRLIRELLQTLYTSLCTLVQRVGKSVLVGNINMWVYRMETILHWQQQLNNIQITRPAFKGLTFTDLPLCLQLNIMQRLSDGRDLVSLGQAAPDLHVLSEDRLLWKKLCQYHFSERQIRKRLILSDKGQLDWKKMYFKLVRCYPRREQYGVTLQLCKHCHILSWKGTDHPCTANNPESCSVSL

SEQ ID NO: 19 - protein, 350 aa, rat
MPFLGQDWRSPGQSWVKTADGWKRFLDEKSGTFVSDLSSYCNKENLFNSLNYDVAAKKRKKDIQNSKTKTQYFHQEKWIYVHKGSTKERHGYCTLGEAFNRLDFSTAILDSRRENYVVRLLELTAKSQLTSLSGIAQKNFMNLLEKVVLKVLEDQQNIRLIRELLQTLYTSLCTLVQRVGKSVLVGNLNMWVYRMETTLHWQQQLNSIQISRPAFKGLTITDLPVCLQLNIMQRLSDGRDLVSLGQAAPDLHVLSEDRLLWKRLCQYHFSERQIRKRLLLSDKGQLDWKKMYFKLVRCYPRREQYGVTLQLCKHCHILSWKGTDHPCTANNPESCSVSLSPQDEINLFKF

SEQ ID NO: 20 - DNA, 1053 bp, rat
atggattataaatctggcttgattccggacggaaatgctatggagaacctggagaagcagctcatctgccccatctgccttgagatgtttaccaagcctgtggtcatcctgccctgccagcacaacctctgccggaagtgtgccaacgacatcttccaggctgccaatccctactggaccaaccgcggtggctcggtgtccatgtctggaggtcgtttccgctgcccctcgtgccgccatgaagtgatcatggaccggcatggggtgtacggtctgcagaggaacctgctggtggagaacatcatcgacatctacaagcaggaatgctccagtcggcccctgcagaaaggcagccacccgatgtgcaaggaacacgaagacgagaaaatcaacatctactgtctcacgtgcgaggtgcctacttgctccttgtgcaaggtgttcggggctcaccaggcctgtgaagttgcccccttacaaagcatcttccaaggacagaagactgaactgagcaattgcatctccatgctggtggcagggaacgaccgagttcagactatcatctcgcagctggaggactcctgccgagtgaccaaggaaaacagccaccaggtgaaggaggaactgagccacaagtttgacgccctctacgccatcctggacgagaagaagagtgagctgctgcagcggatcactcaggagcaggaggagaagctggacttcatcgaggccctgatcctccagtaccgagagcagttggaaaagtcgaccaagcttgtggaaacagccatccagtccctggatgagcccggaggggccaccttcctcttgagtgccaagccgctcatcaagagcattgtagaagcttccaagggctgccagctggggaagacagaacaaggctttgagaacatggactactttactctgaatttagaacacatagcagaggccttgagggccatcgactttgggacagatgaggaggaggagtttactgaagaggaggaggaggaggatcaagaagagggcgtgtccacagagggacaccaa

SEQ ID NO: 21 - protein, 351 aa, rat
MDYKSGLIPDGNAMENLEKQLICPICLEMFTKPVVILPCQHNLCRKCANDIFQAANPYWTNRGGSVSMSGGRFRCPSCRHEVIMDRHGVYGLQRNLLVENIIDIYKQECSSRPLQKGSHPMCKEHEDEKINIYCLTCEVPTCSLCKVFGAHQACEVAPLQSIFQGQKTELSNCISMLVAGNDRVQTIISQLEDSCRVTKENSHQVKEELSHKFDALYAILDEKKSELLQRITQEQEEKLDFIEALILQYREQLEKSTKLVETAIQSLDEPGGATFLLSAKPLIKSIVEASKGCQLGKTEQGFENMDYFTLNLEHIAEALRAIDFGTDEEEEFTEEEEEEDQEEGVSTEGHQ

SEQ ID NO: 22 - DNA, 2097 bp, Homo sapiens
ttctcaggtactttatcggacctctcacatggctgcatgcccagaaatgtgatgatattgttgacagcctcttcaagggttttggtagaactgagggcaaaggtttcctctttctcaaaggtatctcccacctcttcccaagcagcagcaaagttaggctgacctcgtctgttatgtaaaggatgcgtagggatgggagggcgatgaggactaggatgatggcgggcaggatagttcagacggtttccatttcctgagcgtctgagatgttagtattagttagttttgttgtgagtgttagaattcgggcaccaggagaaggaagccaacaggatccgacccggtgttttgtgacaaaggcaagacccccaggtctacttagagcaaagttagtagaggaggcagctaggcgtggctctcattccttcccacagaatggattataagtcgagcctgatccaggatgggaatcccatggagaacttggagaagcagctgatctgccctatctgcctggagatgtttaccaagccagtggtcatcttgccgtgccagcacaacctgtgccggaagtgtgccaatgacatcttccaggctgcaaatccctactggaccagccggggcagctcagtgtccatgtctggaggccgtttccgctgccccacctgccgccacgaggtgatcatggatcgtcacggagtgtacggcctgcagaggaacctgctggtggagaacatcatcgacatctacaaacaggagtgctccagtcggccgctgcagaagggcagtcaccccatgtgcaaggagcacgaagatgagaaaatcaacatctactgtctcacgtgtgaggtgcccacctgctccatgtgcaaggtgtttgggatccacaaggcctgcgaggtggccccattgcagagtgtcttccagggacaaaagactgaactgaataactgtatctccatgctggtggcggggaatgaccgtgtgcagaccatcatcactcagctggaggattcccgtcgagtgaccaaggagaacagtcaccaggtaaaggaagagctgagccagaagtttgacacgttgtatgccatcctggatgagaagaaaagtgagttgctgcagcggatcacgcaggagcaggaggaaaagcttagcttcatcgaggccctcatccagcagtaccaggagcagctggacaagtccacaaagctggtggaaactgccatccagtccctggacgagcctgggggagccaccttcctcttgactgccaagcaactcatcaaaagcattgtggaagcttccaagggctgccagctggggaagacagagcagggctttgagaacatggacttctttactttggatttagagcacatagcagacgccctgagagccattgactttgggacagatgaggaagaggaagaattcattgaagaagaagatcaggaagaggaagagtccacagaagggaaggaagaaggacaccagtaaggagctggatgaatgagaggcccccagatgcagagagactggagagggtggggaggggcccagcggccttggtgacaggcccagggtgggaggggtcggggcccctggaggggcaatggggaggtgatgtcttctctctgctcagagagcagggactagggtaggaccctcaccgctgcgtccagcagacactgaaccagaattggaaacgtgcttgaaacaatcacacaggacacttttctacattggtgcaaaatggaatattttgtacatttttaaaatgtgatttttgtatatacttgtatatgtatgccaatttggtgctttttgtaaaggaacttttgtataataatgcctggtcgttgggtgacctgcgattgtcagaaagaggggaaggaagccaggttgatacagctgcccacttcctttcctgagcaggaggatggggtagcactcacagggacgatgtgctgtatttcagtgcctatcccagacatacggggtggtaactgagtttgtgttatatgttgttttaataaatgcacaatgctctcttcctgttcttcaaaaaaaaaaaaaaaaaaaaaaaaaaaaaaaaaa

SEQ ID NO: 23 - protein, 340 aa, Homo sapiens
MENLEKQLICPICLEMFTKPVVILPCQHNLCRKCANDIFQAANPYWTSRGSSVSMSGGRFRCPTCRHEVIMDRHGVYGLQRNLLVENIIDIYKQECSSRPLQKGSHPMCKEHEDEKINIYCLTCEVPTCSMCKVFGIHKACEVAPLQSVFQGQKTELNNCISMLVAGNDRVQTIITQLEDSRRVTKENSHQVKEELSQKFDTLYAILDEKKSELLQRITQEQEEKLSFIEALIQQYQEQLDKSTKLVETAIQSLDEPGGATFLLTAKQLIKSIVEASKGCQLGKTEQGFENMDFFTLDLEHIADALRAIDFGTDEEEEEFIEEEDQEEEESTEGKEEGHQ

SEQ ID NO: 24 - DNA, 1050 bp, rat
atgccgttccttggtcaggactggcggtccccgggccagagctgggtgaagacggcggacggctggaagcgcttcttggatgagaaaagcggcaccttcgtgagcgacctcagcagttactgcaacaaggagaatctgttcaacagcctgaactacgatgttgcagccaagaagagaaagaaagacatacagaacagcaaaaccaaaactcagtatttccatcaggagaagtggatctatgttcacaaagggagtactaaggagcgccatggatactgcactttgggggaagctttcaacagactggacttctcgactgccatcctggattccagaagattcaactacgtagtaaggctgttggagctgatagcaaagtcacagctcacatccctgagtggcatcgcccaaaagaacttcatgaacattttggaaaaagtagtactgaaagttcttgaagaccagcaaaacataagactcatacgggaacttctccagaccctctacacatccttatgcacgctggtccagagagtcggcaagtccgtgctggtgggcaacatcaacatgtgggtgtatcgaatggagaccactctacactggcaacagcagctgaacagcatccagatcagcaggccggccttcaaaggtctcacgatcaccgacctgcctgtgtgcttacaactgaacatcatgcagaggctgagcgatgggcgggacctggtcagcctgggccaggcagccccagacctgcatgtgctcagtgaagaccggctactgtggaagagactctgccagtaccacttctcagagcggcagatccgcaagcgattgatcttgtctgacaaagggcagctggattggaagaagatgtactttaagcttgtgcgatgttacccaagaagagaacagtatggggtcaccctgcagctttgcaaacactgccacattctctcctggaagggcactgaccatccatgcacggccaacaacccagagagctgctccgtctcactttcaccccaagactttattaacttgttcaagttc

SEQ ID NO: 25 - protein, 350 aa, rat
MPFLGQDWRSPGQSWVKTADGWKRFLDEKSGTFVSDLSSYCNKENLFNSLNYDVAAKKRKKDIQNSKTKTQYFHQEKWIYVHKGSTKERHGYCTLGEAFNRLDFSTAILDSRRFNYVVRLLELIAKSQLTSLSGIAQKNFMNILEKVVLKVLEDQQNIRLIRELLQTLYTSLCTLVQRVGKSVLVGNINMWVYRMETTLHWQQQLNSIQISRPAFKGLTITDLPVCLQLNIMQRLSDGRDLVSLGQAAPDLHVLSEDRLLWKRLCQYHFSERQIRKRLILSDKGQLDWKKMYFKLVRCYPRREQYGVTLQLCKHCHILSWKGTDHPCTANNPESCSVSLSPQDFINLFKF

SEQ ID NO: 26 - DNA, 1035 bp, Homo sapiens
atgccattcctcgggcaggactggcggtcccccgggcagaactgggtgaagacggccgacggctggaagcgcttcctggatgagaagagcggcagtttcgtgagcgacctcagcagttactgcaacaaggaggtatacaataaggagaatcttttcaacagcctgaactatgatgttgcagccaagaagagaaagaaggacatgctgaatagcaaaaccaaaactcagtatttccaccaagaaaaatggatctatgttcacaaaggaagtactaaagagcgccatggatattgcaccctgggggaagctttcaacagactggacttctcaactgccattctggattccagaagatttaactacgtggtccggctgttggagctgatagcaaagtcacagctcacatccctgagtggcatcgcccaaaagaacttcatgaatattttggaaaaagtggtactgaaagtccttgaagaccagcaaaacattagactaataagggaactactccagaccctctacacatccttatgtacactggtccaaagagtcggcaagtctgtgctggtcgggaacattaacatgtgggtgtatcggatggagacgattctccactggcagcagcagctgaacaacattcagatcaccaggcctgccttcaaaggcctcaccttcactgacctgcctttgtgcctacaactgaacatcatgcagaggctgagcgacgggcgggacctggtcagcctgggccaggctgcccccgacctgcacgtgctcagcgaagaccggctgctgtggaagaaactctgccagtaccacttctccgagcggcagatccgcaaacgattaattctgtcagacaaagggcagctggattggaagaagatgtatttcaaacttgtccgatgttacccaaggaaagagcagtatggagatacccttcagctctgcaaacactgtcacatcctttcctggaagggcactgaccatccgtgcactgccaataacccagagagctgctccgtttcactttga

SEQ ID NO: 27 - protein, 344 aa, Homo sapiens
MPFLGQDWRSPGQNWVKTADGWKRFLDEKSGSFVSDLSSYCNKEVYNKENLFNSLNYDVAAKKRKKDMLNSKTKTQYFHQEKWIYVHKGSTKERHGYCTLGEAFNRLDFSTAILDSRRFNYVVRLLELIAKSQLTSLSGIAQKNFMNILEKVVLKVLEDQQNIRLIRELLQTLYTSLCTLVQRVGKSVLVGNINMWVYRMETILHWQQQLNNIQITRPAFKGLTFTDLPLCLQLNIMQRLSDGRDLVSLGQAAPDLHVLSEDRLLWKKLCQYHFSERQIRKRLILSDKGQLDWKKMYFKLVRCYPRKEQYGDTLQLCKHCHILSWKGTDHPCTANNPESCSVSL

SEQ ID NO: 28 - protein, 64 aa, Homo sapiens
CPICLEMFSKPVVILPCQHNLCRKCANDVFQASNPLWQSRGSTTVSSGGRFRCPSCRHEVVIDR

SEQ ID NO: 29 - protein, 57 aa, Homo sapiens
CPICLELLEDPLLLPCAHSLCFSCAHRILVSSCSSGESIEPITAFQCPTCRYVISLN

SEQ ID NO: 30 - protein, 57 aa, Homo sapiens
CPICCSLFDDPRVLPCSHNFCKKCLEGILEGSVRNSLWRPAPFKCPTCRKETSATGI

SEQ ID NO: 31 - protein, 57 aa, Homo sapiens
CPICLELFEDPLLLPCAHSLCFSCAHRILVSSCSSGESLEPITAFQCPTCRYVISLN

SEQ ID NO: 32 - DNA, 630 bp, rat
atggactacaaagacgatgacgacaaagattataaatctggcttgattccggacggaaatgctatggagaacctggagaagcagctcatctgccccatctgccttgagatgtttaccaagcctgtggtcatcctgccctgccagcacaacctctgccggaagtgtgccaacgacatcttccaggctgccaatccctactggaccaaccgcggtggctcggtgtccatgtctggaggtcgtttccgctgcccctcgtgccgccatgaagtgatcatggaccggcatggggtgtacggtctgcagaggaacctgctggtggagaacatcatcgacatctacaagcaggaatgctccagtcggcccctgcagaaaggcagccacccgatgtgcaaggaacacgaagacgagaaaatcaacatctactgtctcacgtgcgaggtgcctacttgctccttgtgcaaggtgttcggggctcaccaggcctgtgaagttgcccccttacaaagcatcttccaaggacagaagactgaactgagcaattgcatctccatgctggtggcagggaacgaccgagttcagactatcatctcgcagctggaggactcctgccgagtgaccaaggtgagggtg

SEQ ID NO: 33 - protein, 202 aa, rat
MDYKSGLIPDGNAMENLEKQLICPICLEMFTKPVVILPCQHNLCRKCANDIFQAANPYWTNRGGSVSMSGGRFRCPSCRHEVIMDRHGVYGLQRNLLVENIIDIYKQECSSRPLQKGSHPMCKEHEDEKINIYCLTCEVPTCSLCKVFGAHQACEVAPLQSIFQGQKTELSNCISMLVAGNDRVQTIISQLEDSCRVTKVRV

SEQ ID NO: 34 - DNA, 1065 bp, Homo sapiens
atgccattcctcgggcaggactggcggtcccccgggcagaactgggtgaagacggccgacggctggaagcgcttcctggatgagaagagcggcagtttcgtgagcgacctcagcagttactgcaacaaggaggtatacaataaggagaatcttttcaacagcctgaactatgatgttgcagccaagaagagaaagaaggacatgctgaatagcaaaaccaaaactcagtatttccaccaagaaaaatggatctatgttcacaaaggaagtactaaagagcgccatggatattgcaccctgggggaagctttcaacagactggacttctcaactgccattctggattccagaagatttaactacgtggtccggctgttggagctgatagcaaagtcacagctcacatccctgagtggcatcgcccaaaagaacttcatgaatattttggaaaaagtggtactgaaagtccttgaagaccagcaaaacattagactaataagggaactactccagaccctctacacatccttatgtacactggtccaaagagtcggcaagtctgtgctggtcgggaacattaacatgtgggtgtatcggatggagacgattctccactggcagcagcagctgaacaacattcagatcaccaggcctgccttcaaaggcctcaccttcactgacctgcctttgtgcctacaactgaacatcatgcagaggctgagcgacgggcgggacctggtcagcctgggccaggctgcccccgacctgcacgtgctcagcgaagaccggctgctgtggaagaaactctgccagtaccacttctccgagcggcagatccgcaaacgattaattctgtcagacaaagggcagctggattggaagaagatgtatttcaaacttgtccgatgttacccaaggaaagagcagtatggagatacccttcagctctgcaaacactgtcacatcctttcctggaagggcactgaccatccgtgcactgccaataacccagagagctgctccgtttcactttcaccccaggactttatcaacttgttcaagttc

SEQ ID NO: 35 - protein, 355 aa, Homo sapiens
MPFLGQDWRSPGQNWVKTADGWKRFLDEKSGSFVSDLSSYCNKEVYNKENLFNSLNYDVAAKKRKKDMLNSKTKTQYFHQEKWIYVHKGSTKERHGYCTLGEAFNRLDFSTAILDSRRFNYVVRLLELIAKSQLTSLSGIAQKNFMNILEKVVLKVLEDQQNIRLIRELLQTLYTSLCTLVQRVGKSVLVGNINMWVYRMETILHWQQQLNNIQITRPAFKGLTFTDLPLCLQLNIMQRLSDGRDLVSLGQAAPDLHVLSEDRLLWKKLCQYHFSERQIRKRLILSDKGQLDWKKMYFKLVRCYPRKEQYGDTLQLCKHCHILSWKGTDHPCTANNPESCSVSLSPQDFINLFKF

SEQ ID NO: 36 - protein, 351 aa, rat; identical to SEQ ID NO: 21

SEQ ID NO: 37 - protein, 396 aa, Homo sapiens
MNFTVGFKPLLGDAHSMDNLEKQLICPICLEMFSKPVVLLPCQHNLCRKCANDVFQASNPLWQSRGSTTVSSGGRFRCPSCRHEVVLDRHGVYGLQRNLLVENIIDIYKQESSRPLHSKAEQHLMCEEHEEEKINIYCLSCEVPTCSLCKVFGAHKDCEVAPLPTIYKRQKKQDLTLLPRLECSGTNTTYCSLDLPSSSDPPILASQNTKIIDSELSDGIAMLVAGNDRVQAVITQMEEVCQTIEDNSRRQKQLLTQRFESLCAVLEERKGELLQALAREQEEKLQRVRGLIRQYGDHLEASSKLVESAIQSMEEPQMALYLQQAKELINKVGAMSKVELAGRPEPGYESMEQFTVRVEHVAEMLRTIDFQPGASGEEEVAPDGEEGSAGPEEERP

SEQ ID NO: 38 - DNA, 867 bp, Homo sapiens
atgaacttcacagtgggtttcaagccgctgctaggggatgcacacagcatggacaacctggagaagcagctcatctgccccatctgcctggagatgttctccaaaccagtggtgatcctgccctgccaacacaacctgtgccgcaaatgtgccaacgacgtcttccaggcctcgaatcctctatggcagtcccggggctccaccactgtgtcttcaggaggccgtttccgctgcccatcgtgcaggcatgaggttgtcctggacagacacggtgtctacggcctgcagcgaaacctgctagtggagaacattatcgacatttacaagcaggagtcatccaggccgctgcactccaaggctgagcagcacctcatgtgcgaggagcatgaagaagagaagatcaatatttactgcctgagctgtgaggtgcccacctgctctctctgcaaggtcttcggtgcccacaaggactgtgaggtggccccactgcccaccatttacaaacgccagaagagtgagctcagcgatggcatcgcgatgctggtggcaggcaatgaccgcgtgcaagcagtgatcacacagatggaggaggtgtgccagactatcgaggacaatagccggaggcagaagcagttgttaaaccagaggtttgagagcctgtgcgcagtgctggaggagcgcaagggtgagctgctgcaggcgctggcccgggagcaagaggagaagctgcagcgcgtccgcggcctcatccgtcagtatggcgaccacctggaggcctcctctaagctggtggagtctgccatccagtccatggaagagccacaaatggcgctgtatctccagcaggccaaggagctgatcaataag

SEQ ID NO: 39 - protein, 289 aa, Homo sapiens
MNFTVGFKPLLGDAHSMDNLEKQLICPICLEMFSKPVVILPCQHNLCRKCANDVFQASNPLWQSRGSTTVSSGGRFRCPSCRHEVVLDRHGVYGLQRNLLVENIIDIYKQESSRPLHSKAEQHLMCEEHEEEKINIYCLSCEVPTCSLCKVFGAHKDCEVAPLPTIYKRQKSELSDGIAMLVAGNDRVQAVITQMEEVCQTIEDNSRRQKQLLNQRFESLCAVLEERKGELLQALAREQEEKLQRVRGLIRQYGDHLEASSKLVESAIQSMEEPQMALYLQQAKELINK

SEQ ID NO: 40 - protein, 350 aa, rat
MPFLGQDWRSPGQSWVKTADGWKRFLDEKSGTFVSDLSSYCNKENLFNSLNYDYAAKKRKKDIQNSKTKTQYFHQEKWIYVKKGSTKERHGYCTLGEAFNRLDFSTAILDSRRTNYVVRLLELIAKSQLTSLSGIAQKNFMNILEKVVLKVLEDQQNIRLTRELLQTLYTSLCTLVQRVGKSVLVGNTNMWVYRMETTLHWQQQLNSIQISRPAFKGLTITDLPVCLQLNIMQRLSDGRDLVSLGQAAPDLHVLSEDRLLWKRLCQYHFSERQIRKRLILSDKGQLDWKKMYFKLVRCYPRREQYGVTLQLCKHCHILSWKGTDHPCTANNPESCSVSLSPQDFINLFKF

SEQ ID NO: 41 - protein, 355 aa, Homo sapiens
MPFLGQDWRSPGQNWVKTADGWKRFLDEKSGSFVSDLSSYCNKEVYNKENLFNSLNYDYAAKKRKKDMLNSKTKTQYFHQEKWIYVHKGSTKERHGYCTLGEAFNRLDFSTAILDSRRTNYVVRLLELIAKSQLTSLSGIAQKNFMNILEKVVLKVLEDQQNIRLTRELLQTLYTSLCTLVQRVGKSVLVGNTNMWVYRMETTLHWQQQLNNIQITRPAFKGLTFTDLPLCLQLNIMQRLSDGRDLVSLGQAAPDLHVLSEDRLLWKKLCQYHFSERQIRKRLILSDKGQLDWKKMYFKLVRCYPRKEQYGDTLQLCKHCHILSWKGTDHPCTANNPESCSVSLSPQDFINLFKF

SEQ ID NO: 42 - protein, 271 aa, Homo sapiens
LILTSVLLFQRHGYCTLGEAFNRLDFSSAIQDIRTENYVVKLLQLIAKSQLTSLSGVAQKNYFNILDKIVQKVLDDHHNPRLTKDLLQDLSSTLCILIRGVGKSVLVGNINIWICRLETILAWQQQLQDLQMTKQVNNGLTLSDLPLHMLNILYRFSDGWDIITLGQVTPTLYMLSEDRQLWKRLCQYHFAEKQFCRHLILSEKGHIEWKLMYFALQKHYPAKEQYGDTLHFCRHCSTLFWKDSGHPCTAADPDSCFTPVSPQHFIDLFKF

SEQ ID NO: 43 - protein, 350 aa, rat; identical to SEQ ID NO: 25

SEQ ID NO: 44 - protein, 350 aa, rat
MPFLGQDWRSPGQSWVKTADGWKRFLDEKSGLLVSDLSSYCNKENLFNSLNYDVAAKKRKKDIQNSKTKTQYFHQEKWIYVHKGSTKERHGYCTLGEALNRLDFSTAILDSRRFNYVVRLLELIAKSQLTSLSGIAQKNFMNILEKVVLKVLEDQQNIRLIRELLQTLYTSLCTLVQRVGKSVLVGNINMWVYRMETTLHWQQQLNSIQISRPAFKGLTITDLPVCLQLNIMQRLSDGRDLVSLGQAAPDLHVLSEDRLLWKRLCQYHFSERQIRKRLILSDKGQLDWKKMYFKLVRCYPRREQYGVTLQLCKHCHILSWKGTDHPCTANNPESCSVSLSPQDFINLFKF

SEQ ID NO: 45 - protein, 351 aa, rat; identical to SEQ ID NO: 21

SEQ ID NO: 46 - protein, 351 aa, rat; identical to SEQ ID NO: 21

SEQ ID NO: 47 - protein, 351 aa, rat; identical to SEQ ID NO: 21

SEQ ID NO: 48 - protein, 353 aa, Homo sapiens
MDYKSSLIQDGNPMENLEKQLICPICLEMFTKPVVILPCQHNLCRKCANDIFQAANPYWTSRGSSVSMSGGRFRCPTCRHEVIMDRHGVYGLQRNLLVENIIDIYKQECSSRPLQKGSHPMCKEHEDEKINIYCLTCEVPTCSMCKVFGIHKACEVAPLQSVFQGQKTELNNCISMLVAGNDRVQTIYTQLEDSRRVTKENSHQVKEELSQKFDTLYAILDEKKSELLQRITQEQEEKLSFIEALIQQYQEQLDKSTKLVETAIQSLDEPGGATFLLTAKQLIKSIVEASKGCQLGKTEQGFENMDFFTLDLEHIADALRAIDFGTDEEEEEFIEEEDQEEEESTEGKEEGHQ